SEQUENCE LISTING (82 sequences)

SEQ ID NO: 1 - DNA, 21 bp, Artificial Sequence
  Synthetic oligonucleotide
gatgagcaacttggacagcaa

SEQ ID NO: 2 - DNA, 21 bp, Artificial Sequence
  Synthetic oligonucleotide
ctgggctgcttatctgggaag

SEQ ID NO: 3 - DNA, 21 bp, Artificial Sequence
  Synthetic oligonucleotide
agtccactgagtaccggagac

SEQ ID NO: 4 - DNA, 21 bp, Artificial Sequence
  Synthetic oligonucleotide
catttcacgcatctggcgttc

SEQ ID NO: 5 - DNA, 21 bp, Artificial Sequence
  Synthetic oligonucleotide
ggagacggctattttgggacg

SEQ ID NO: 6 - DNA, 21 bp, Artificial Sequence
  Synthetic oligonucleotide
tccttgagtggagcttccatt

SEQ ID NO: 7 - DNA, 20 bp, Artificial Sequence
  Synthetic oligonucleotide
tcaatcatgtccagcgagtc

SEQ ID NO: 8 - DNA, 18 bp, Artificial Sequence
  Synthetic oligonucleotide
aacgaggacgagcgtttg

SEQ ID NO: 9 - DNA, 19 bp, Artificial Sequence
  Synthetic oligonucleotide
ctcctactcgtacccgcag

SEQ ID NO: 10 - DNA, 21 bp, Artificial Sequence
  Synthetic oligonucleotide
ttgaagtcgggcatcttaggc

SEQ ID NO: 11 - DNA, 21 bp, Artificial Sequence
  Synthetic oligonucleotide
ttcgcctctacagtcctgttc

SEQ ID NO: 12 - DNA, 20 bp, Artificial Sequence
  Synthetic oligonucleotide
gctcctccgggtacttatcg

SEQ ID NO: 13 - DNA, 19 bp, Artificial Sequence
  Synthetic oligonucleotide
cgtgagcacggtaaacccc

SEQ ID NO: 14 - DNA, 19 bp, Artificial Sequence
  Synthetic oligonucleotide
cgagcggatcttggtgttg

SEQ ID NO: 15 - DNA, 21 bp, Artificial Sequence
  Synthetic oligonucleotide
acagacctcaatcgctcagga

SEQ ID NO: 16 - DNA, 22 bp, Artificial Sequence
  Synthetic oligonucleotide
aggggtaaatctggatactggc

SEQ ID NO: 17 - DNA, 21 bp, Artificial Sequence
  Synthetic oligonucleotide
cgttccgggcttatacaatgt

SEQ ID NO: 18 - DNA, 19 bp, Artificial Sequence
  Synthetic oligonucleotide
ctcgtccgtcttgtcgcag

SEQ ID NO: 19 - DNA, 23 bp, Artificial Sequence
  Synthetic oligonucleotide
ttcccagaacaaacttcttgtgc

SEQ ID NO: 20 - DNA, 21 bp, Artificial Sequence
  Synthetic oligonucleotide
gcatgttgaaggaactcggct

SEQ ID NO: 21 - DNA, 19 bp, Artificial Sequence
  Synthetic oligonucleotide
accggcctattacgactgc

SEQ ID NO: 22 - DNA, 20 bp, Artificial Sequence
  Synthetic oligonucleotide
tgctggtagcctgagttgga

SEQ ID NO: 23 - DNA, 21 bp, Artificial Sequence
  Synthetic oligonucleotide
ggaagacaaacctacagtcgc

SEQ ID NO: 24 - DNA, 23 bp, Artificial Sequence
  Synthetic oligonucleotide
tcctggtcagataggggttaaaa

SEQ ID NO: 25 - DNA, 20 bp, Artificial Sequence
  Synthetic oligonucleotide
tacgtggactcgttcctgct

SEQ ID NO: 26 - DNA, 19 bp, Artificial Sequence
  Synthetic oligonucleotide
cgtcgccttggactggaag

SEQ ID NO: 27 - DNA, 21 bp, Artificial Sequence
  Synthetic oligonucleotide
actcgctcatctctcacgaca

SEQ ID NO: 28 - DNA, 21 bp, Artificial Sequence
  Synthetic oligonucleotide
gacggaaaatcgctacagtcc

SEQ ID NO: 29 - DNA, 21 bp, Artificial Sequence
  Synthetic oligonucleotide
ggactcgcttataggccatga

SEQ ID NO: 30 - DNA, 20 bp, Artificial Sequence
  Synthetic oligonucleotide
gcaaaactacacgaggcgaa

SEQ ID NO: 31 - DNA, 21 bp, Artificial Sequence
  Synthetic oligonucleotide
acatgccctcgcaatgtaact

SEQ ID NO: 32 - DNA, 21 bp, Artificial Sequence
  Synthetic oligonucleotide
gagaggtaggacggataggtg

SEQ ID NO: 33 - DNA, 22 bp, Artificial Sequence
  Synthetic oligonucleotide
atgtttaactcggtcaacctgg

SEQ ID NO: 34 - DNA, 22 bp, Artificial Sequence
  Synthetic oligonucleotide
gcatgtagtaagtgcaactggg

SEQ ID NO: 35 - DNA, 20 bp, Artificial Sequence
  Synthetic oligonucleotide
tctccgagtcctcgtgggga

SEQ ID NO: 36 - DNA, 21 bp, Artificial Sequence
  Synthetic oligonucleotide
gcaaaacaccagcgccttcta

SEQ ID NO: 37 - DNA, 23 bp, Artificial Sequence
  Synthetic oligonucleotide
tccttggtcaggcagtataatcc

SEQ ID NO: 38 - DNA, 24 bp, Artificial Sequence
  Synthetic oligonucleotide
gtcaagggcatatcctacaacaaa

SEQ ID NO: 39 - DNA, 20 bp, Artificial Sequence
  Synthetic oligonucleotide
cggcactcagctacgctaac

SEQ ID NO: 40 - DNA, 21 bp, Artificial Sequence
  Synthetic oligonucleotide
caagtccgggttggagtagac

SEQ ID NO: 41 - DNA, 19 bp, Artificial Sequence
  Synthetic oligonucleotide
gctctctgaccccagtagc

SEQ ID NO: 42 - DNA, 19 bp, Artificial Sequence
  Synthetic oligonucleotide
gcgttgtgtggagcaagtc

SEQ ID NO: 43 - DNA, 20 bp, Artificial Sequence
  Synthetic oligonucleotide
gccccagggttatgagacta

SEQ ID NO: 44 - DNA, 19 bp, Artificial Sequence
  Synthetic oligonucleotide
tctgtccagcttggaggac

SEQ ID NO: 45 - DNA, 23 bp, Artificial Sequence
  Synthetic oligonucleotide
acactaccgtttgatgagtctgt

SEQ ID NO: 46 - DNA, 21 bp, Artificial Sequence
  Synthetic oligonucleotide
cttctggtcctcgcattctct

SEQ ID NO: 47 - DNA, 22 bp, Artificial Sequence
  Synthetic oligonucleotide
tctcaagtcgaagagacccaac

SEQ ID NO: 48 - DNA, 19 bp, Artificial Sequence
  Synthetic oligonucleotide
tgcaggtgagactcagcaa

SEQ ID NO: 49 - DNA, 21 bp, Artificial Sequence
  Synthetic oligonucleotide
acctactcttgtgtgggtgtt

SEQ ID NO: 50 - DNA, 21 bp, Artificial Sequence
  Synthetic oligonucleotide
gacatagcttggagtggttcg

SEQ ID NO: 51 - DNA, 20 bp, Artificial Sequence
  Synthetic oligonucleotide
catcacgccgtcctatgtcg

SEQ ID NO: 52 - DNA, 20 bp, Artificial Sequence
  Synthetic oligonucleotide
cgtcaaagaccgtgttctcg

SEQ ID NO: 53 - DNA, 22 bp, Artificial Sequence
  Synthetic oligonucleotide
atggcatcctactacgagatcc

SEQ ID NO: 54 - DNA, 19 bp, Artificial Sequence
  Synthetic oligonucleotide
gagagccttgcgccgatac

SEQ ID NO: 55 - DNA, 21 bp, Artificial Sequence
  Synthetic oligonucleotide
gggagtcattgtaccacgtcc

SEQ ID NO: 56 - DNA, 19 bp, Artificial Sequence
  Synthetic oligonucleotide
cgtgcgcgttgttgatctc

SEQ ID NO: 57 - DNA, 20 bp, Artificial Sequence
  Synthetic oligonucleotide
acggtcaagaccaaggatgg

SEQ ID NO: 58 - DNA, 19 bp, Artificial Sequence
  Synthetic oligonucleotide
agcgtgtatttccgcgtga

SEQ ID NO: 59 - DNA, 22 bp, Artificial Sequence
  Synthetic oligonucleotide
ccatgaagcatgagaatgaggc

SEQ ID NO: 60 - DNA, 22 bp, Artificial Sequence
  Synthetic oligonucleotide
cttgttgacgactttctgttgc

SEQ ID NO: 61 - DNA, 22 bp, Artificial Sequence
  Synthetic oligonucleotide
aaagatgtgcttcgagatgtgt

SEQ ID NO: 62 - DNA, 23 bp, Artificial Sequence
  Synthetic oligonucleotide
cactttgtcagttaccaacgtca

SEQ ID NO: 63 - DNA, 23 bp, Artificial Sequence
  Synthetic oligonucleotide
cagtttgccccttttagtagtgc

SEQ ID NO: 64 - DNA, 20 bp, Artificial Sequence
  Synthetic oligonucleotide
ccagccgatactcgttcagc

SEQ ID NO: 65 - DNA, 20 bp, Artificial Sequence
  Synthetic oligonucleotide
ctgctggcgacaccaagaaa

SEQ ID NO: 66 - DNA, 19 bp, Artificial Sequence
  Synthetic oligonucleotide
cgtgttcgctctactgccc

SEQ ID NO: 67 - DNA, 21 bp, Artificial Sequence
  Synthetic oligonucleotide
cctgagtcccttctaccttcg

SEQ ID NO: 68 - DNA, 23 bp, Artificial Sequence
  Synthetic oligonucleotide
cacatctcccaacaccttaactt

SEQ ID NO: 69 - DNA, 21 bp, Artificial Sequence
  Synthetic oligonucleotide
caaacagaagaaccgtcccaa

SEQ ID NO: 70 - DNA, 21 bp, Artificial Sequence
  Synthetic oligonucleotide
tcacctcggaacaactgcaat

SEQ ID NO: 71 - DNA, 20 bp, Artificial Sequence
  Synthetic oligonucleotide
agcagggcaatcgtctgttc

SEQ ID NO: 72 - DNA, 21 bp, Artificial Sequence
  Synthetic oligonucleotide
caaggcccggttggtgtaata

SEQ ID NO: 73 - DNA, 23 bp, Artificial Sequence
  Synthetic oligonucleotide
ctggaagatggacgtactttgtc

SEQ ID NO: 74 - DNA, 22 bp, Artificial Sequence
  Synthetic oligonucleotide
cgacgaaggcgactaattttgc

SEQ ID NO: 75 - DNA, 19 bp, Artificial Sequence
  Synthetic oligonucleotide
tcgccgctgtccaagaaac

SEQ ID NO: 76 - DNA, 22 bp, Artificial Sequence
  Synthetic oligonucleotide
agtaaaggccctcgtctatgtc

SEQ ID NO: 77 - DNA, 22 bp, Artificial Sequence
  Synthetic oligonucleotide
ctcagcttaccttgagaactcg

SEQ ID NO: 78 - DNA, 22 bp, Artificial Sequence
  Synthetic oligonucleotide
ttctagcgcctttcttgttcat

SEQ ID NO: 79 - DNA, 20 bp, Artificial Sequence
  Synthetic oligonucleotide
gctcaatcggcactgggtat

SEQ ID NO: 80 - DNA, 21 bp, Artificial Sequence
  Synthetic oligonucleotide
acctgctactgccattccaac

SEQ ID NO: 81 - DNA, 21 bp, Artificial Sequence
  Synthetic oligonucleotide
ggctaccttactagctgctcg

SEQ ID NO: 82 - DNA, 21 bp, Artificial Sequence
  Synthetic oligonucleotide
gattggcgatgtaggacccag

(19) United States
(12) Patent Application Publication
(10) Pub. No.: US 2020/0377885 A1
(43) Pub. Date: Dec. 3, 2020

(54) COMPOSITIONS AND METHODS FOR THE GENERATION OF NEURONS AND USES THEREOF

(71) Applicant: Washington University, St. Louis, MO (US)

(72) Inventors: Andrew Yoo, St. Louis, MO (US); Daniel Abernathy, St. Louis, MO (US); Michelle Richner, St. Louis, MO (US); Matheus Victor, St. Louis, MO (US)

(21) Appl. No.: 16/636,791

(22) PCT Filed: Jul. 30, 2018
(86) PCT No.: PCT/US2018/044317

§ 371 Date: Feb. 5, 2020

Related U.S. Application Data
(60) Provisional application No. 62/541,858, filed on Aug. 7, 2017.
(60) Provisional application No. 62/562,222, filed on Sep. 22, 2017.
(60) Provisional application No. 62/598,368, filed on Dec. 13, 2017.

(51) Int. Cl. C12N 15/113; C40B 30/06; G01N 33/50; G01N 33/68

(52) U.S. Cl. CPC C12N 15/113; C40B 30/06; G01N 33/5058; C12N 2506/1307; C12N 2310/141; C12N 2501/65; G01N 33/6896

(57) ABSTRACT

Among the various aspects of the present disclosure is the provision of a method of generating a neuron. In some embodiments, the neuron is generated from an adult fibroblast cell comprising miR-9/9* and miR-124 (miR-9/9-124); and one or more transcription factors. In some embodiments, the transcription factors comprise ISL1 and LHX3 or CTIP2, DLX1, DLX2, and MYT1L (CDM).

### CROSS-REFERENCE TO RELATED APPLICATIONS

This application claims priority from U.S. Provisional Application Ser. No. 62/541,858 filed on 7 Aug. 2017, U.S. Provisional Application Ser. No. 62/562,222 filed on 22 Sep. 2017, and U.S. Provisional Application Ser. No. 62/541,858 filed on 14 Dec. 2017, each of which is incorporated herein by reference in its entirety.

### STATEMENT REGARDING FEDERALLY SPONSORED RESEARCH OR DEVELOPMENT

This invention was made with government support under grant number NS083372 awarded by National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to methods and compositions for generating neurons, conversion of fibroblasts, and treatment of motor neuron diseases.

### SUMMARY OF THE INVENTION

Among the various aspects of the present disclosure is the provision of a method of generating a neuron. In some embodiments, the neuron is generated from an adult fibroblast cell comprising miR-9/9 and miR-124(miR-9/9*-124); and one or more transcription factors. In some embodiments, the transcription factors comprise ISL1 and LHX3.

An aspect of the present disclosure provides for a method of generating a neuron from an adult somatic cell. In some embodiments, the method comprises (i) providing an adult somatic cell, optionally, a fibroblast; (ii) providing at least one miRNA to the somatic cell; or (iii) providing one or more transcription factors to the somatic cell, resulting in conversion of the somatic cell into a converted neuron. In some embodiments, the adult somatic cell is an adult human fibroblast of mesodermal origin. In some embodiments, the miRNA is selected from miR-9/9* and miR-124 (miR-9/9*-124). In some embodiments, the one or more transcription factors comprise: a motor neuron transcription factor comprising ISL1 and LHX3; or a striatal-enriched factor comprising CTIP2, DLX1, DLX2, and MYT1L (CDM); or optionally further comprise a neurogenic transcription factors comprising NeuroD2, ASCL1 and Myt1L (DAM). In some embodiments, the one or more transcription factors initiate conversion of a somatic cell toward a clinically relevant cell type. In some embodiments, the miRNA, optionally miR-9/9*-124, is expressed in the somatic cell by transduction; the one or more transcription factors, optionally a motor neuron transcription factor or a striatal-enriched factor, are expressed in the somatic cell by transduction; or the converted neuron is a motor neuron or a medium spiny neuron (MSN). In some embodiments, the miRNA, optionally miR-9/9*-124, or the one or more transcription factors, optionally a motor neuron transcription factor or a striatal-enriched factor, is expressed in the somatic cell by viral vector transduction, optionally lentivirus transduction. In some embodiments, a viral vector expresses miRNA, optionally miR-9/9*-124 and an anti-apoptotic gene, optionally, BCL-XL, beneficial for neuronal conversion, under a doxycycline-inducible promoter. In some embodiments, the miRNA or transcription factors are cloned into a lentiviral plasmid; a lentivirus is produced and the somatic cells are infected; the lentivirus genome comprises miRNA or one or more transcription factors and is transfected into the fibroblast genome, resulting in a transduced fibroblast cell; or the miRNA or transcription factors are stably expressed by the transduced fibroblast cell. In some embodiments, the miRNA or the one or more transcription factors are administered exogenously to the somatic cells. In some embodiments, the miRNA coordinates epigenetic and transcriptional changes resulting in neuronal cell fate conversion; induces a generic neuronal state characterized by loss of fibroblast identity, presence of a pan-neuronal gene expression program, and absence of subtype specificity; initiates subunit switching within BAF chromatin remodeling complexes while separately repressing neuronal cell-fate inhibitors REST, CO-REST, and SCP1: or alters expression of genes involved in DNA methylation, histone modifications, chromatin remodeling, and chromatin compaction. In some embodiments, the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron. In some embodiments, the converted neuron phenotypically resembles endogenous motor neurons when compared using immunostaining analysis or gene expression profiling; the converted neuron resembles endogenous motor neurons when compared using electrophysiological tests or co-culture tests; or the converted neuron retains donor age marks and positional information.

Another aspect of the present disclosure provides for a method of modeling a neurodegenerative disease. In some embodiments, the method comprises: (i) providing a fibroblast from a subject with a neurodegenerative disease; or (ii) providing miR-9/9* and miR-124 (miR-9/9*-124) or one or more transcription factors to the fibroblast. In some embodiments, the neurodegenerative disease, disorder, or condition is selected from one or more of the group consisting of: (i) a motor neuron disease; (ii) spinal cord injury (SCI); (iii) Amyotrophic Lateral Sclerosis (ALS) or Spinal Muscular Atrophy (SMA); or (iv) Huntington's Disease (HD) or Alzheimer's Disease (AD). In some embodiments, the transcription factors comprise striatal-enriched factors or motor neuron transcription factors. In some embodiments, the striatal-enriched factors comprise CTIP2, DLX1, DLX2, and MYT1L (CDM) or the motor neuron transcription factors comprise ISL1 and LHX3. An aspect of the present disclosure provides for a method of generating a Huntington's Disease (HD) cellular platform comprising: (i) providing adult fibroblasts from a subject with HD; and (ii) providing miR-9/9* and miR-124 (miR-9/9*-124) and CDM to the fibroblast; wherein providing miR-9/9*-124 and CDM to an adult fibroblast results in generation of HD-MSNs from adult fibroblasts (HD-FB). In some embodiments, the HD-MSNs exhibit an HD-associated phenotype selected from one or more of the group consisting of: formation of aberrant protein aggregates, mHTT-induced DNA damage, spontaneous degeneration over time in culture, decline in mitochondrial function, or CAG repeat lengths remain stable after neuronal conversion.

Another aspect of the present disclosure provides for a composition comprising a cell-conversion agent. In some embodiments, the composition comprises: (i) one or more micro RNA and one or more of a motor neuron transcription factor or a striatal-enriched factor; (ii) a viral vector comprising miR-9/9*-124; (iii) a viral vector comprising ISL1 and LHX3; or (iv) a viral vector comprising CDM. In some embodiments, the cell-conversion agent initiates lineage-specific neuronal reprogramming in an adult fibroblast and generating a human neuron subtype from an adult fibroblast.

Another aspect of the present disclosure provides for a method of screening a candidate drug for effectiveness in treating a neurodegenerative or motor neuron disease. In some embodiments, the method comprises: (i) providing a cellular platform, the cellular platform comprising a neuron generated from a fibroblast of a subject with a neurodegenerative or motor neuron disease: (ii) providing a candidate drug; (iii) contacting the candidate drug and the cellular platform; and (iv) assessing efficacy of the candidate drug. In some embodiments, the cellular platform comprises cells obtained from a subject with a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD). In some embodiments, the efficacy is evaluated by monitoring the neurons for reversal of electrical impairment.

Another aspect of the present disclosure provides for a method of screening therapeutic efficacy. In some embodiments, the method comprises: (i) providing a Huntington's Disease (HD) cellular platform comprising HD-MSNs derived from an adult human fibroblast; (ii) contacting the HD-MSNs with a therapeutic agent; or (iii) evaluating HD-MSN response to the therapeutic agent. In some embodiments, the therapeutic agent comprises a pharmacological factor or a genetic factor; or evaluating HD-MSN response comprises detecting levels of spontaneous cell death, stress-induced cell death, or electrophysiological properties.

Another aspect of the present disclosure provides for a method of treating a neurodegenerative disease disorder, or condition in a subject. In some embodiments, the method comprises: (i) administering an SP9 regulating agent to the subject; (ii) expressing SP9 in MSNs by cloning the cDNA of SP9 downstream of a human EF1α promoter in a viral vector.

Another aspect of the present disclosure provides for a method of screening compositions for an SP9 modulating agent. In some embodiments, the method comprises: obtaining cells from a subject; contacting the cells with a suspected SP9 modulating agent; or measuring the expression of SP9 on the cells.

An aspect of the present disclosure provides for a method of treating a neurodegenerative disease disorder, or condition in a subject. In some embodiments, the method comprises: (i) providing an adult fibroblast; (ii) providing miR-9/9* and miR-124 (miR-9/9*-124) and one or more transcription factors to the adult fibroblast; (iii) administering a cell-conversion agent composition comprising miR-9/9* and miR-124 (miR-9/9*-124) and one or more transcription factors, optionally, a motor neuron transcription factor comprising ISL1 and LHX3 to a fibroblast or a subject; or (iv) administering a converted neuron, optionally a motor neuron, to a subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1G is a series of illustrations, images, and graphs depicting the direct conversion of young and old primary adult human fibroblasts into neurons via miRNA overexpression. FIG. 1A is an illustration showing the experimental schema for miR-9/9*-124 mediated direct neuronal conversion. FIG. 1B is a series of images showing adult human fibroblasts ectopically expressing miR-9/9*-124 for 35 days immunostained for the pan-neuronal markers TUBB3, MAP2 and NEUN. Insets represent starting fibroblasts co-stained as negative controls. Scale bars=20 μm. FIG. 1C is a bar graph showing quantification of TUBB3, MAP2 and NEUN positive cells over total number of cells (DAPI). For TUBB3 and MAP2, only cells with processes at least three times the length of the soma were counted. For NEUN, only cells with proper nuclear localization were counted. Data are represented as mean±SEM. 22 Yr Female n=238 cells, 42 Yr Female, n=100 cells, 56 Yr Male n=171 cells, and 68 Yr Female n=216. FIG. 1D is a series of images demonstrating converted neurons display hallmark sodium channel (SCN1A), axonal initial segment (ANKG) (left) and synaptic vesicle (SV2) (right) staining patterns. Scale bars=20 μm. FIG. 1E is a series of representative traces of TTX-sensitive inward and potassium whole-cell currents. FIG. 1F is a series of repetitive AP waveforms in response to 500 ms current injections recorded from neurons converted in monoculture. FIG. 1G is a pie chart summary of firing patterns observed in 23 neurons recorded in current-damp mode (left) and representative waveforms within each firing pattern recorded (right). See also FIG. 2.

FIG. 2A-FIG. 2F (related to FIG. 1) is a series of illustrations, images and graphs demonstrating that the miRNA-mediated conversion of fibroblasts into the neuronal fate is stable. FIG. 2A is a detailed schematic of the miR-9/9*-124 direct conversion protocol. FIG. 2B is a combined line graph plotting the current (1)-voltage (V) relationship for every neuron recorded. FIG. 2C is a column graph with points showing the tabulated values of miN resting membrane potentials. FIG. 2D is a column graph with points showing the tabulated values of miN capacitance values. FIG. 2E is a line graph showing the gap-free recording of miN resting membrane potential. FIG. 2F is a drawing illustrating how miR-9/9*-124 was ectopically expressed under a doxycycline (DOX) inducible promoter in 22-year-old human fibroblasts for 30 days then DOX was removed from the media and cells were cultured for an additional 30 days. FIG. 2F also includes microscopy co-staining images demonstrating that the adoption of neuronal fate was stable. Cells immunostained for the pan-neuronal markers MAP2, NeuN, TUBB3 and NCAM. To assay if cells remained post-mitotic, cells were stained with the proliferative marker ki-67. Scale bar=20 μm. All data are represented as mean±SEM.

FIG. 3A-FIG. 3D is a series of graphs and gene expression profiling which reveal that pan-neuronal identity can be induced by miRNAs alone. FIG. 3A is a scatterplot showing the genome wide expression analysis of miNs and starting fibroblasts by RNA-seq. This plot shows the relationship between average gene expression (logCPM) and log fold-change of miNs compared to fibroblasts. A selection of pan-neuronal and fibroblast-specific genes are highlighted in black text. Blue=fold change<−2 log₂ p<0.01 (more abundant in fibroblasts), grey=fold change >-2 log₂<2 log₂ p>0.01, and red=fold change>2 log₂ p<0.01 (more abundant in miNs). FDR<0.01. FIG. 3B shows representative genome browser snapshots demonstrating increased expression for a pan-neuronal gene (NEFL), loss of fibroblast gene expression (S100A4), and absence of neuronal subtype marker gene expression (MNX1, motor neuron marker; DARPP-32, medium spiny neuron marker). FIG. 3C includes a set of bar graphs which list the gene ontology (GO) terms associated with genes upregulated in miNs (red) and GO terms associated with genes downregulated in miNs (blue). The heat maps to the right show genes that fall within top GO categories listed (top to bottom) in order of lowest to highest p-value. FIG. 3D is a volcano plot representing chromatin remodeling genes differentially expressed between fibroblasts and miNs. Blue dot, abs(logFC)>2 and p<0.01, red dot, abs(logFC)>1 and p<0.01, grey dot, no significant difference. See also FIG. 4.

FIG. 4 (related to FIG. 3) is a series of heat maps that demonstrate the widespread expression changes in epigenetic modifiers (a subset of proteins that recognize or modify distinct parts of the epigenome) observed between miNs and fibroblasts during reprogramming.

FIG. 5A-FIG. 5G is a series of illustrations, graphs and traces that show how miR-9/9*-124 alter DNA methylation at neuronal loci. FIG. 5A is a schematic of sample collection during miR-9/9*-124-mediated neuronal reprogramming for DNA methylation studies. Human fibroblasts were transduced with virus expressing miR-9/9*-124 or a non-specific (NS) control (Ctrl) virus at day 0. Samples were collected at day 10, day 20, and day 30. FIG. 5B is a bicusteng analysis of DMRs. Heatmaps based on MeDIP-seq RPKM (left) and MRE-seq RPKM (right) show overlapping DMRs at days 20 and day 30. FIG. 5C is a line graph quantifying DMRs at multiple q-value cutoffs (q<5e-2 in red; q<Se-3 in yellow; q<Se-4 in purple) across all time points: miN 10 (miN day 10 vs. Ctrl day 10), miN 20 (miN day 20 vs. Ctrl day 20), and miN 30 (miN day 30 vs. Ctrl day 30). FIG. 5D is a bar graph showing the tissue development enrichment of the top overlapping DMRs at day 20 and day 30; the DMRs were enriched for neuronal tissue development terms, specifically at TS15 (˜E9/10 in mouse development). FIG. 5E is a series of WashU Epigenome Browser screenshots of two DMRs: FBXO31 (left) and MIRLET7BHG (right) loci are shown with MeDIP-seq tracks (red; top), MRE-seq tracks (green; middle), and DMR positions (purple; bottom). FIG. 5F is a pie chart showing the genomic distribution of differentially methylated and demethylated regions. FIG. 5G is a set of bar graphs showing the functional enrichment of the top demethylated and upregulated (red; left) or the top methylated and downregulated (blue; right) DMRs overlapping at day 20 and day 30 compared with RNA-seq at day 30.

FIG. 6A-FIG. 6I is a series of graphs, heat maps and traces that demonstrates how miR-9/9*-124 can globally change chromatin accessibility. FIG. 6A is a two dimensional correlation plot of samples. Pearson's correlation coefficient is as follows: 0.90 for Ctrl D10 (or FIB); 0.83 for miNs D10 (D10); 0.90 for miNs D20 (D20). FIG. 6B is a pie chart showing the proportion of differential peaks and total peaks. Differential peaks were obtained by combining all significant peaks (Ctrl D10 vs miNs D10, miNs D10 vs miNs D20). FIG. 6C is a series of heatmaps showing signal intensity in open and close chromatin peaks across all time points. All open and closed chromatin regions were ranked according to maximum intensity across all samples. FIG. 6D is a pie chart showing the genomic distribution of open and closed chromatin regions. FIG. 6E is a set of bar graphs showing a comparison of GO terms for genes with open chromatin regions at promoters in miNs at day 10 and 20, but closed chromatin regions in fibroblasts. FIG. 6F is a series of heatmaps showing gene expression levels for DEGs positively correlated with ATAC-seq signal intensity in their promoter regions. Signal intensity is based on normalized counts per million (CPM) values. FIG. 6G is a set of bar graphs displaying the top GO terms associated with DEGs which correlate with ATAC-seq signal intensity in promoter regions. FIG. 6H is a series of heatmaps showing signal intensity in the open (accessible) and closed (inaccessible) chromatin regions that overlapped with histone-marked regions of fibroblasts. FIG. 6I is a set of integrated genomics viewer (IGV) screenshots showing two different examples of ATAC-seq and RNA-seq integration. The first image shows an example of ATAC-seq and RNA-seq peaks within the pan-neuronal gene MAP2. The second image shows an example of ATAC-seq peaks within a subtype-specific locus without gene expression changes: MNX1. See also FIG. 7 and FIG. 8.

FIG. 7A-FIG. 7D (related to FIG. 6) is a series of bar graphs showing that pre-existing heterochromatic neuronal loci open in response to miR-9/9*-124 expression. FIG. 7A is a set of bar graphs showing the top GO terms associated with promoter regions that close during reprogramming from fibroblasts to miNs. FIG. 7B is a bar graph showing that the closed regions in fibroblasts marked by H3K9me3 that open during neuronal reprogramming are enriched for neuronal GO terms. FIG. 7C is a bar graph showing that the closed regions in fibroblasts marked by H3K27me3 that open during reprogramming are also enriched for neuronal GO terms. FIG. 7D is a bar graph that displays that pre-existing distal H3K27ac and H3K4me1 marks within fibroblasts that close during neuronal reprogramming show GO terms related to general biological processes.

FIG. 7E is a genome browser snapshots demonstrating closing and loss of fibroblast gene expression (ECM1, MFAP5, and VIM).

FIG. 7F is a genome browser snapshots demonstrating neither opening or activation of gain of progenitor genes (SOX2, OLIG2 and ASCL1).

FIG. 7G is a genome browser snapshots demonstrating neuronal subtype gene loci that open, but do not show gene expression changes (GAD2 and GABRA2, GABAergic markers; TH, dopaminergic neuron marker).

FIG. 8A-FIG. 8C (related to FIG. 6) is a series of images that show how combining ATAC and RNA-seq can reveals a subtype poised neurogenic state.

FIG. 8A is a series of genome browser snapshots demonstrating the opening and activation of gain of pan-neuronal genes (SNAP25 and MAP2). FIG. 8B is a series of genome browser snapshots that display the closing and loss of fibroblast gene expression (CORO1C, MMP2, and VIM). FIG. 8C is a series of genome browser snapshots that show how neuronal subtype gene loci open but do not show gene expression changes (GAD2 and GABRA2, GABAergic markers; TH, dopaminergic neuron marker).

FIG. 9A-FIG. 9C (related to FIG. 10) is a series of images and graphs that show the identification of transcription factors for defining motor neuron specific conversion. FIG. 9A shows a list of candidate motor neuron transcription factors and factor combinations that co-expressed with miR-9/9*-124 in human fibroblasts. FIG. 9B is a set of microscopy images showing immunocytochemistry of adult human fibroblasts overexpressing a non-specific miRNA (miR-NS) and ISL1/LHX3 (left) or miR-9/9*-124 and ISL1/LHX3 (right) for 35 days. These images demonstrate the necessity of miR-9/9*-124 for opening the neurogenic potential of human fibroblasts. Scale bar=20 μm. FIG. 9C is a series of images and line graphs. These additional representative inward/outward whole-cell currents and repetitive AP waveforms were generated from whole cell patch damp recordings of Moto-miNs. Images show the patch damped cells.

FIG. 10A-FIG. 10F is a series of illustrations, images and graphs showing how miRNA-induced neuronal competence enables motor neuron transcription factors, ISL1 and LHX3, to determine motor neuron Identity. FIG. 10A is a schematic illustrating a neuronal induction paradigm using miR-9/9*-124 plus ISL1 and LHX3. FIG. 10B is a series of images showing representative immunohistochemistry for pan-neuronal markers in neurons generated from fibroblasts through 35 days of ectopic miR-9/9*-124, ISL1, and LHX3 co-expression. Fibroblasts were isolated from a 22 year old female donor. Scale bars=20 μm. FIG. 10C is a bar graph showing the quantification of 4 independent primary human fibroblast lines from both male and female donors stained with TUBB3, MAP2 and NCAM. Percentages represent total number of positive cells over all cells (DAPI) and are represented as mean±SEM. Cells analyzed: 22 yr old N=TUBB3 325, MAP2 219, NCAM 275; 42 yr old N=TUBB3 304, MAP2 236, NCAM 129; 56 yr old N=TUBB3 275, MAP2 279, NCAM 213; 68 yr old N=TUBB3 282, MAP2 234, NCAM 190. FIG. 10D is a series of images displaying the expression and correct localization of motor neuron markers in neurons converted by miR-9/9*-124 and ISL1/LHX3 as demonstrated by immunohistochemistry. MNXI (top), CHAT (middle), and SMI-32 (bottom). Scale bars=20 μm. FIG. 10E is a bar graph showing a quantification of FIG. 10D, representing the total percentage of MNX1, CHAT, and SMI-32-positive cells over TUBB3-positive cells. Data are represented as mean±SEM. Cells analyzed: 22 yr old N=MNX1 256, CHAT 256, SMI-32 113; 42 yr old N=MNX1 151, CHAT 151, SMI-32 283; 56 yr old N=MNX1 207, CHAT 207, SMI-32 174; 68 yr old N=MNX1 151, CHAT 151, SMI-32 96. FIG. 10F includes a schematic illustrating how after 30 days of neuronal conversion by ectopic miR-9/9*-124 expression, doxycycline was removed and cells were cultured for an additional 30 days. Immunocytochemistry showing motor neurons produced by miR-9/9*-124 plus ISL1 and LHX3 (Moto-miNs) remain Ki-67 negative (2nd panel), retain expression and localization of the neuronal proteins TUBB3, NEUN, and MAP2 (2nd and 3rd panel), and express the motor neuron proteins MNX1 and CHAT (4th and 5th panel). Scale bars=20 μm. See also FIG. 9.

FIG. 11A-FIG. 11L is a series of traces, graphs and images that show the functional properties and gene expression profile of Moto-miNs. FIG. 11A is a line graph showing representative traces of inward sodium and outward potassium whole-cell currents. FIG. 11B is a trace of repetitive AP waveforms in response to 500 ms current injections recorded from Moto-miNs converted in monoculture. FIG. 11C is a series of images showing the representative waveforms of a single Moto-miN at increasing current injections. FIG. 11D is a set of pie charts summarizing the firing patterns observed in Moto-miNs converted from both old and young donors. The Moto-miNs from a 68-year-old donor exhibited 80% multiple fire (N=20), and the Moto-miNs from a 22-year-old donor exhibited 74% multiple fire (N=25). FIG. 11E is a representative trace of the spontaneous firing activity which observed in a small percentage of Moto-miNs (3 out of 20). FIG. 11F is a combined line plot showing the current (1)-voltage (V) relationship for every Moto-miN recorded. Data are represented as mean±SEM. FIG. 11G is a column graph with data points showing how Moto-miNs converted from both young and old donors are hyperpolarized, demonstrating mean resting membrane potentials of −67.2 mV and −72.8 mV, respectively. Data are represented as mean±SEM. FIG. 11H is a set of images showing the staining of Moto-miNs cultured with differentiated human myotubes. Moto-miNs were labeled with synapsin-EGFP via viral transduction, and then plated onto human myotubes. Myotube only cultures did not have a-Bungarotoxin-594 (red) puncta (top left inset). Scale bar=20 μm. FIG. 11I is a set of scatterplots comparing the mean gene expression of starting fibroblasts from a 22 year old donor (y-axis) and miNs generated from the same individual (x-axis). Left plot highlights a selection of pan-neuronal and fibroblast-specific genes in green text. Blue=log₂FC<−2.5 and p<0.05, (more abundant in fibroblasts) grey=log₂FC>-2.5 and <2.5, p>0.05 (no significant difference), and red=log₂FC>2.5 and p<0.05 (more abundant in miNs). The right plot is a scatterplot comparing the mean gene expression of starting fibroblasts from a 22-year old donor (y-axis) and Moto-miNs generated from the same individual (x-axis). Plot highlights a selection of pan-neuronal and motor neuron-specific genes in green text. Blue=log₂FC<−2.5 and p<0.05, (more abundant in miNs) grey=log₂FC>-2.5 and <2.5 p>0.05 (no significant difference), and red=log₂FC>2.5 and p<0.05 (more abundant in Moto-miNs). FIG. 11J is a bar graph displaying that Moto-miNs generated from multiple donors have lower mRNA levels for fibroblast genes and increase expression of motor neuron-specific genes. Moto-miNs were analyzed by qRT-PCR 35 days post-transduction. Human spinal cord RNA served as a positive control (normalized to 42 yr fibroblasts, ΔΔct method). Data are represented as mean±SEM. FIG. 11K is a bar graph showing that miR-9/9*-124 and ISL1/LHX3 activate the expression of the motor neuron specific miRNA, miR-218. RNA was isolated from fibroblasts and Moto-miNs 35 days post-transduction and analyzed by qRT-PCR. Data are represented as mean SEM. FIG. 11L is scatterplot showing that Moto-miNs retain donor fibroblast HOX gene expression pattern as demonstrated by qRT-PCR. Act method. Data represent Act values for each biological replicate (3 separate Moto-miN conversions). See also FIG. 12 and FIG. 13.

FIG. 12A-FIG. 12C (related to FIG. 11) is a series of graphs and images showing that the addition of ISL1/LHX3 increases functional maturity and generates a motor neuron transcriptional network. FIG. 12A is a dot plot showing the pPeak inward current measured during voltage clamp mode of miNs and Moto-miNs; these data reveal increased peak inward current in Moto-miNs (−3,189 pA±214 pA) compared to miNs (−919 pA±113 pA). Data are represented as mean±SEM. FIG. 12B is an image of an analysis using the Cell Type-specific Enrichment Analysis (CSEA) tool. This analysis reveals that the top 100 most significantly expressed genes in miNs do not enrich for defined neuronal subtypes (top), while the top 100 most significantly expressed genes in Moto-miNs are enriched in cholinergic motor neurons in the brain stem and spinal cord. FIG. 12C is a set of scatterplots showing HOX gene expression analysis by qRT-PCR in 42 year old female and 56 year old male donor fibroblasts before and after conversion confirms that Moto-miNs retain donor fibroblast HOX gene expression patterns. Data represent Act values for each biological replicate (3 separate Moto-miN conversions).

FIG. 13 (related to FIG. 11) is a diagram and a heatmap showing direct comparison of moto-miN transcriptome to in vivo mouse motor neurons by translating ribosomal affinity purification (TRAP) sequencing. On the left is a Venn Diagram depicting the number of ISL1/LHX3 ChiP-seq peaks identified by Mazzoni et al. during ISL1/LHX3 directed ES to motor neuron differentiation (3,486) and genes enriched in Moto-miN transcriptome (775). On the right is a heatmap showing that the overlapping activated genes (323) include hallmark motor neuron markers.

FIG. 14A-FIG. 14C is a series of illustrations, graphs and heatmaps showing how the direct comparison of Moto-miN transcriptome to in vivo mouse motor neurons by Translating Ribosomal Affinity Purification (TRAP) sequencing. FIG. 14A is a schematic of the TRAP-Seq strategy used to identify transcripts in all neurons (SNAP-25 genetic driver) and motor neurons (CHAT genetic driver) in mouse spinal cord. TRAP is a method to precipitate actively translated mRNA bound to ribosomes using an antibody to EGFP-L10A. FIG. 14B is a scatterplot showing the pairwise comparisons between mean expression values in CHAT IP v. Pre-IP (first) and CHAT IP v. SNAP25 IP (second). Differentially expressed genes are shown in red (logFC>1 and p<0.05) and blue (logFC<-1 and p<0.05). FIG. 14C is a set of heat maps showing example mean expression values of overlapping genes between human (Moto-miNs versus miNs) and mouse (all spinal cord neurons SNAP25-TRAP) and motor neurons (ChAT-TRAP) datasets. FIG. 15A-FIG. 15C is a series of images and graphs showing that Huntington's Disease (HD) patient fibroblasts can be directly reprogrammed into medium spiny neurons (MSNs). Fibroblasts of three HD patients (with mHTT expansions of 40, 43 and 44 CAGs) and their respective age- and sex-matched controls (CAG sizes of 19, 17 and 18) were reprogrammed into MSNs with miR-9/9-124+CDM. FIG. 15A is a series of immunofluorescence images displaying reprogrammed HD.40 at post-induction day (PID) 30 immunostained with TUBB3, and HD.44 with TUBB3, NeuN, MAP2, DARPP-32 and GABA. FIG. 15B shows images of all three pairs of cells analyzed immunostained for GABA and DARPP-32. Scale bar 50 μm. FIG. 15C is a series of bar graphs quantifying TUBB3, GABA and DARPP-32-positive cells at PID 30; n=1,000 cells from 3 samples for each pair. Mean and S.D. Unpaired t-test; p-value >0.05 df=4.

FIG. 16A-FIG. 16E are a series of images and graphs showing HD fibroblasts can be successfully reprogrammed by miR-9/9-124+CDM independent of donors age or CAG-repeat size and DARPP-32 (Santa Cruz-H62 Clone) antibody shows specificity to striatum in both mouse and human striatal sections. FIG. 16A is a series of images and traces showing electrophysiolocal properties were analyzed in monoculture free of rat or mouse primary glia/neurons. HD.59 was analyzed at PID 23 while HD.180 was analyzed at PID 30. FIG. 16B is shows RNA-seq analysis at PID 32 of adult control and HD patient fibroblasts reprogrammed with miR-9/9-124+CDM reveals expression of the full length DARPP-32 transcript. FIG. 16C shows immunostaining with an additional anti-DARPP-32 antibody (abcam; ab40801) also produced positive cells, although with less intensity (Cells depicted were reprogrammed from GM04855 fibroblasts). FIG. 16D shows further validation of the up-regulation of DARPP-32 by qPCR with DARPP-32 specific probes. FIG. 16E are images showing DARPP-32 (Santa Cruz-H62 Clone) showing specificity to MSNs in the striatum of an adult mouse brain as seen by immunohistochemistry analysis, where only the striatum is labeled (shown in red) in a brain coronal section. Similarly, immunohistochemistry performed in a human postmortem brain section of globus pallidus with putamen of a 89 year old healthy female obtained from NIH NeuroBioBank shows antibody labeling specificity to the striatum.

FIG. 17 shows CAG-Sizing of primary fibroblasts and microRNA-derived MSNs. CAG repeat analysis confirmed HTT mutation and number of CAGs in cell lines mainly used in this study. After several passages in culture and subsequent reprogramming into MSNs by miR-9/9*-124+CDM for three weeks, CAG size was stable (for each group non-transduced fibroblasts are shown on the left and reprogrammed MSNs on the right).

FIG. 18A-FIG. 18D are a series of traces and graphs showing electrophysiological analysis of HD and control MSNs. pSynapsin-tRFP labeled reprogrammed cells were plated onto primary rat glial cultures and cultured for 28 days. (HD; HD.47, Ctrl; Ctrl. 16). FIG. 18A are representative traces from Ctrl-MSNs in gray (FIG. 18B) and traces from HD-MSNs in blue. FIG. 18A-FIG. 18B are voltage-clamp recordings of evoked action potentials (APs) and inset with progressive current-injection steps; Current-damp recordings of inward and outward currents and inset of sodium currents; Spontaneous firing of APs; Ramp protocol to determine AP threshold. FIG. 18C are a series of whisker plots showing all properties measured were quantified and found to not differ significantly (Student's t-test). FIG. 18D are a series of Venn diagram of recorded cells showing increased firing complexity in HD cells. All reprogrammed cells in both groups fired APs. Mean±s.d.; n=10 HD MSNs and 12 control MSNs.

FIG. 19A-FIG. 19G are a series of images, traces, graphs, and tables showing reprogramming HD patient fibroblasts generates functional neurons. FIG. 19A is a series of images showing GFP labeled Ctrl-MSNs and tRFP labeled HD-MSNs (pseudo-colored gray) co-cultured and seeded atop rat primary neural cells for whole-cell recording. FIG. 19B shows representative traces from current-damp recordings of Ctrl-MSNs (green traces) at PID 35, and (FIG. 19C), HD-MSNs (gray traces); Ctd-MSNs and HD-MSNs displayed similar firing patterns, with HD-MSNs having a greater percentage of cells that fired multiple action potentials. Inset display single trace at increasing stimulus steps and total number of cells that fired single or multiple action potentials. Voltage-clamp recordings demonstrate inward sodium and outward calcium currents typical of neurons. FIG. 19D shows HD-MSNs displayed spontaneous action potentials. FIG. 19E shows I-V curve. n=11 from 3 control samples; n=13 from 3 HD samples. Evoked response at 90 mV in HD-MSNs is significantly higher. One-way ANOVA (F39,440=24.21 P<0.001) with post hoc Tukey's test; **p=0.0068; Mean±S.D. FIG. 19F shows the analysis of passive membrane properties. n=3 averages for each line, totaling 18 controls and 20 HD-MSNs. Mean±s.e.m. Unpaired t-test; p-value >0.05 df=4 Scale bar in a, 10 μm. FIG. 19G is a table showing all recorded properties during electrophysiological analysis displayed for each reprogrammed line.

FIG. 20A-FIG. 20D show HD-MSNs properly acquire striatal cell fate identity and display differentially expressed genes. Analysis of fibroblast- and MSN-specific genes at PID 32 in HD.40 and HD.43 reprogrammed MSNs, including Ctrl-MSNs and respective fibroblasts, as well as additional analysis of a set of 7 HD and 5 Ctrl-MSNs by RNA-seq. FIG. 20A is a heat map representation of average expression values at PID 32 for 25 fibroblast-enriched genes and 48 MSN-enriched genes including CDM factors. n=2 biological replicates per sample of 2 HD- and Ctrl-MSNs and their corresponding fibroblasts. FIG. 20B shows a principal component analysis of gene expression data for 5 controls and 7 HD-MSNs samples analyzed at PID 32. n=2 replicates per sample. FIG. 20C shows a pairwise comparison of HD-MSNs and Ctrl-MSNs show many distinct genes differentially expressed in HD-MSNs with FDR<0.01. Mapped reads are displayed in log 2 counts per million (CPM) and fold-change in HD-MSNs expression displayed in gray-blue color gradient, with upregulated genes shown in gray and downregulated genes in blue. FIG. 20D depicts a gene ontology (GO) analysis of differentially expressed genes reveals many critical cellular processes, including a significant enrichment of genes associated with HD. Further GO analysis of these HD-related genes points to dysfunction in neurophysiological processes.

FIG. 21 depicts a collection of Huntington's disease-associated genes differentially expressed in HD-MSNs. FIG. 21 shows ingenuity pathway analysis (IPA) of differentially expressed genes identified in RNA-seq studies uncovered many genes that have been experimentally associated with HD. Genes shown in subcellular organization. Red genes are upregulated while green genes are downregulated in HD-MSNs, with darker colors representing higher expression levels.

FIG. 22A-FIG. 22I are a series of images and graphs showing mutant HTT aggregates in HD-MSNs. FIG. 22A-FIG. 22C are images showing HTT aggregation is not present in HD fibroblasts or Ctrl-MSNs but is detectable in HD-MSNs. Analysis at PID 30 by EM48 antibody. FIG. 22D are images showing HD-MSNs contain both cytoplasmic (arrowheads), and intranuclear indusions (arrow) detection by EM48 antibody at PID 30. FIG. 22E is a bar graph showing the quantification of percentage of Ctrl- and HD-MSNs displaying inclusion bodies (IBs) by MW8 antibody at PID 30; Mean±s.e.m.; *P<0.05 by Students t-test; t=3.787, df=4; n=averages of 400 cells from 3 independent HD patients and controls. FIG. 22F is a series of images showing co-localization of EM48 (red) with Ubiquitin (green). FIG. 22G-FIG. 22H are a series of images showing HD-MSNs on μ-dishes immunolabeled with HTT by MW8 conjugated to fluoronanogold reveal intranuclear inclusions by transmission electron microscopy (TEM). FIG. 22I is a series of images showing ultrastructural analysis also detected mutant HTT inside double membrane vesicles (arrow head) resembling autophagosomes. Immunostaining for the autophagosome marker LC3-II confirmed colocalization with HTT (MW8) at PID 30. Scale bars: 10 μm, except for TEM panel of FIG. 22I which is 100 nm.

FIG. 23A-FIG. 23G is a series of illustrations, images, and bar graphs showing HD fibroblasts do not exhibit inclusion bodies, even upon cellular insults. FIG. 23A shows fibroblasts induced to age in vitro by serial passaging (18 times), forced to exit cell cycle by contact inhibition, and then cultured for an additional 7 weeks, do not exhibit indusion bodies. FIG. 23B show images of Ctrl (Ctrl. 19) or HD fibroblasts (HD.40) challenged with 1 mM H₂O₂ to induce oxidative stress do not exhibit inclusion bodies. FIG. 23C is an illustration and an image of HD.40 fibroblasts transduced with CDM and a non-specific microRNA (miR-N.S.) to mimic reprogramming conditions but not neuronal induction, do not form inclusion bodies. The formation of inclusion bodies is present in all three lines reprogrammed from HD patients. FIG. 23D shows images of all three HD MSNs lines examined exhibit aggregated HTT inclusions (IBs) at post-induction day 30 (PID) analyzed by MW8 immunostaining. FIG. 23E is a bar graph showing quantification with EM48 yields similar number of cells displaying inclusions to MW8 staining; Student's t-test, n=4 biological replicates per group; ***p-value=0.0003 df=6. FIG. 23F images show the appearance of aggregated cytoplasmic HTT protein in HD.40 MSNs is detected as early as PID 14. By PID 21 HTT inclusions are numerous and after PID 28, inclusions are bigger and more defined, with little to no granules. Quantification of inclusion formation shows significant changes by PID 14; n=3 biological replicates with each sample containing approximately 100 cells; Mean±s.e.m. One-Way ANOVA (F6,17=65.47, P<0.0001) with post hoc Tukey's test.; ***P<0.001; n.s. not significant. FIG. 23F bar graph shows time course analysis with Ctrd. 20 and HD.42 for the appearance of oxidative DNA damage phenotype by 80H-dG staining. Significant differences between controls and HD MSNs were detected as early as PID 20, and continue to augment with time in culture. One-Way ANOVA (F9,190=30.86, P<0.0001) with post hoc Tukey's test; **P<0.001; *P<0.01; n.s.=not significant. n=20 cells per time point for each line. Mean±s.d.

FIG. 24A-FIG. 24F are a series of images and bar graphs showing ultrastructural analysis of HD-MSNs. FIG. 24A are images of immunogold labeling of HTT in HD-MSNs shows fibrillar-like structures. FIG. 24B is an image showing immunogold labeling of HTT in HD.40-MSNs at PID 21 is prominent within single and double-membrane autophagosome-like structures (black arrowheads), as well as accumulated as non-membrane bound cytoplasmic structures (red arrowheads). In addition there is marked presence of lipofuscin granules which are known to accumulate with aging (labeled with an asterisk) and quantified in FIG. 24C, for 3 independent control and HD lines. Student's t-test, n=average of all visible lipofuscins in 10 cells per line; *=p-value=0.0172 t=3.925 df=4. FIG. 24D is an image showing greater magnification of red arrows in FIG. 24B, where fibrilar-like structures can be seen. FIG. 24E is an image showing the ultrastructural analysis in HD.40 was also marked by mitophagy (left), accumulation of lipid droplets (middle) and swollen mitochondria typical of apoptotic cells (right): N=nucleus. FIG. 24F are images showing colocalization of HTT (EM48) and the autophagosome marker LC3 in additional HD lines.

FIG. 25A-FIG. 25C show the biochemical analysis of mutant HTT expression in reprogrammed MSNs. FIG. 25A shows the four samples used for westem blotting, all reprogrammed with miR-9/9*124+CDM and lysed for protein extraction at post-infection day (PID) 28. FIG. 25B shows anti-huntingtin monoclonal antibody MW1 specifically binds to the polyglutamine domain of HTT exon 1 and therefore recognizes expanded polyglutamine while showing no detectable binding to normal HTT. Our analysis confirms the expression of soluble mutant HTT in MSNs reprogrammed from primary fibroblasts samples from HD patients. FIG. 25C shows, unlike MW1, the monoclonal anti-huntingtin antibody MW8 recognizes amino acids 83-90 near the c terminus of exon 1 of HTT and specifically recognizes aggregated forms of mutant HTT. Our analysis with MW8 reveals detectable levels of insoluble aggregated HTT in HD-MSNs. Smaller proteins detected are likely breakdown products of HTT, or unrelated polyglutamine-containing proteins (Ko et al., Brain Research Bulletin 2001).

FIG. 26A-FIG. 26J are a series of images, illustrations, and graphs showing proteostasis collapses in directly reprogrammed MSNs but is spared in cells derived from iPSCs. FIG. 26A shows a schematic of the derivation of HD-MSNs from adult fibroblasts (HD-FB) versus embryonic fibroblasts (HD-HEFs). heMSNs: MSNs reprogrammed from HEFs. OSKM: Oct3/4, Sox2, Klf4, c-Myc. FIG. 26B are images of vimentin (VIM)- and fibronectin (FN)-positive HD.40-HEFs and HD.40-FB. FIG. 26C show images of HD.50-MSNs and HD.50-heMSNs analyzed for neuronal and MSN markers, and mutant HTT aggregates (MW8). FIG. 26D is a bar graph of the quantification of inclusion bodies (IBs); n=Average of 400 cells from 3 biological replicates; One-Way ANOVA (F2,6=18.67, P<0.0027) with post hoc Tukey's test. Scale bar in FIG. 26B, 100 μm; and in FIG. 26C, 10 μm. FIG. 26E shows live imaging in HD.40-FB and HD.40-HEFs expressing 23 or 74 polyglutamine (Q) repeats fused to GFP; scale bar 50 μm. Arrowheads mark IBs. FIG. 26F is a bar graph showing quantification of IBs post-transfection. n=average of 30 cells in each group for 3 independent experiments; One-Way ANOVA (F5,12=228.7, P<0.0001) with post hoc Tukey's test. FIG. 26G is a bar graphs showing treatment of HEFs with 5 μM of lactacystin induces IBs; n=average of 30 cells for 3 independent experiments; Student's t-test; df=4 (FIG. 26H-FIG. 26I), 20 seconds proteasome activity measured by cleavage of fluorogenic peptide LLVY-AMC for 1 hour; n=3 samples from each group; One-Way ANOVA (F4,10=282.3, P<0.0001) with post hoc Tukey's test. FIG. 26J shows an image of describing the microarray analysis of MSNs from neonatal or older healthy individuals shows reduction in ubiquitin proteasome system gene expression with age. ***P<0.001; **P<0.01; n.s.=not significant.; Mean±s.e.m.

FIG. 27A-FIG. 27H are a series if illustrations, images, and graphs showing adult HD fibroblasts can be induced to pluripotency and rederived to human embryonic fibroblasts (HEFs). FIG. 27A is a schematic of HEF derivation. FIG. 27B are images showing cells transduced with OCT4, SOX2, KLF4, or c-MYC (OSKM) express stem cell markers, FIG. 27C, and retain a normal karyotype. FIG. 27D are images showing induced pluripotent stem cells (iPSCs) can be differentiated into HEFs by addition of 20% fetal bovine serum (FBS) to culture media and passaging at least three times. FIG. 27E shows CAG sizing confirms that HEFs retain repeat number. FIG. 27F shows LAP2a levels are restored in HEFs; n=1,000 cells from 3 independent experiments; Students t-test p-value=0.0230; df=4 FIG. 27G shows MSNs directly converted from HD.40 FBs and HEFs express TUBB3, while heMSNs are nearly devoid of mHTT aggregates (MW8) at PID 21. FIG. 27H shows quantification of mHTT aggregates in MSNs versus heMSNs of HD.50 in comparison to Ctrl. 17; One-way ANOVA followed by Tukey's test (F2,6=21.85); Mean±s.e.m; **P<0.01; *P<0.05.

FIG. 28 is a bar graph showing induction of pluripotency alters expression of ubiquitin-proteasome system (UPS)-related genes. HD.40 fibroblasts (HD-FB) and two iPSC clones derived from HD.40 fibroblasts were differentiated into embryonic fibroblasts (HD-HEF.1 and HD-HEF.2) and analyzed by qPCR for the expression of UPS-related genes. HD-HEFs have higher expression of many UPS genes. Mean±s.e.m.; One-Way ANOVA (F50,102=20.17, P<0.0001) with Holm-Sidak correction. n=3 samples per group. ***P<0.001; **P<0.01; *P<0.05. Only significant changes are marked, and represents p-values for consistent for both HD-FB versus HD-HEF.1 or HD-HEF.2 tests. The expression of ATG12 was only significantly different in HEF2.

FIG. 29A-FIG. 29L are a series of images and graph showing DNA damage and degeneration in HD-MSNs. FIG. 29A-FIG. 29B show HD-MSNs have increased oxidative DNA damage by 8-OHdG immunostaining (F3,8=13.5, P=0.0016); n=averages from 70 cells from 3 independent HD and control lines; and FIG. 29C-FIG. 29D shows increased doubled stranded breaks detected by 53BP1 immunostaining (F3,8=20.68, P=0.0004); n=averages from 100 cells from 3 independent HD and control lines; FIG. 29E-FIG. 29F demonstrate single-cell gel electrophoresis (comet assay) detected significantly more double-stranded DNA breaks (F3,8=7.329, P=0.0111) in HD-MSNs than controls or fibroblasts; n=averages from 20 cells from 3 independent HD and control lines. FIG. 29G shows representative images of SYTOX green stain. FIG. 29H shows the quantification of SYTOX-positive cells over the nuclear dye Hoescht at each time point (F7,16=36.71, P<0.0001); n=averages of 6,000 cells for each time point from 3 independent HD and control lines. Solid lines represent the average while each line is shown separately as a dotted line. FIG. 29I shows AAV-mediated shRNA knockdown of HTT at PID 14 of reprogramming HD-MSNs attenuates DNA damage at PID 35; AAV non-specific (ns) shRNA used as control for viral infectivity; 8-OHdG: n=50 cells per group (F3,196=16.46, P<0.0001); 53BP1: n=averages of 100 cells per group from 3 independent experiments (F3,8=35.1, P<0.0001). FIG. 29J shows RNA-seq tracks for SP9 showing lower expression in HD-MSNs. FIG. 29K shows validation by qPCR with SP9-specific primers; Student's t-test; t=2.702 df=8; n=5 independent HD and control lines. FIG. 29L demonstrates that restoring SP9 expression by lentiviral transduction of SP9 at PID 14 rescues HD cell death phenotype at PID 35. Quantification of SYTOX-positive cells over Hoescht (F3,8=9.792, P=0.0047); n=averages of approximately 1,000 cells per group from 3 independent HD and control lines. FIG. 29A-FIG. 29F PID 30; FIG. 29I-FIG. 29L PID 35; FIG. 29J PID 32. FIG. 29A-FIG. 29L show a One-Way ANOVA with post hoc Tukey's test; **P<0.001; **P<0.01; *P<0.05; n.s.=not significant. Mean±s.e.m. Scale bar in FIG. 29A, FIG. 29E, 100 μm; in FIG. 29C, 10 μm; and in FIG. 29G, 500 μm.

FIG. 30A-FIG. 30B demonstrate spontaneous degeneration in culture is associated with loss of DARPP-32-positive neurons. Since major cell loss only occurs past PID 35, the levels of TUBB3-positive and DARPP-32-positive cells were quantified at PID 30 and PID 40 to determine extent to which DARPP-32-positive cells degenerate in culture. FIG. 30A shows Ctrl. 17c and HD.42 fibroblasts were transduced with miR-9/9*-124+CDM concurrently and immunostained at PID 30 and at PID 40. At PID 35 cells were confirmed to have altered levels of cell death (data are quantified and shown in the FIG. 35 of the main text). Non-transduced fibroblasts were used as a negative control for immunostaining. FIG. 30B shows from PID 30 to PID 40, there are no changes observed in the number of TUBB3-positive cells in control samples, in contrast to HD samples in which the level of TUBB3-positive cells is dramatically reduced (F3,8=32.25, P<0.0001). In addition, while the percent of DARPP-32-positive cells remains unchanged in control MSNs from PID 30 to PID 40, this percentage is reduced in HD samples at PID 40 in comparison to control (F3,8=5.211, P=0.0276). n=averages from 100 cells from 3 random fields-of-view from 3 biological replicates for each time point. One-Way ANOVA with post hoc Tukey's test; ***P<0.001; **P<0.01; *P<0.05; n.s.=not significant. Mean t s.e.m.

FIG. 31A-FIG. 31B show ATM inhibition by a small-molecule drug attenuates cell death levels and protects HD-MSNs against further oxidative insults. FIG. 31A demonstrates that treatment with KU60019 reduces cell death levels at PID 35; (F3,31=13.28, P<0.0001). FIG. 31B demontrates KU60019 protects HD-MSNs against H₂O₂-induced oxidative stress; (F3,31=12.58, P<0.0001). n=averages from 1,000 cells from 8 or 9 biological replicates; One-Way ANOVA with post hoc Tukey's test; ***P<0.001; **P<0.01; *P<0.05; n.s.=not significant. Mean±s.e.m. Scale bars: 100 μm

FIG. 32A-FIG. 32C are a series of images and graphs showing evidence of increased mitophagy in HD-MSNs. FIG. 32A shows that additional HD and control lines used in this study stain positive for TUBB3 and successfully undergo direct conversion by miR-9/9*-124+CDM. FIG. 32B shows LC3-II staining at multiple intervals during direct conversion. At PID 20, analysis of 3 independent HD and control lines shows that 2 out of 3 HD-MSNs lines have a higher number of autophagosomes, however the overall change was not significantly different. Students t-test; t=1.31 df=4 p=0.2604; n=averages from 10 cells per group. FIG. 32C show images and a bar graph of co-staining with MitoTracker Red and LC3-II at PID 40 in two pairs of HD and control lines showing a higher percentage of colocalization in HD-MSNs. (F3,8=61.48, P<0.0001); n=Averages from approximately 2,000 LC3-II puncta from 3 random field-of-views per group. Colocalization was measured using images acquired through confocal microscopy at 100× objective and analyzed post-acquisition by automated colocalization analysis. One-Way ANOVA with post hoc Tukey's test; ***P<0.001; **P<0.01; *P<0.05; n.s.=not significant. Mean±s.e.m.

FIG. 33A-FIG. 33D are a series of images and graphs showing mitochondrial and metabolic dysfunction in HD-MSNs. FIG. 33A shows MitoTracker Red staining showing that the total pool of mitochondria is unchanged between control and HD-MSNs; Students t-test; t=0.1034 df=4; n=averages of 100 cells from 3 independent HD and control lines. FIG. 33B show live imaging of active mitochondria by TMRE (tetramethylrhodamine, ethyl ester) revealing significant loss of mitochondrial membrane potential in HD-MSNs; Student's t-test; t=5.54 df=4; n=averages of 60 cells from 3 independent HD and control lines. FIG. 33C shows mitochondrial superoxide indicator, MitoSOX Red, showing increased superoxide production in HD-MSNs; Students t-test; t=9.384 df=4; n=averages of 100 cells from 3 independent HD and control lines. FIG. 33D shows accumulation of lipid droplets (LD) in HD-MSNs, visualized by Bodipy 493/503 dye; Students t-test; t=3.237 df=4; n=averages of 100 cells from 3 independent HD and control lines. Mean and S.E.M. ***P<0.001; **P<0.01: *P<0.05; n.s.=not significant. Scale bar FIG. 33A-FIG. 33C, 50 μm and FIG. 33D, 5 μm.

FIG. 34A-FIG. 34G are a series of illustrations, images, and graphs showing differential vulnerability to degeneration in distinct subtypes of HD neurons. FIG. 34A show HD.40 fibroblasts reprogrammed into cortical-like neurons (CNs) with miR-9/9*-124+DAM (NeuroD2, ASCL1 and Myt1L). FIG. 34B show HD.40 CNs immunostained with TUBB3 at PID 21. FIG. 34C show qPCR analysis for the expression of cortical genes as well as MSN-marker DARPP-32 at PID 30; n=3 samples per group; Student's t-test with Holm-Sidak correction, df=4. FIG. 34D shows a Comet assay; One-Way ANOVA (F3,195=20.15, P<0.0001) with post hoc Tukey's test; n=50 cells per group. FIG. 34E shows immunostaining and quantification of the DNA damage marker γH2AX; One-Way ANOVA (F3,8=18.23, P=0.0006) with post hoc Tukey's test; n=averages from 500 cells per group in 3 independent experiments. FIG. 34F shows SYTOX staining revealing differences in subtype-dependent cell death levels at PID 35; n=averages from 1,200 cells per group in 3 independent experiments; One-Way ANOVA (F3,8=29.2, P=0.0001) with post hoc Tukey's test. FIG. 34G shows HTT aggregation detected by MW8 antibody at PID 35 in HD-MSNs and HD-CNs and quantification of the percentage of cells with IBs in MSNs and CNs. n=averages from 100 cells per group in 3 independent experiments; One-Way ANOVA (F3,8=58.75, P<0.0001) with post hoc Tukey's test.***P<0.001; **P<0.01. Mean±s.e.m. Scale bars in FIG. 34B, FIG. 34D, and FIG. 34F are 100 μm; and in FIG. 34E, 20 μm.

FIG. 35A-FIG. 35D are a series of illustrations and images showing HD-MSNs reprogrammed from pre-symptomatic patients are less vulnerable to mHTT-induced toxicity. MSNs reprogrammed from 6 pre-symptomatic HD patients with 42-49 CAG repeats collected at least 13 years prior to disease onset are phenotypically normal despite bearing similar levels of mutant HTT inclusions as symptomatic HD-MSNs. FIG. 35A is a diagram depicting conversion of pre-clinical HD fibroblasts by miR-9/9*-124+CDM (Pre-HD MSNs). FIG. 35B shows all 6 primary fibroblasts samples from pre-clinical patients tested were successfully reprogrammed by miR-9/9*-124+CDM as shown by TUBB3 staining at PID 30. FIG. 35C-FIG. 35D are representative images and quantification of Ctrl-, Pre-HD and HD-MSNs at PID 35 assayed for cell death with SYTOX green (left column) (n=averages of 1,000 cells per group; F2,9=9.433, P=0.0062), oxidative DNA damage with 80H-dG (middle column) (n=averages of 100 cells per group; F2,9=21.8, P=0.0004), and mutant HTT inclusion bodies (IBs) (EM48) (right column) (n=averages of 100 cells per group; F2,9=9.911, P=0.0053). One-Way ANOVA with post hoc Tukey's test. ***P<0.001; **P<0.01; *P<0.05; n.s.=not significant.; Mean±s.e.m; n=averages from 3 independent control lines, 6 independent pre-HD lines and 3 independent HD lines. Scale bars=100 μm, except for HTT panel (left column) where scale bar=20 μm.

### DETAILED DESCRIPTION OF THE INVENTION

The present disclosure is based, at least in part, on the discovery that in addition to micro RNAs (e.g., miR-9/9*-124) transcription factors can be added to somatic cells (e.g., adult somatic cells, adult human fibroblasts, adult human fibroblast of mesodermal origin) to differentiate into clinically relevant neurons (e.g., motor neurons, MSNs). The present disclosure describes that motor neuron genes become accessible in response to miR-9/9*-124. More specifically, miR-9/9*-124 allows the subtype-specifying activities of ISL1 and LHX3. As described herein, chromatin profiling revealed a modular synergism between microRNAs and transcription factors allowing lineage-specific neuronal reprogramming, providing a platform for generating distinct subtypes of human neurons. More specifically, the present disclosure shows small non-coding RNAs, miR-9/9* and miR-124 (miR-9/9*-124), and motor neuron transcription factors ISL1 and LHX3, directly convert somatic cells, such as adult human fibroblasts into human motor neurons. The technology can be used for human motor neuron generation and a cellular platform for drug screening.

The present disclosure is also based at least in part on the discovery that Huntington's Disease (HD) patient fibroblasts can be converted to medium spiny neurons (MSNs) through microRNA-based neuronal conversion. One of the primary barriers in treating and studying devastating neurological diseases and traumas (e.g., ALS, SMA, spinal cord injury) is the inability to isolate and directly manipulate human motor neurons in the laboratory. The technology described herein enables the generation of motor neurons directly from patients, enabling disease modeling, and drug screening while simultaneously providing a source of patient specific cells for regenerative medicine.

As described herein, the disclosure provides for extensive characterization of the efficiency and specificity of fibroblast conversion. Briefly, through immunostaining analysis and gene expression profiling it has been demonstrated that converted motor neurons (Moto-miNs) phenotypically resemble endogenous motor neurons. It was also determined that Moto-miNs behave as motor neurons through functional testing through electrophysiological and co-culture tests. Furthermore, the expression of hallmark motor neuron genes in Moto-miNs derived from multiple donor ages was directly compared to the human spinal cord and verified similar expression levels.

As described herein, a method of using small non-coding RNAs, miR-9/9* and miR-124 (miR-9/9*-124), and motor neuron transcription factors ISL1 and LHX3 have been discovered and optimized to directly convert adult human fibroblasts into human motor neurons. This technology enables the direct study and of the cell type affected in diseases (e.g., Amyotrophic Lateral Sclerosis (ALS) and Spinal Muscular Atrophy (SMA)) using patient-specific motor neurons. Unique to this method is its utility of combining microRNAs and transcription factors for optimal efficiency, specificity, speed, ease of use, and importantly, the retention of both donor age marks and positional information. These features are currently believed to be important components in designing a cellular platform for drug screening.

The present disclosure contributes to the fields of developmental biology, regenerative medicine, direct conversion, neuroscience, genetics, chromatin biology, and microRNA biology.

Recent studies on cell-fate reprogramming have demonstrated the capability of generating post-mitotic neurons by directly converting an unrelated cell type such as fibroblasts. While direct cell-fate conversion presents great potential in disease-modeling and regenerative medicine, its promise strictly depends on the feasibility of converting primary human somatic cells from adults —the ideal source of cells for patient-specific disease modeling and regenerative therapy. Most studies on neuronal reprogramming focus on the utility of transcription factors using mouse fibroblasts. Unfortunately, the transcription factor-based conversion approaches that work in mouse cells often fail to efficiently generate functionally mature neurons when tested in human adult fibroblasts. This difficulty is reflected in the current hypothesis that fully differentiated human somatic cells contain epigenetic barriers that need to be overcome to allow the transition of cell-fates.

It has been demonstrated that ectopically expressing small non-coding microRNAs (miRNAs), miR-9/9′, and miR-124 (miR-9/9*-124), with transcription factors in human adult fibroblasts is sufficient to generate functionally mature neuronal subtypes (Yoo et al., Nature, 2011; Victor et al., Neuron, 2014). Interestingly, the same transcription factors in the absence of miR-9/9*-124 do not display reprogramming activities, suggesting miRNAs are capable of opening the neurogenic potential of human fibroblasts. However, the molecular events underlying the miRNA-induced resolution of the cell-fate barrier remain poorly understood.

As described herein, a series of experiments were carried out employing genome-wide DNA methylation analysis, chromatin accessibility analysis by ATAC-seq, genome-wide transciptome analyses, and cellular/electrophysiological analyses of human adult fibroblasts expressing miR-9/9*-124 to reveal the surprising potency of these miRNAs alone in inducing epigenetic and cellular remodeling leading to the adoption of a neuronal ground state. It was further demonstrated how this miRNA-induced neuronal state can be specified into a highly pure population of human spinal cord motor neurons by expression of transcription factors, ISL1 and LHX3, with miR-9/9*-124. The results are summarized briefly in the points below.

As described herein, it was discovered that expression of miR-9/9*-124 alone without any transcription factors in human adult fibroblasts is sufficient to generate a neuronal fate characterized by mature functionality and activation of a pan-neuronal genetic program. MiR-9/9*-124 also evoked extensive changes in the expression of multiple genes encoding regulators of chromatin, such as DNA-methylation-modifying proteins, proteins involved in histone modifications, and components of chromatin remodeling complexes.

Surprisingly, miR-9/9* and miR-124 led to extensive epigenetic remodeling characterized by active reconfiguration of differentially methylated regions in the genome and changes (opening and closing) in chromatin accessibilities. The miRNA-induced epigenetic state that was detected is neuronally primed in that genes involved in neurogenesis and neuronal function activate, while genes associated with a fibroblast fate are repressed. Importantly, it was found that miR-9/9*-124 induced opening of neuronal gene loci embedded in the heterochromatic regions present in human fibroblasts. This is in contrast to “pioneer” transcription factors that are capable of binding closed loci, but are unable to open large regions of the genome, further demonstrating the potency of these miRNAs as neurogenic effectors.

During miRNA-induced neuronal conversion, genomic loci associated with pan-neuronal genes open up and are expressed, whereas loci for subtype-associated genes are induced to open but are not activated. These results presented molecular insight into how the miRNA-induced neuronal state is unspecified yet poised to receive inputs from subtype-lineage determinants (often referred to as terminal selectors).

Further building on these findings, transcription factors expressed in motor neurons were screened to identify transcription factors that would synergize with miR-9/9*-124 to specifically generate human motor neurons, the cell type affected in devastating neurological diseases such as amyotrophic lateral sclerosis (ALS), spinal muscular atrophy (SMA) and spinal cord injury. By transcriptome analyses, motor neuron-specific programs activated by ISL1 and LHX3 in the background of miR-9/9*-124 were also identified.

Electrophysiology analyses revealed that 45/45 motor neurons patched, from young and old donors, had the ability to fire action potentials demonstrating the impressive functional maturity of converted motor neurons. To further validate the authenticity of motor neuron conversion, translating ribosome affinity purification (TRAP)-seq on in vivo mouse motor neurons were performed and directly compared their gene expression to converted motor neurons. Collectively, the results showed an unprecedented level of subtype specificity and functional maturity of human spinal cord motor neurons generated by the direct conversion of human adult fibroblasts.

Altogether, here is presented important epigenetic insights into how the miRNA-based reprogramming modality may serve as a platform for generating multiple clinically relevant neuronal subtypes. The present findings also provide further insights into previously demonstrated subtype-specific conversion (Yoo et al., Nature, 2011; Victor et al., Neuron 2014). Understanding such control for subtype-specificity is important particularly for modeling late onset neurological diseases that selectively affect distinct subtypes of neurons, as it was recently found that miRNA-based neuronal conversion retains the cellular age stored in the original human fibroblasts (Huh et al., eLife, 2016). This is in contrast to the resetting of age to an embryonic stage seen in induced pluripotent stem cells.

Mechanistic insights into the biological phenomena of transdifferentiation have broad implications in diverse fields of biology. First, the observation that miRNAs alone are sufficient to elicit cell-fate conversion phenotypically demonstrates that small non-coding RNAs can function as potent cell-fate regulators, much more than fine-tuning gene expressions. Indeed, the present study will provide novel insights into understanding how small RNA molecules regulate chromatin states during cellular differentiation or reprogramming. Second, due to the conversion efficiency, specificity, speed, and functional maturity seen in converted motor neurons, many researchers will employ the neuronal conversion technique presented in this study to derive spinal cord motor neurons from patients with inherited motor neuron diseases towards the goal of modeling the disease using patient neurons. Lastly, the epigenome and transcriptome datasets, provided herein, describing the epigenetic changes in the miRNA-induced neuronal state, provides a platform to derive additional neuronal subtypes directly from adult human fibroblasts. Indeed, the motivation to devise a reprogramming approach to generate human motor neurons started with the discovery that motor neuron genes become accessible in response to miR-9/9*-124, which has been successfully achieved as shown in the present disclosure.

Advances in the understanding of genetic pathways that specify neuronal cell fates during development have has enabled the directed differentiation of pluripotent stem cells into specific neuronal subtypes. This knowledge has been further leveraged to directly convert (reprogram) non-neuronal somatic cells into neurons. These direct conversion modalities can be valuable in the study of late-onset neurodegenerative diseases, as the original age of human fibroblasts is maintained in converted neurons in contrast to the cellular rejuvenation observed in induced pluripotent stem cells (Horvath, 2013; Miller et al., 2013). However, little is known about the epigenetic and molecular events that accompany direct cell-fate conversion limiting the utility of these features.

The present disclosure provides for cell-conversion agents. Cell conversion agents can convert cells (e.g., somatic cells) and convert them into neurons (e.g., MSNs). Cell conversion agents can comprise small non-coding RNA (micro RNA) and transcription factors.

Small Non-Coding RNA (Micro RNA)

As described herein, neuronal microRNAs, such as miR-9/9* and miR-124 (miR-9/9*-124) to direct cell-fate conversion of adult human fibroblasts to post-mitotic neurons and, with additional transcription factors, enable the generation of discrete neuronal subtypes. Previously, the molecular events underlying the neurogenic switch mediated by microRNAs during neuronal reprogramming were unknown. Here, the neurogenic state induced by miR-9/9*-124 expression alone was systematically dissected alone and reveal the surprising capability of miR-9/9*-124 in coordinately stimulating the reconfiguration of chromatin accessibility, DNA methylation and mRNA levels, leading to the generation of functionally excitable miRNA-induced neurons, yet uncommitted towards a particular subtype-lineage.

miRNA (e.g., miR-9/9*)

As described herein, it was discovered that micro RNAs (miRNAs) (e.g., miR-9/9*-124) concertedly and separately target components of genetic pathways that antagonize neurogenesis and promote neuronal differentiation during neural development.

The miRNAs capable of converting neurons are capable of converting somatic cells into neurons can coordinate epigenetic and transcriptional changes resulting in neuronal cell fate conversion; induce a generic neuronal state characterized by the loss of fibroblast identity, the presence of a pan-neuronal gene expression program, and absence of subtype specificity; initiate subunit switching within BAF chromatin remodeling complexes while separately repressing the neuronal cell-fate inhibitors REST, Co-REST, and SCP1; or alter the expression of genes involved in DNA methylation, histone modifications, chromatin remodeling, and chromatin compaction.

The miRNAs as described herein can concertedly and separately target components of genetic pathways that antagonize neurogenesis and promote neuronal differentiation during neural development; open the neurogenic potential of adult human fibroblasts and thus provides a platform for subtype-specific neuronal conversion of human cells; orchestrate widespread neuronal chromatin reconfiguration; or promote the opening of neuronal subtype-specific loci, but are not expressed.

A microRNA is a small non-coding RNA molecule (e.g., containing about 22 nucleotides) that can be found in plants, animals, and some viruses, that can function in RNA silencing and post-transcriptional regulation of gene expression. While the majority of miRNAs are located within the cell, some miRNAs, commonly known as circulating miRNAs or extracellular miRNAs, have also been found in an extracellular environment, including various biological fluids and cell culture media.

miRNAs can be encoded by eukaryotic nuclear DNA in plants and animals and by viral DNA in certain viruses whose genome is based on DNA. miRNAs function via base-pairing with complementary sequences within mRNA molecules. As a result, these mRNA molecules are silenced, by one or more of the following processes: cleavage of the mRNA strand into two pieces, destabilization of the mRNA through shortening of its poly(A) tail, or less efficient translation of the mRNA into proteins by ribosomes.

miRNAs can resemble the small interfering RNAs (siRNAs) of the RNA interference (RNAi) pathway, but miRNAs derive from regions of RNA transcripts that fold back on themselves to form short hairpins, whereas siRNAs can derive from longer regions of double-stranded RNA. The human genome can encode over 1000 miRNAs, which are abundant in many mammalian cell types and appear to target about 60% of the genes of humans and other mammals.

MicroRNAs (miRNAs) can regulate genetic pathways by binding to their target transcripts and repressing their expression. Target specificity can be governed largely through short sequence complementarity within the 5′ end of a miRNA enabling a single miRNA to target hundreds of mRNA transcripts. Moreover, a single mRNA can be targeted by multiple miRNAs, markedly enlarging the effect on single gene repression (Wu et al., 2010). These attributes position miRNAs to affect broad changes in gene expression and genetic programs despite their limited size. The convergence of genetic controls by miRNAs towards a specific biological process is exemplified by miR-9/9*- and miRNA-124 miRNAs activated at the onset of neurogenesis. For example, miR-9* and miR-124 can synergistically act as a molecular switch to initiate subunit switching within BAF chromatin remodeling complexes while separately repressing the neuronal cell-fate inhibitors REST, Co-REST, and SCP1. These examples suggest that miR-9/9* and miR-124 target components of genetic pathways that antagonize neurogenesis to promote a neuronal identity during development.

It has been shown that co-expressing the neuronal miRNAs, miR-9/9*-124, with TFs enriched in the cortex and striatum is sufficient to directly convert primary adult human fibroblasts to cortical and striatal medium spiny neurons, respectively. Furthermore, the expression of region-specific TFs alone or substituting miR-9/9*-124 with ASCL1 is insufficient to convert human fibroblasts. Therefore, the use of subtype-specifying TF activity to confer terminal identity during miRNA-mediated neuronal conversion may be reminiscent to in vivo terminal selector TFs which, upon determination of a neuronal fate, initiate and advance mature subtype-identities. However, the existence and utility of such a neuronal state during microRNA-induced neuronal reprogramming has yet to be determined.

Transcription Factors

In addition to showing the surprising capability of miRNAs, such as miR-9/9′-124, in coordinately stimulating the reconfiguration of chromatin accessibilities, it has been further shown that the microRNA-induced neuronal state enables additional transcription factors, such as ISL and LHX3, to selectively commit conversion to a highly homogenous population of human spinal cord motor neurons (Moto-miNs). Furthermore, it has been shown striatal-enriched factors (e.g., CTIP2, DLX1, DLX2 and MYT1L (CDM)) with miR-9/9-124 generate MSNs from adult human fibroblasts. Taken together, the disclosure reveals a modular synergism between microRNAs and transcription factors that allows lineage-specific neuronal reprogramming, providing a platform for generating distinct subtypes of human neurons.

The transcription factors as described herein can be administered in any method known in the art. For example transcription factors can be provided exogenously or expressed ectopically.

Striatal-Enriched Transcription Factors

As described herein, striatal-enriched factors (e.g., CTIP2, DLX1, DLX2 and MYT1L (CDM)) with miR-9/9*-124 have been shown to generate MSNs from adult human fibroblasts, yielding a neuronal population comprised of about 70-80% of MSNs.

Motor Neuron Transcription Factors

As described herein, motor neuron factors, ISL1 and LHX3, can function as terminal selectors to specify neuronal conversion to a highly enriched population of human spinal cord motor neurons. Plasticity of the miRNA-induced state was further demonstrated by directly converting adult human fibroblasts into a highly pure population of motor neurons through the addition of motor neuron enriched TFs, ISL, and LHX3, thereby presenting a modular method to directly convert human fibroblasts into desired neuronal subtypes.

Neurogenic Transcription Factors

As described herein, neurogenic transcription factors, CTIP2, DLX1, DLX2, and MYT1L (CDM) can reprogram fibroblasts into cortical-like neurons (CN).

Converted Neurons and Disease Models Derived from Patient Fibroblast Cells

The present disclosure provides for converted neurons and uses thereof for models of disease using a patients fibroblast cells, ectopic expression of microRNAs, and transcription factors (see e.g., Example 2).

As described herein, the identification of miRNA-induced neurogenic state has provided molecular insights into how multiple neuronal subtypes can be generated from patient fibroblasts for modeling neurological diseases. For example, the methods described herein can be used to convert a somatic cell such as a fibroblast cell (e.g., human fibroblast cells) into a converted neuron. The somatic cell or fibroblast cell can be any somatic cell or fibroblast capable of being converted using any of the methods as described herein. The converted neuron can be a microRNA-induced neuron (miN). For example, the converted neuron can be a motor neuron, a spinal motor neuron, a cortical neuron, a cortical-like neuron, a striatal medium spiny neuron (MSN), a dopaminergic neuron, a GABAergic neuron, a cholinergic neuron, serotonergic neuron, or a glutamatergic neuron.

Ectopic expression of brain-enriched microRNAs (miRNAs), such as miR-9/9′ and miR-124 (miR-9/9*-124), in human adult fibroblasts have been shown to directly convert fibroblasts to neurons. The miR-9/9-124-mediated conversion, partially afforded by their activity in controlling chromatin remodeling complexes, can be guided to specific and mature neuronal subtypes with the co-expression of transcription factors. As such, striatal factors (or striatal-enriched factors) CTIP2, DLX1, DLX2, and MYT1L (CDM) with miR-9/9-124 have been shown to generate MSNs from adult human fibroblasts, yielding a neuronal population comprised of 70-80% of MSNs. Given that iPSC-based protocols have reported MSN conversion efficiencies of only 5%-10% and more recently 20-40%, the MSN-specific neuronal conversion that generates a neuronal population highly enriched with MSNs from HD patients will offer a useful tool to model HD. Moreover, in contrast to neurons differentiated from iPSCs in which the age stored in original fibroblasts is erased during the induction of pluripotency^{20,21}, directly converted neurons has been shown to retain age-associated marks of starting adult human fibroblasts, including the epigenetic age (also known as the epigenetic clock), oxidative stress, DNA damage, miRNAome, telomere lengths and transcriptome^{22,23}. This unique feature offers potential advantages in modeling adult-onset disorders using directly converted neurons, yet the value of MSNs converted from HD patients' fibroblasts in disease modeling has not been determined.

The generation of HD patient-derived MSNs (HD-MSNs) through miR-9/9′-124-CDM-based conversion of fibroblasts is reported herein. The present disclosure focused on HD samples with CAG repeat ranges in the 40s, which represent the majority of HD cases, in contrast to previously reported studies on modeling HD with CAG repeat numbers longer than 60 CAG repeats. It was found that HD-MSNs captured many HD-associated phenotypes, including formation of aberrant protein aggregates, mHTT-induced DNA damage, spontaneous degeneration over time in culture, and decline in mitochondrial function. Furthermore, by inducing HD fibroblasts into iPSCs and redifferentiating them back into fibroblasts for miRNA-based neuronal conversion, it was discovered that differences in mHTT aggregation propensity observed in these two distinct cellular reprogramming methods are the result of drastically different levels of proteasome activity. Intriguingly, modifying the terminal neuronal cell fate to cortical neurons in directly reprogrammed HD cells alleviated mHTT-induced toxicity through reduced DNA damage and reduced cell death. Furthermore, MSNs reprogrammed from six pre-symptomatic HD patients, sampled at least 13 years before the clinical onset of the disease, were less vulnerable to mHTT-induced toxicity despite the marked presence of mHTT aggregates. These data highlight the advantages of direct neuronal conversion offers for modeling age-related phenotypes of late-onset diseases with enriched populations of specific neuronal subtypes. While the applicability of iPSCs for the development of stem cell-based therapies and modeling of developmental processes remains unequivocal, the present findings address many of the challenges for modeling adult-onset HD.

In Huntington's disease (HD), expansion of CAG codons within the Huntington gene (HTT) leads to the aberrant formation of protein aggregates and the differential degeneration of striatal medium spiny neurons (MSNs). Modeling HD using patient-specific MSNs has been challenging, as neurons differentiated from induced pluripotent stem cells are free of aggregates and lack an overt cell death phenotype. Here MSNs from HD patient fibroblasts were generated through microRNA-based neuronal conversion, which has previously been shown to bypass the induction of pluripotency and retain age signatures of original fibroblasts. It was found that patient MSNs consistently exhibited mutant HTT (mHTT) aggregates, and spontaneous degeneration overtime in culture that was preceded by mHTT-dependent DNA damage. Further evidence is provided that erasure of age stored in starting fibroblasts or diverting conversion into cortical neurons resulted in differential manifestation of cellular phenotypes associated with HD, highlighting the importance of age and neuronal subtype specificity in modeling late-onset neurological disorders.

Huntington's disease (HD) is a progressive neurodegenerative disorder caused by the abnormal expansion of CAG codons within the first exon of the Huntington (HTT) gene^{1,2}. HD symptoms typically manifest in midlife, and include motor deficits, psychiatric symptoms and cognitive decline³. While healthy individuals have an average HTT CAG tract size of 17-20 repeats, HD patients have an expansion of 36 or more CAGs⁴. Moreover, CAG repeat length is directly correlated to severty of the disease and inversely related to age of onset, with abnormally large CAG expansions (>60 repeats) leading to juvenile onset^{5,6}. Expanded CAG trinucleotides encode a polyglutamine stretch (PolyQ) that can accumulate into proteinaceous cytoplasmic and intranuclear aggregates that are generally thought to be neurotoxic⁷, although the formation of inclusion bodies has also been suggested as a neuroprotective mechanism⁸.

A striking characterstic of HD pathology is the selective degeneration of stratal medium spiny neurons (MSNs), while other neuronal subpopulations are relatively spared. Due to the clinical importance of MSNs and the lack of treatment capable of halting HD onset and progression, many protocols have been developed to generate MSNs from induced pluripotent stem cells (iPSCs) to establish patient-specific platforms for disease modeling¹⁰⁻¹².

Studies modeling HD with patient-specific iPSC-derived MSNs have, however, only uncovered mild phenotypes, often requiring additional cellular insults^{10,11,13-15}. For example, striatal neurons generated through HD-iPSCs demonstrated elevated levels of caspase activity only upon trophic factor withdrawal, treatment with hydrogen peroxide or high levels of glutamate, but otherwise displayed no overt cell death phenotype^{11,13,16}. In addition, neurons differentiated from iPSCs of HD patients did not display mutant HTT (mHTT) aggregates even after the addition of cellular stressors¹⁰, and in other studies required culturing for at least 6-8 months and treatment with proteasome inhibitors before aggregates were detected^{14,15}. Therefore, a different reprogramming approach that generates a homogenous population of patient-derived MSNs that display HD phenotypes more robustly will offer an alternative cellular platform for disease modeling and drug screening.

Neurodegeneratve Diseases, Disorders, and Conditions

The present disclosure provides for methods and compositions for treating or modeling neurodegenerative (e.g., neurological, motor neuron) diseases, disorders, or conditions or screening for therapeutics for neurological diseases, disorders, or conditions. For example, a neurodegenerative disease, disorder, or condition can be Abulia; Agraphia; Alcoholism; Alexia; Alien hand syndrome; Allan-Hemdon-Dudley syndrome; Alternating hemiplegia of childhood; Alzheimer's Disease (AD); Amaurosis fugax; Amnesia; Amyotrophic lateral sclerosis (ALS); Aneurysm; Angelman syndrome; Anosognosia; Aphasia; Apraxia; Arachnoiditis; Amold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder Bell's palsy; Brachial plexus injury; Brain damage; Brain injury; Brain tumor, Brody myopathy: Canavan disease; Capgras delusion; Carpal tunnel syndrome; Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation: Chorea; Chronic fatigue syndrome; Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia: Corticobasal degeneration; Cranial arteritis; Craniosynostosis; Creutzfeldt-Jakob disease; Cumulative trauma disorders; Cushing's syndrome; Cyclothymic disorder Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis: Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting: Familial spastic paralysis; Febrile seizures: Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis: Giant cell inclusion disease; Globoid Cell Leukodystrophy; Gray matter heterotopia; Guillain-Barre syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster; Hirayama syndrome; Hirschsprung's disease; Holmes-Adie syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Isodicentric 15; Joubert syndrome: Karak syndrome: Kearns-Sayre syndrome; Kinsboume syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease (See amyotrophic lateral sclerosis); Lumbar disc disease; Lumbar spinal stenosis; Lyme disease-Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy: Morvan syndrome; Motor Neurone Disease—see amyotrophic lateral sclerosis; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis: Myelinoclastic diffuse sclerosis; Myodonic Encephalopathy of infants; Myoclonus; Myopathy; Myotubular myopathy; Myotonia congenita; Narcolepsy; Neuro-Behget's disease; Neurofibromatosis; Neuroleptic malignant syndrome: Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal leaming disorder; O'Sullivan-McLeod syndrome; Occipital Neuralgia; Occult Spinal Dysraphism Sequence; Ohtahara syndrome; Olivopontocerebellar atrophy; Opsoclonus myoclonus syndrome; Optic neuritis; Orthostatic Hypotension; Otosclerosis: Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy: Ramsay Hunt syndrome type I; Ramsay Hunt syndrome type II; Ramsay Hunt syndrome type III—see Ramsay-Hunt syndrome; Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder Romberg syndrome; Saint Vitus dance; Sandhoff disease; Schilder's disease (two distinct conditions); Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury (SCI); Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome.

Motor Neuron Disease

As described herein, the present disclosure provides for the treatment of a neurodegenerative disease (e.g., a motor neuron disease) using a converted neuron, by expression of miR-9/9* and miR-124 (miR-9/9-124) and transcription factors (i.e., ISL1 and LHX3) in a human adult fibroblast.

In some embodiments, the neurodegenerative disease, disorder or condition can be a motor neuron disease (MND). Motor neuron diseases (MNDs) are a group of progressive neurological disorders that can destroy motor neurons, the cells that control essential voluntary muscle activity such as speaking, walking, breathing, and swallowing. A motor neuron disease can be an inherited disease with symptoms including difficulty or inability to grip, walk, speak, swallow, or breathe; a weakened grip, which can cause difficulty picking up or holding objects; weakness at the shoulder that makes lifting the arm difficult: a “foot drop” caused by weak ankle muscles; dragging of the leg; or slurred speech (dysarthra).

As another example, a motor neuron disease can be Amyotrophic Lateral Sclerosis (ALS), Spinal Muscular Atrophy (SMA), or Spinal Cord Injury (SCI). Other motor neuron diseases can include frontotemporal dementia, progressive bulbar palsy, pseudobulbar palsy, primary lateral sclerosis (PLS), progressive muscular atrophy, Spinal muscular atrophy (SMA) (e.g., SMA type 1, also called Werdnig-Hoffmann disease; SMA type II; congenital SMA with arthrogryposis; Kennedy's disease, also known as progressive spinobulbar muscular atrophy), or post-polio syndrome (PPS).

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transduction”, as used herein, is a process by which foreign DNA is introduced into a cell (e.g., by a virus, viral vector, bacteriophage, naked DNA). Transduction methods are well known; see e.g., Transduction, Genetic at the US National Library of Medicine Medical Subject Headings (MeSH). Except as otherwise noted herein, therefore, the process of the present disclosure can be carried out in accordance with such processes. For example, as described herein, miRNAs and transcription factors can be cloned into a viral vector (e.g., a lentivirus plasmid, Sendai virus). For example, after cloning into the viral vector, a virus (e.g., lentivirus) is produced, and the fibroblasts are infected. The virus then integrates its genome (containing the miRNAs and TFs) into the fibroblast genome. As such, these ectopic genes are stably expressed by the transduced cells. A viral vector can be any viral vector known in the art. For example, the viral vector can be a retrovirus, a lentivirus, an adenovirus, or an adeno-associated virus.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cel nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95/6-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by lle, Leu by lle, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of downregulation or silencing genes are known in the art. For example, expressed protein activity can be downregulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyrbonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoom and Lieberman (2005) Annual Review of Medicine 56, 401-423, describing RNAi). RNAi molecules are commercially available from a variety of sources (e.g., Ambion, Tex.; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a neurodegenerative disease, disorder, or condition in a subject in need administration of a therapeutically effective amount of converted neurons, so as to substantially inhibit a neurodegenerative disease, disorder, or condition, slow the progress of a neurodegenerative disease, disorder, or condition, or limit the development of a neurodegenerative disease, disorder, or condition.

Methods described herein are generally performed on a subject in need thereof. A subject in need of the therapeutic methods described herein can be a subject having, diagnosed with, suspected of having, or at risk for developing a neurodegenerative disease, disorder, or condition. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of converted neurons is, for example, that amount that would cause the desired therapeutic effect in a subject while minimizing undesired side effects. In various embodiments, an effective amount of converted neurons described herein can substantially inhibit a neurodegenerative disease, disorder, or condition, slow the progress of a neurodegenerative disease, disorder, or condition, or limit the development of a neurodegenerative disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of converted neurons can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit a neurodegenerative disease, disorder, or condition, slow the progress of a neurodegenerative disease, disorder, or condition, or limit the development of a neurodegenerative disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of converted neurons can occur as a single event or over a time course of treatment. For example, converted neurons can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a neurodegenerative disease, disorder, or condition.

Converted neurons can be administered simultaneously or sequentially with another agent, such as an antibiotic, an anti-inflammatory, or another agent. For example, converted neurons can be administered simultaneously with another agent, such as an antibiotic or an anti-inflammatory. Simultaneous administration can occur through administration of separate compositions, each containing one or more of converted neurons, an antibiotic, an anti-inflammatory, or another agent. Simultaneous administration can occur through administration of one composition containing two or more of converted neurons, an antibiotic, an anti-inflammatory, or another agent. Converted neurons can be administered sequentially with an antibiotic, an anti-inflammatory, or another agent. For example, converted neurons can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening for SP9 modulating agent or a candidate drug or therapeutic agent (e.g., a pharmacological factor, a genetic factor). An SP9 modulating agent can be any agent that can modulate SP9 expression.

For example, a method of screening compositions for an SP9 modulating agent can comprise obtaining cells from a subject; contacting the cells with a suspected SP9 modulating agent; and measuring the expression of SP9 on the cells.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45,177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363: Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Microrna-Induced Direct Conversion of Human Froblasts into Motor Neurons (Moto-Mins)

The following example describes microRNA-induced epigenetic remodeling during direct cell-fate conversion of adult human fibroblasts. The following example shows (1) microRNAs open the neurogenic potential in human adult fibroblasts; (2) miR-9/9* and miR-124 orchestrate widespread neuronal chromatin reconfiguration; (3) neuronal subtype-specific loci open in response to microRNAs but are not expressed; and (4) terminal selector genes guide this modular neuronal state to human motor neurons.

As evidenced in this example, miR-9/9*-124 concertedly and separately targets components of genetic pathways that antagonize neurogenesis and promote neuronal differentiation during neural development.

Advances in the understanding of genetic pathways that specify neuronal cell fates during development have enabled the directed differentiation of embryonic and induced pluripotent stem cells (iPSCs) into specific neuronal subtypes. This knowledge has been further leveraged to directly convert (or reprogram) non-neuronal somatic cells into neurons via ectopic expression of pro-neural transcription factors (TFs) or neurogenic miRNAs with TFs, bypassing the induction of pluripotency. Specifically, ectopically expressing small non-coding microRNAs (miRNAs), miR-9/9* and miR-124 (miR-9/9*-124), with transcription factors in human adult fibroblasts is sufficient to generate functionally mature neuronal subtypes. These direct conversion modalities may prove invaluable in the study of late-onset neurodegenerative diseases, as the original age of human fibroblasts is maintained in converted neurons in contrast to the cellular rejuvenation observed in iPSCs. Interestingly, the miRNA-mediated reprogramming approach boasts high conversion efficiency in adult human fibroblasts, which may provide unique opportunities in modeling neurological disorders using patient derived neurons (Victor et al., 2014). However, despite the advantages of direct reprogramming, little is known about the epigenetic and molecular events that accompany direct cell-fate conversion. Here, mechanistic insights into the cell-fate pioneering activity of miR-9/9*-124 are provided. The present results demonstrate that miRNAs induce remodeling of chromatin accessibilities, DNA methylation and the transcriptome leading to the generation of functionally excitable neurons. Surprisingly, during neuronal reprogramming, miR-9/9*-124 opens neuronal gene loci embedded in heterochromatic regions while simultaneously repressing fibroblast loci, revealing how miRNAs may overcome the cell-fate barrier that exists in human fibroblasts. These findings led to the discovery of a miRNA-induced permissive neurogenic ground state capable of generating multiple, clinically relevant neuronal subtypes. As such, it has been shown that the addition of motor neuron factors, ISL1 and LHX3, can function as terminal selectors to specify neuronal conversion to a highly enriched population of human spinal cord motor neurons. Altogether, these studies identify miRNA-mediated epigenetic remodeling events underlying direct neuronal conversion of human fibroblasts.

Co-expressing miR-9/9* and miR-124 (miR-9/9*-124), with TFs enriched in the cortex and striatum directly converts primary adult human fibroblasts to cortical and striatal medium spiny neurons, respectively (Victor et al., 2014; Yoo et al., 2011). However, the same TFs without miR-9/9*-124 fail to trigger neuronal conversion (Victor et al., 2014: Yoo et al., 2011), suggesting that the miRNA-induced neuronal state is permissive to terminal selector TFs which, upon determination of a neuronal fate, initiate and advance mature subtype-identities.

As described herein, the miRNA-induced neuronal state in adult human cells was identified and systematically investigated.

Longitudinal analyses of the transcriptome, genome-wide DNA-methylation and chromatin accessibilities revealed that miR-9/9*-124 induced extensive remodeling of the epigenome, including simultaneous activation of a pan-neuronal program and the reconfiguration of chromatin accessibilities. These changes precede the emergence of differentially methylated genomic regions. Because miR-9/9*-124 also led to the opening of genomic loci for multiple subtype-specific genes including established motor neuron markers, it was postulated that motor neuron-enriched transcription factors would cooperate with miR-9/9*-124 to specify a motor neuron lineage. As such, it was demonstrated that co-expressing Ts ISL1 and LHX3 along with miR-9/9*-124 generate a highly pure population of human spinal cord motor neurons. Taken together, these results demonstrate that miR-9/9*-124 opens the neurogenic potential of adult human fibroblasts and provides a platform for subtype-specific neuronal conversion of human cells.

(A) Neuronal Conversion of Human Adult Fibroblasts with miR-9/9*-124 Alone

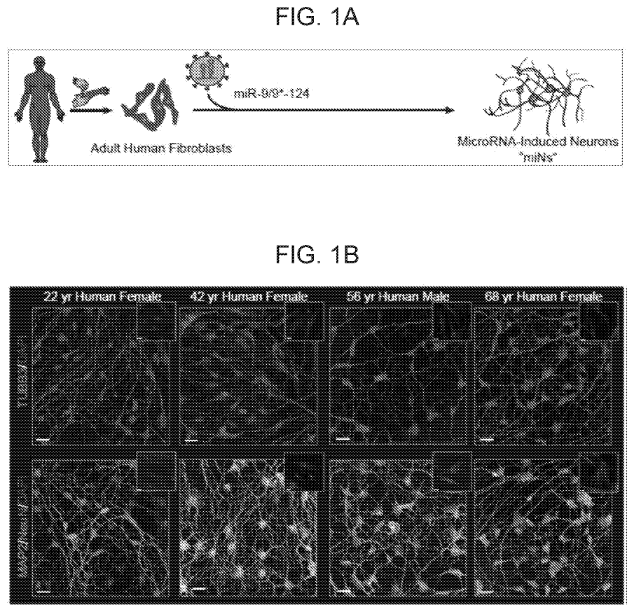

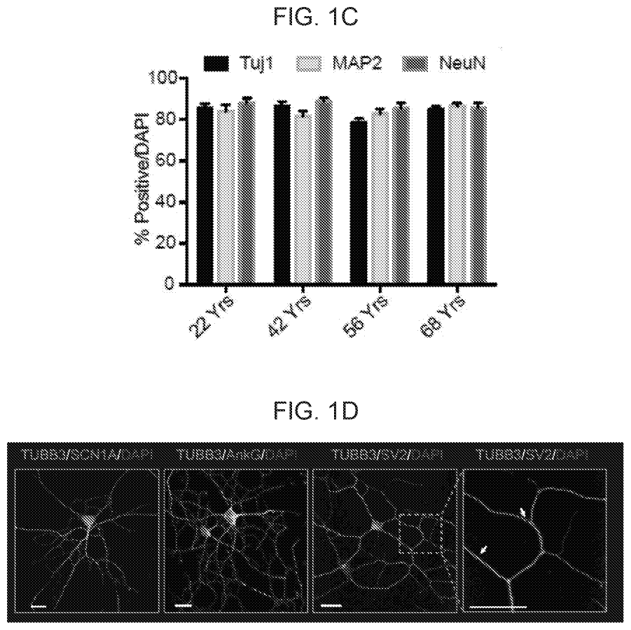

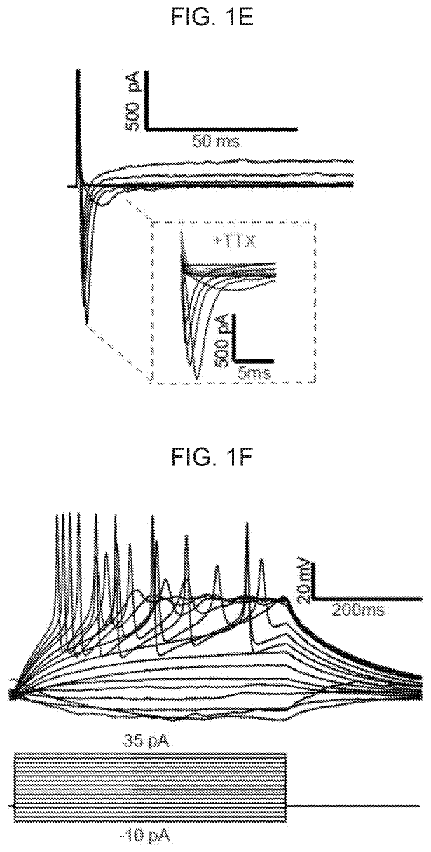

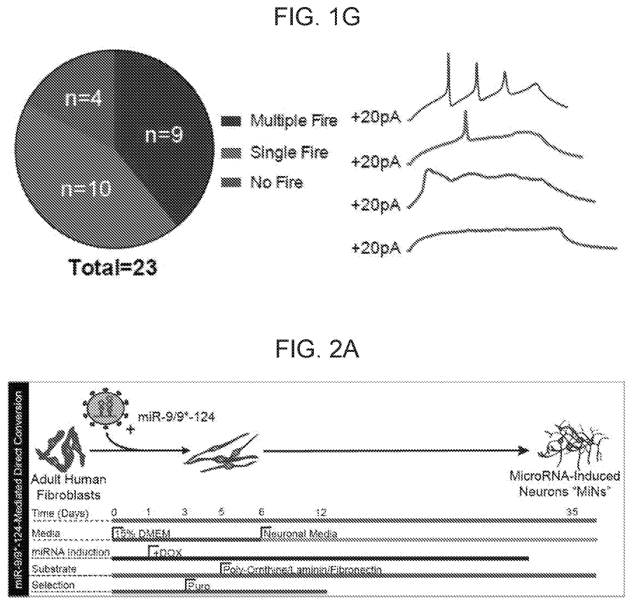

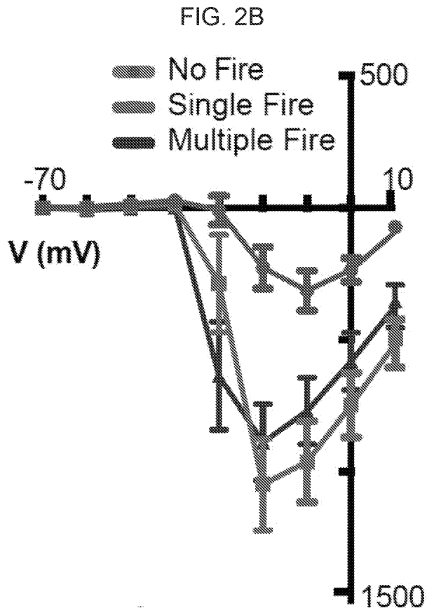

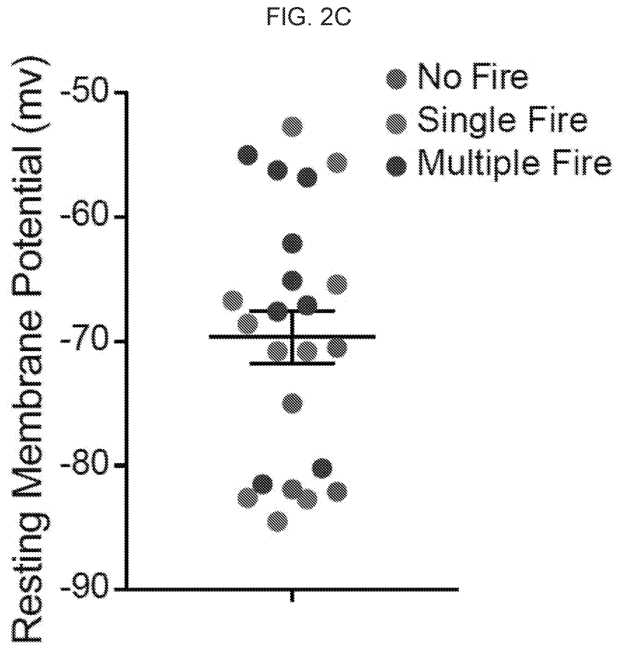

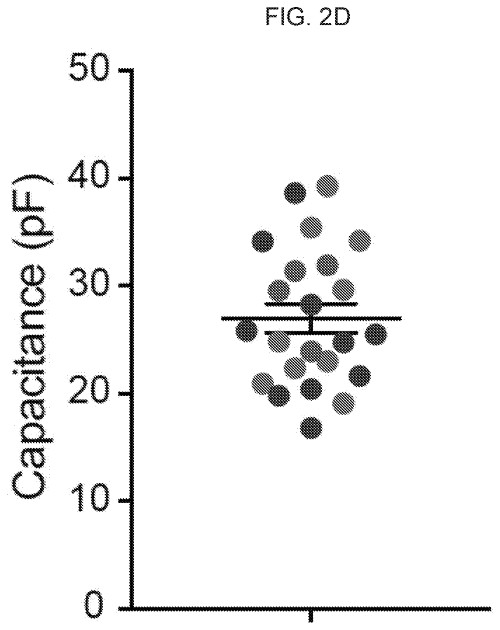

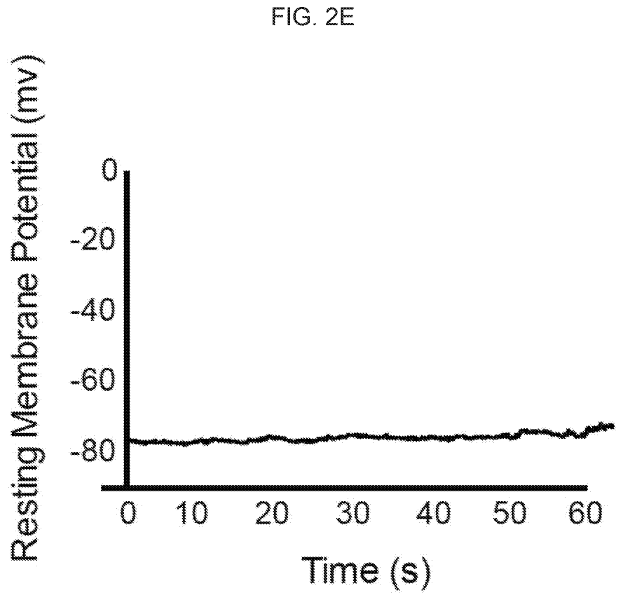

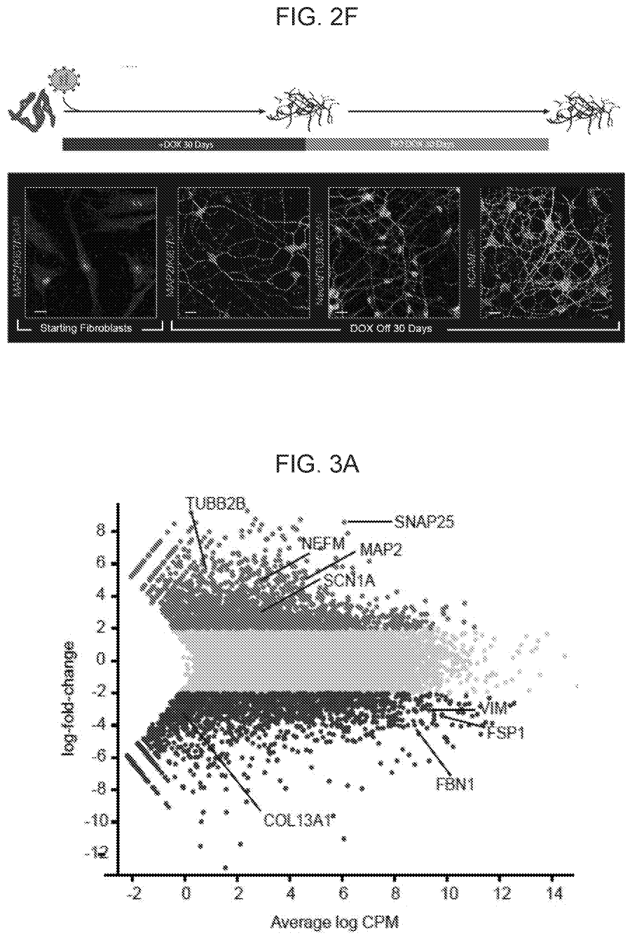

To dissect how miRNAs alone contribute to neuronal conversion, the ability of miR-9/9*-124 was first tested to convert primary human fibroblasts collected from multiple adult individuals from ages 22 to 68 into microRNAs-induced neurons (miNs). The multiple fibroblast samples were transduced with lentivirus containing a doxycycline-(Dox-) inducible promoter driving miR-9/9*-124 and BCL-XL (Victor et al., 2014) (see e.g., FIG. 1A and FIG. 2A). At 35 days post-transduction, the cel morphology was evaluated by examining the expression of neuronal markers MAP2, TUBB3, and NEUN by immunohistochemistry (see e.g., FIG. 1B). Strikingly, miR-9/9*-124 alone, converted 80% of the fibroblasts to neuronal cells displaying complex neurite outgrowth, and neuronal marker expression (see e.g., FIG. 1B and FIG. 1C). The converted cells stained positive for voltage-gated sodium channels SCN1A and Ankyrin G, which localized at axonal initial segments with a characteristic polarized staining pattern (see e.g., FIG. 1D). The synaptic vesicle marker SV2 displayed defined puncta along neurites, consistent with the adoption of a neuronal fate (see e.g., FIG. 1D).

(B) Functional Properties and Stability of MiRNA-Induced Neurons

To determine if miNs displayed membrane excitability, whole-cell recording on miNs in a mono-culture condition was performed without co-culturing with glial or primary neurons. 100% of the cells recorded (23 out of 23 cells) exhibited fast TTX-sensitive inward currents upon depolarization (see e.g., FIG. 1E), while 19/23 cells fired action potentials (Aps) during current injections(9/23 cells fired multiple APs, 10/23 fired single APs) (see e.g., FIG. 1F and FIG. 1G). Similar current (I)-voltage (V) curve relationships were observed between cells that fired multiple or single action potentials (see e.g., FIG. 2B). All miNs had a stable hyperpolarized resting membrane potential ranging from −52.7 to −84.5 mV with a mean value of −69.63 mV±2.15 mV (S.E.M.) (see e.g., FIG. 2C and FIG. 2D). A correlation between capacitance and firing patterns were not observed suggesting this membrane property would not serve as an accurate measure of neuronal maturation during direct conversion (see e.g., FIG. 2E). Together, this data indicates that miNs exhibit the membrane functionality of neurons.

The minimum duration of miRNA expression required for neuronal conversion was determined by inactivating the doxycycline-inducible promoter at 3-day intervals by Dox removal beginning at day 9 until reprogramming day 30 (FIG. 2F). Loss of fibroblast identity and gain of neuronal identity was assayed by analyzing fibroblast-specific protein (FSP1) and MAP2 expression, respectively. Surprisingly, there was a reduction in the number of FSP1-positive cells and the appearance of MAP2-positive cells after only 9 days of miRNA expression, however; efficient switching of a cell population from FSP1- to MAP2+ required 30 days ofmiR-9/9*-124 (see e.g., FIG. 2F). These data reveal the non-synchronous process of the neuronal conversion and temporal requirements for highly efficient neuronal conversion. The stability of miRNA-induced neuronal conversion was determined by following miNs for an additional 30 days after removing miR-9/9*-124 exposure (see e.g., FIG. 2G, top). The majority of miNs remained as post-mitotic cells (marked by the absence of Ki67, a cell proliferation marker) expressing MAP2, TUBB3, NeuN, and NCAM, in contrast to non-converted fibroblasts (see e.g., FIG. 2G, left panel), indicating that the morphological and protein expression changes that accompany miR-9/9*-124-mediated conversion of adult human fibroblasts are stable after 30 days of miR-9/9*-124 expression.

(C) Transcriptional Profiling of miNs

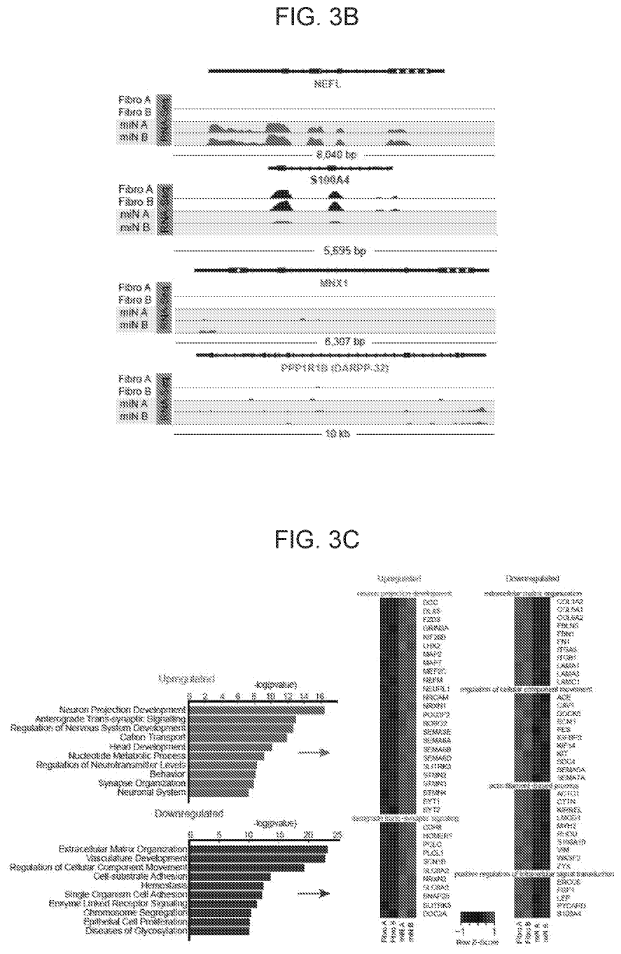

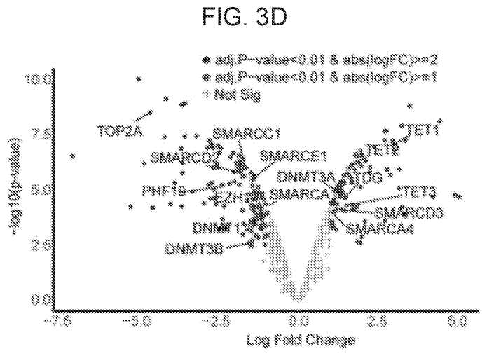

To further explore the miR-9/9*-124-mediated neuronal output, the transcriptome of starting human adult fibroblasts and miNs were profiled after 30 days of neuronal conversion by RNA-Seq. 2,692 differentially expressed genes (DEGs) were identified in miNs representing 1,251 up-regulated and 1,441 downregulated genes in comparison to fibroblasts (log fold change 22; adj.P-value <0.01) (see e.g., FIG. 3A). A robust downregulation of fibroblast-specific genes (for instance, S100A4, VIM, FBN1 and, COL13A1) was accompanied by an enrichment of pan-neuronal genes including, MAP2, SCN1A, SNAP25, NRCAM, and NEFM (see e.g., FIG. 3A and FIG. 3B top two traces).

Analysis of top 10 gene ontology (GO) terms revealed that upregulated genes in miNs are primarily enriched with terms related to neuronal development and functionality (see e.g., FIG. 3C) while downregulated genes in associate with fibroblast functions (see e.g., FIG. 3C). Downregulated genes also included key cell-cycle components (data not shown), consistent with the previous finding that miR-9/9*-124 expression in human fibroblasts caused rapid cell cycle exit without transitioning through a neural stem cell-like state (Yoo et al., 2011). Interestingly, neuronal subtype-specific genes such as TH (dopaminergic neurons), GABBR2, GABR1, and GAD2 (GABAergic neurons), CHAT (cholinergic neurons), or DARPP-32 (striatal medium spiny neurons), were not significantly enriched in miNs (see e.g., FIG. 3B, bottom two traces as examples). Overall, the transcriptome analyses show miR-9/9*-124 induce a neuronal state characterized by the loss of fibroblast identity and the presence of a pan-neuronal gene expression program without a commitment to a particular subtype.

(D) transcriptional Changes in Epigenetic Machinery

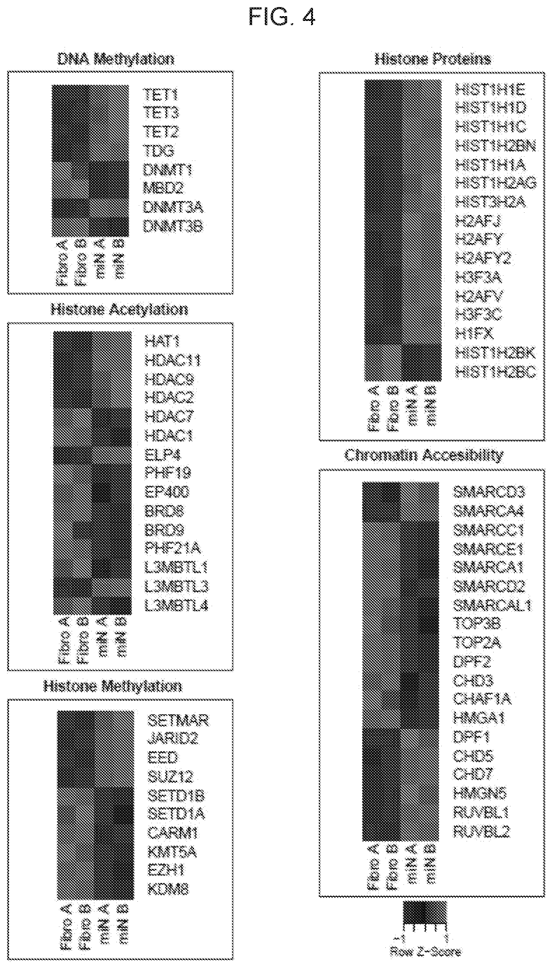

Epigenetic modifications can markedly affect gene expression and developmental programs (Cantone and Fisher, 2013). The presently disclosed gene expression studies showed that when compared to fibroblasts, miNs had markedly altered expression of genes involved in DNA methylation, histone modifications, chromatin remodeling, and chromatin compaction (see e.g., FIG. 3D and FIG. 4). For instance, the TET family of proteins, key mediators of DNA-demethylation (Wu and Zhang, 2011) were upregulated along with the brain-enriched de novo DNA-methyltransferase DNTM3A (Lister et al., 2013), while DNMT3B (Okano et al., 1999) mRNA levels were reduced in miNs compared to fibroblasts (see e.g., FIG. 3D and FIG. 4). Transcripts encoding histones and histone variants were altered (see e.g., FIG. 4) suggesting that changes in histone composition may accompany neuronal conversion of human fibroblasts. Genes encoding chromatin remodelers important for neurogenesis like CHD5, CHD7 and components of the BAF chromatin remodeling complex were expressed at higher levels in miNs than in fibroblasts (Egan et al., 2013: Feng et al., 2013; Lessard et al., 2007) (see e.g., FIG. 3D and FIG. 4). Additionally, the main DNA topoisomerase 2 family member expressed in miNs is TOP2B, which replaces the non-neuronal TOP2A, a switch that has been observed during normal neuronal differentiation (Tiwari et al., 2012) (see e.g., FIG. 3D, FIG. 4). In sum, dynamic changes and switches within diverse epigenetic modifiers coincide with neuronal differentiation and appear to be recapitulated in direct neuronal conversion of fibroblasts by miR-9/9*-124.

(E) Dynamic Regulatory Events During Neuronal Reprogramming

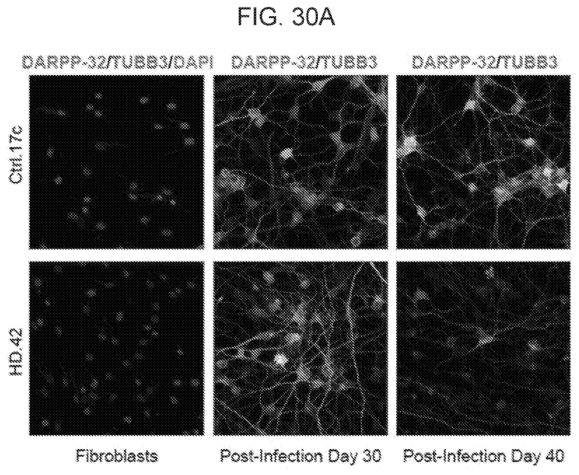

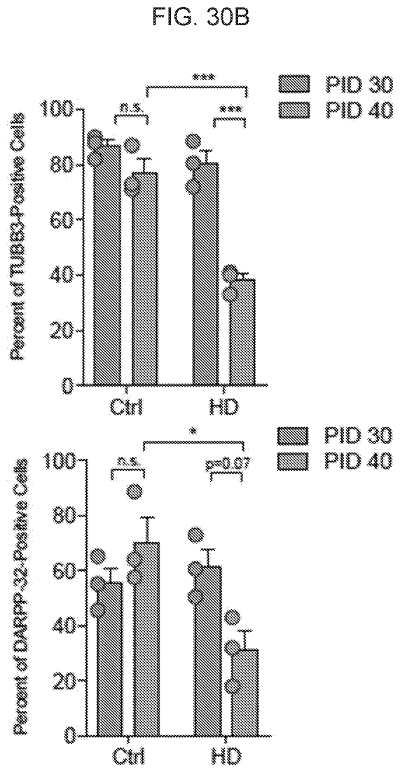

Because transcriptome profiling at day 30 only provided a snapshot of the functional output of neuronal reprogramming, transcriptome dynamics were explored by profiling intermediary timepoints (days 3, 6, 10, and 20) by RNA-seq. The Dynamic Regulatory Events Miner (DREM) (Schulz et al., 2012) reports 13 paths of co-regulated, differentially expressed genes during the first 20 days of neuronal conversion (FIG. 30A). Combining DREM with predicted TF-gene binding interactions (Ernst et al., 2010) revealed several potential TFs associated with major regulatory events (bifurcations in each path; FIG. 30A). Altogether, major regulatory events were observed before day 10, suggesting genetic networks are established within 10 days of miR-9/9-124 expression. Thereafter, the directionality of gene expression stays the same but transcript levels markedly change. This transcriptional maturation over time may explain why the acquisition of functional neuronal characteristics requires 30 days of culture. GO analyses of each path revealed enrichment of neuronal terms in the most upregulated path, whereas downregulated paths were enriched for cell cycle and extracellular matrix-related terms (FIG. 30B), consistent with results for day 30 transcriptome profiling (FIG. 3C). The transcriptome switch from a fibroblast to a neuronal program was seen as early as day 10 of miR-9/9*-124 expression (FIG. 30C), while genes associated with synaptic functionality (e.g. HOOK1) are activated at a later time point (day 20, FIG. 30C).

Finally, there were no significant changes in ASCL1 or SOX2, TFs that have been used to reprogram somatic cells into neurons (Niu et al., 2013; Pang et al., 2011), suggesting miR-9/9*-124-induced neuronal conversion activates a neuronal program through mechanisms distinct from those previously reported.

(F) DNA Methylation Profiling of miNs

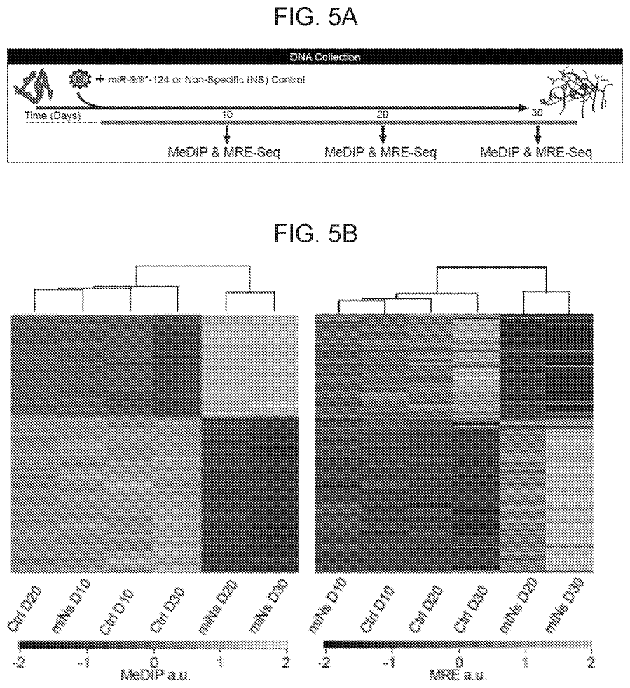

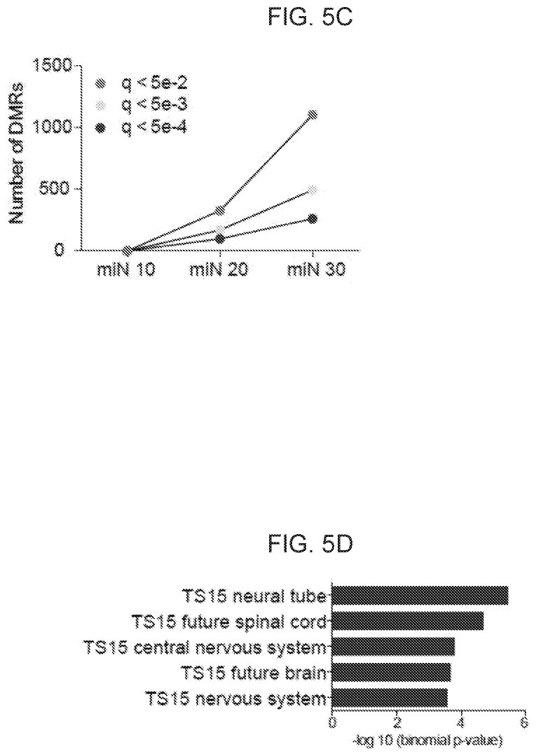

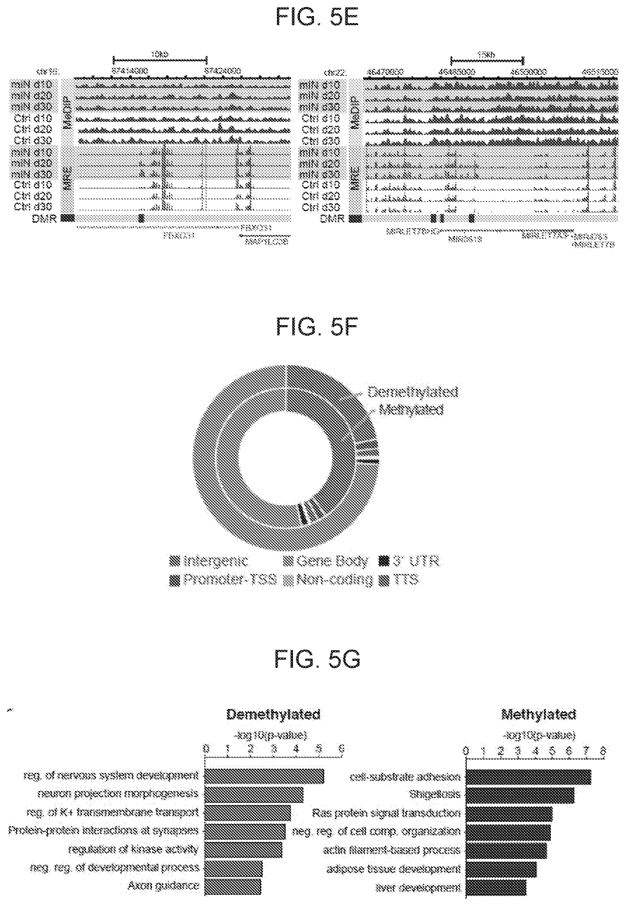

After observing numerous changes in DNA methylation machinery, genome-wide DNA methylation was assessed at an early (day 10), intermediate (day 20), and late stage (day 30) of neuronal reprogramming fibroblasts by combining methylated DNA immunoprecipitation sequencing (MeDIP-seq) and methylation sensitive restriction enzyme sequencing (MRE-seq; FIG. 5A) (Zhang et al., 2013). No significant changes in DNA methylation were detected at day 10 between cells that were exposed to miRNAs and non-treated control (ctrl) cells. In contrast, 1,540 differentially methylated regions (DMRs) were identified at day 20 (miN day 20 vs. Ctrl day 20) that overlap with DMRs at day 30 (miN day 30 vs. Ctrl day 30; FIG. 5B and FIG. 5C). The difference in DNA methylation at day 30 between treated and control cells was more dramatic than changes observed at day 20 with most DNA regions (Stadler et al., 2011; Xie et al., 2013; Zhang et al., 2013). Gene ontology (GO) analysis of Mouse Genome Informatics (MGI) expression using the GREAT (McLean et al., 2010) tool to characterize the top overlapping DMRs, in miNs enriched only for neuronal tissue developmental processes (see e.g., FIG. 5D). Interestingly, these differentially methylated regions undergo changes at TS15 in mouse development (equivalent to mouse E9/10), which is when miR-9 expression is first detected in developing mouse telencephalon (Shibata et al., 2008). Two examples of top DMRs within genes important for neuronal development and function (Gehrke et al., 2010; Vadhvani et al., 2013) are shown in FIG. 5E. Nearly 64% of DMRs were found to be located in introns and 28% of DMRs are in intergenic regions (see e.g., FIG. 5F). Comparison data from day 30 of demethylated and methylated DMRs present and RNA-seq revealed 882 differentially expressed genes in total. GO analysis of top demethylated DMRs associated with upregulated genes (>2.5 logFC) was enriched for neuronal terms. In contrast, top DMRs associated with downregulated genes (<2.5 logFC) did not match GO terms involved in neuronal development (see e.g., FIG. 5G; data not shown). Collectively, these data indicate that miRNA-mediated DMRs occur primarily within genes involved in neuronal development during neuronal reprogramming. Furthermore, since miR-9/9*-124 expression quickly induces cell cycle exit these changes in DNA methylation must necessarily occur via active processes, such as those catalyzed by TET and TDG enzymes (Kohli and Zhang, 2013), rather than via failure to remethylate DNA after DNA replication.

(G) Chromatin Remodeling in miNs

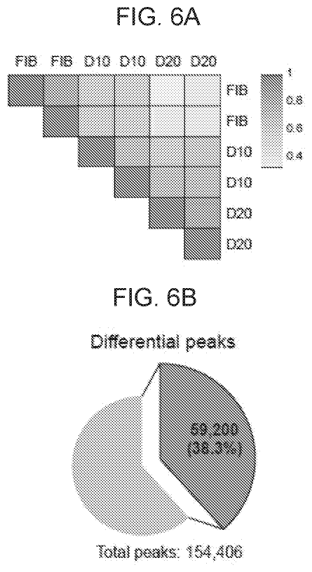

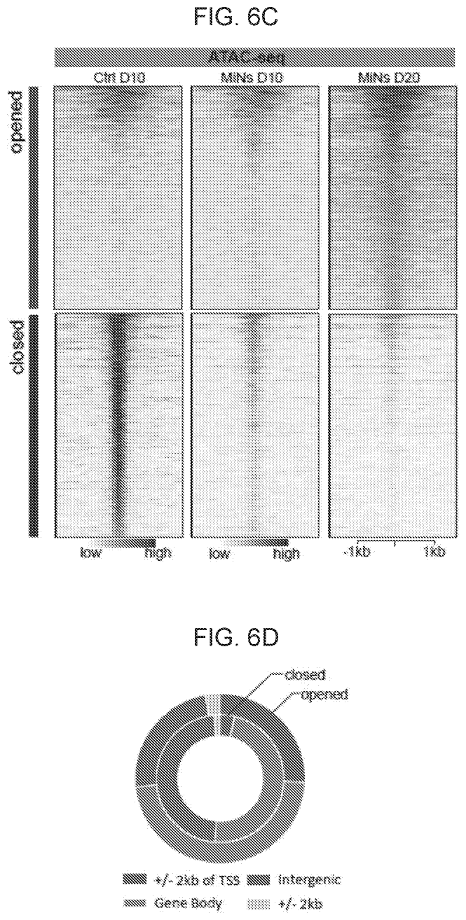

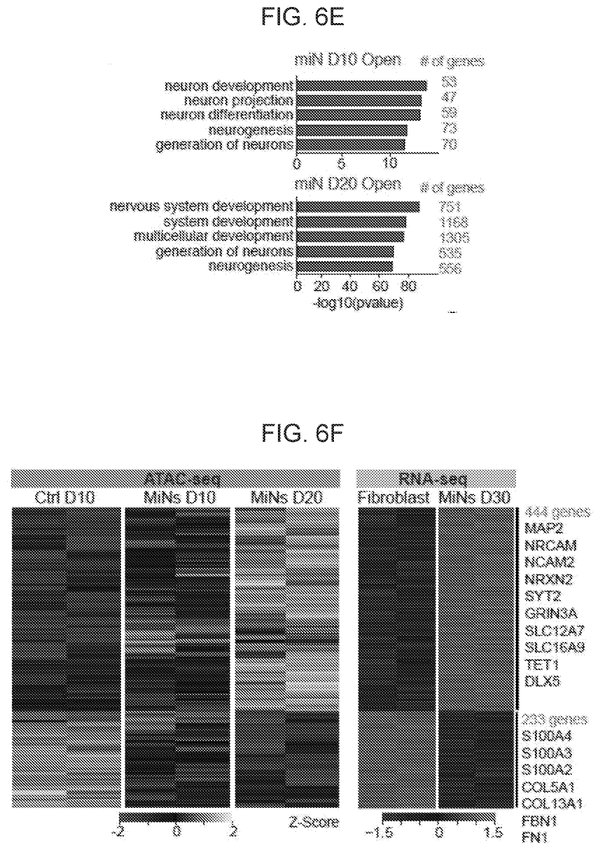

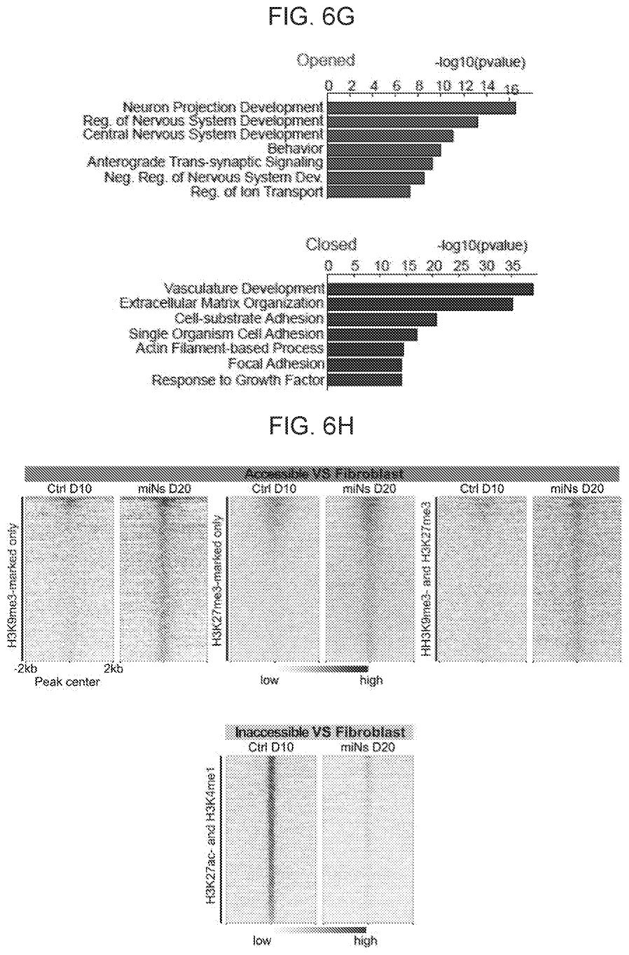

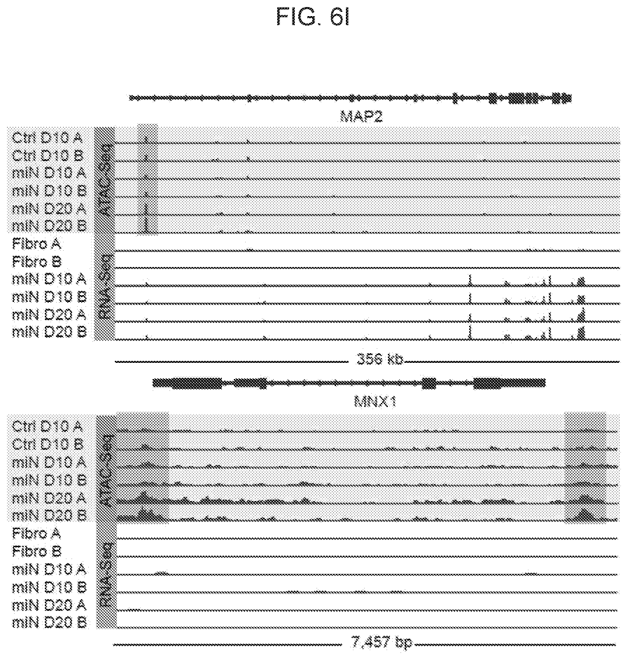

Extensive expression changes in diverse chromatin remodeling genes during neuronal reprogramming (see e.g., FIG. 3D and FIG. 4) suggests that miR-9/9*-124 may also alter chromatin accessibility. Thus an Assay for Transpose-Accessible Chromatin was performed followed by high throughput sequencing (ATAC-seq) 10 and 20 days after initiating miR-9/9*-124 induced neuronal conversion. A high correlation between replicates confirmed the reproducibility of the presently disclosed analyses (see e.g., FIG. 6A). 154,406 total peaks were obtained across all samples and 59,200 differential peaks were identified (see e.g., FIG. 6B). Of the total peaks detected, 20,712 became more accessible (open) and 38,882 peaks became inaccessible (closed) were identified. Most of differential peaks in MiNs at day 10 overlapped with the peaks at day 20 and the signal intensity of peaks gradually increased or decreased during the conversion (see e.g., FIG. 6C) suggesting gradual transition of chromatin accessibility during reprogramming (see e.g., FIG. 6C). Interestingly, it was discovered that the ratio of open intragenic to distal intergenic regions increased during miR-9/9*-124-mediated conversion (see e.g., FIG. 4D).

(H) Erasure of Fibroblast Epigenetic Identity and Gain of Neuronal Chromatin Architecture

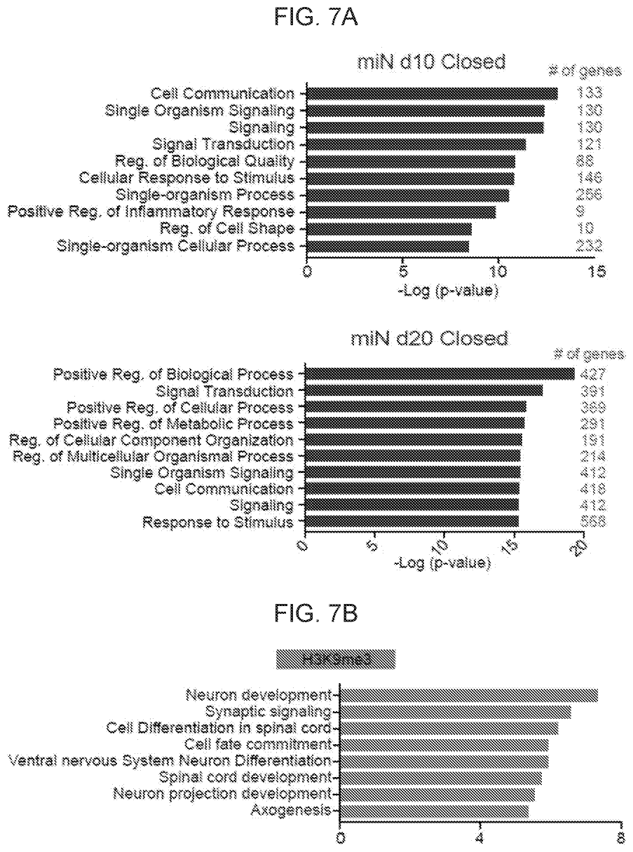

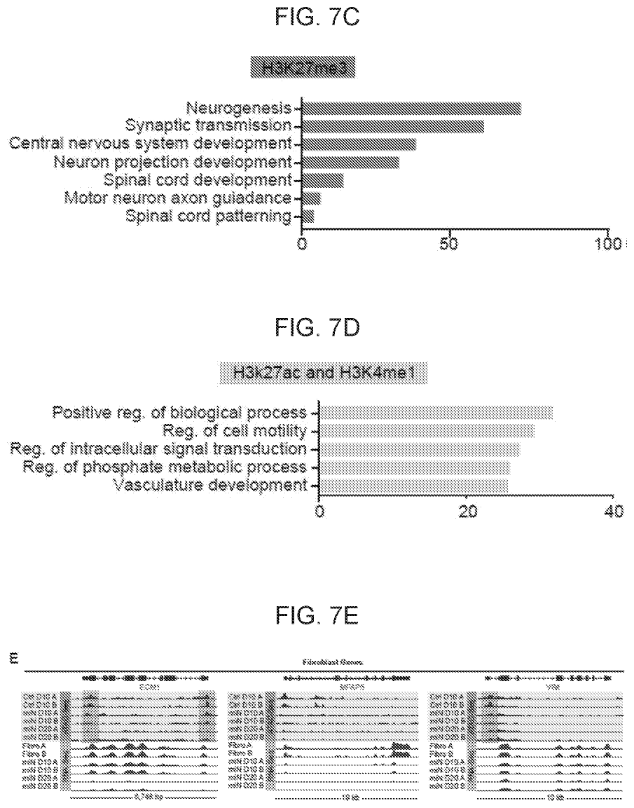

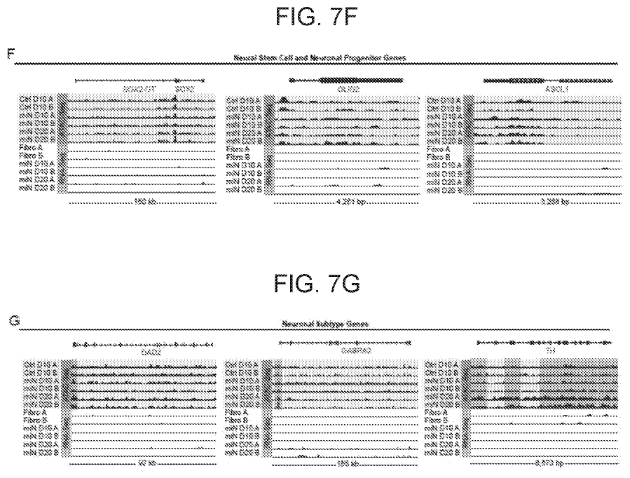

To gain insight into biological relevance of changes in chromatin accessibility after miR-9/9*-124 expression, gene enrichment analysis was performed on genes with differential ATAC signals around the transcription start site ±2 Kb (TSS). 4,915 genes were identified with gradual increases and 1,763 genes were identified with gradual decreases in ATAC signals during conversion. Top GO terms associated with genes with increased ATAC signals are enriched in neuronal terms (including nervous system development, generation of neurons and neurogenesis) (see e.g., FIG. 6 E), while GO terms associated genes with closed regions were not (see e.g., FIG. 7A). It was found that the number of genes in open regions associated with neuronal terms gradually increased from day 10 to 20, consistent with the transcriptional profiling and coinciding with bona fide neuronal commitment of miNs (see e.g., FIG. 6E). Consistent with this hypothesis, the chromatin accessibility for fibroblast marker genes like S100A4, S100A10, VIM and COL13A1, was found to gradually decrease between 10 and 20 days into neuronal conversion (see e.g., FIG. 7A). Collectively, the ATAC-seq analyses demonstrate miR-9/9*-124-induced chromatin remodeling events are characterized by concurrent closing of fibroblast-related genomic loci and opening of neuronal gene loci.

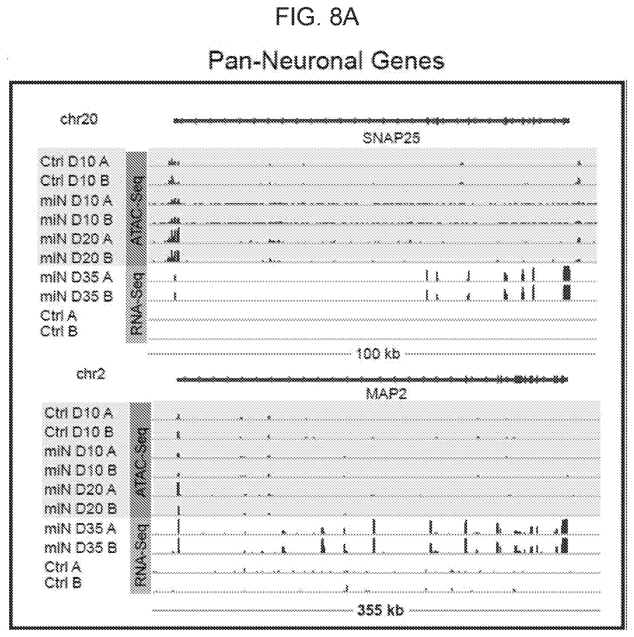

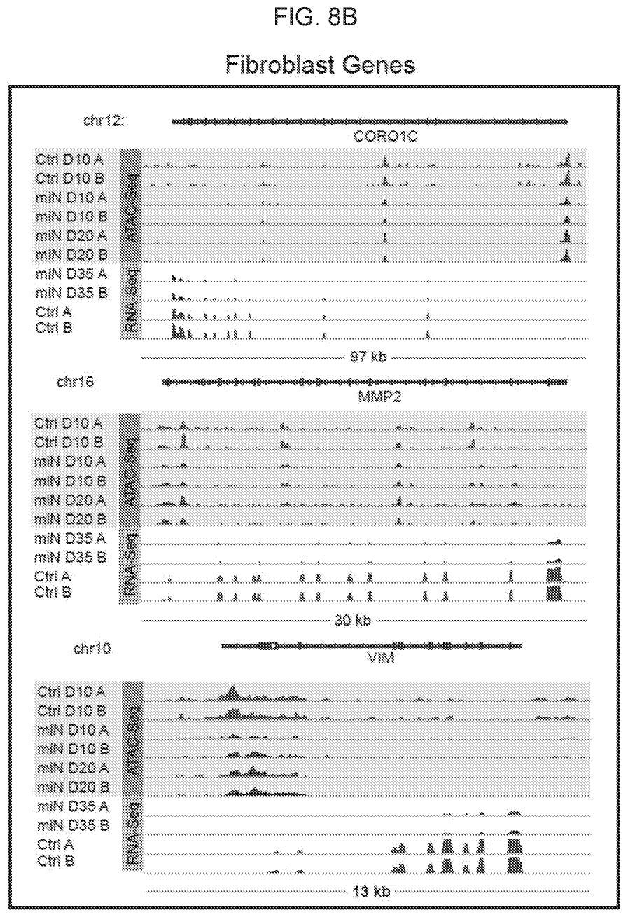

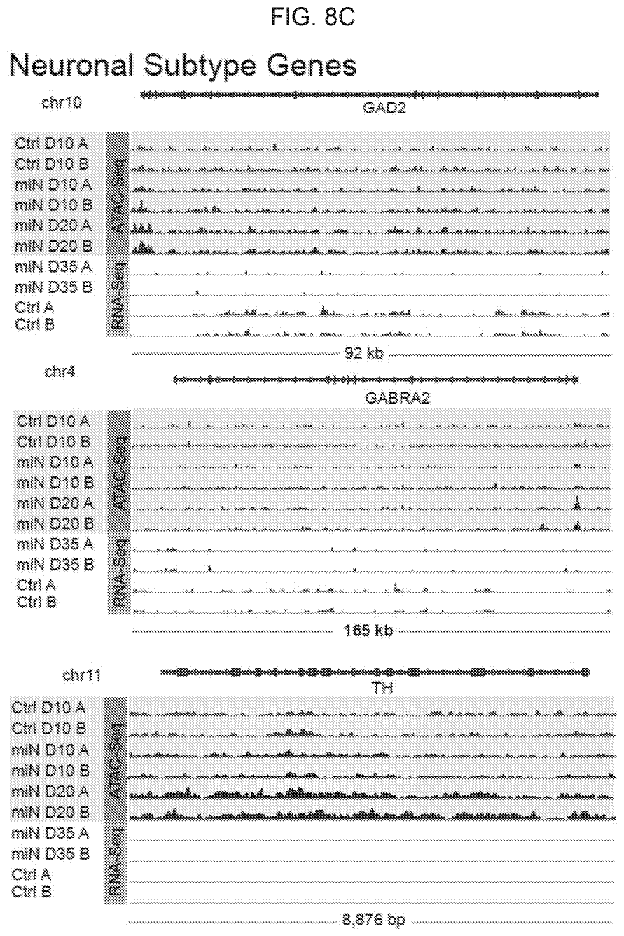

Next, whether miR-9/9*-124-induced chromatin accessibility is correlated with changes in mRNA levels was examined. DEGs (logFC>2 or <−2 adjusted P-value <0.01) were compared to genes with altered chromatin accessibility around the TSS and identified 501 upregulated and 184 downregulated genes that coincide with open and closed regions, respectively (see e.g., FIG. 6F). GO enrichment analysis revealed that upregulated genes from open regions associate with neurogenic terms, while downregulated genes from closed regions are connected with terms important for fibroblast function (see e.g., FIG. 6G, see FIG. 8A and FIG. 8B for example tracks). This demonstrates the concordant regulation of transcription and chromatin accessibility in during reprogramming. Interestingly, some opened genomic regions that displayed no changes in gene expression contained genes that are uniquely expressed in neuronal subtypes, including those enriched in: dopaminergic neuron markers (TH and SLC6A3), serotonergic neuron markers (FEV, LMX1B and SLC6A4), GABAergic neuron markers (SLC6A1, SLC32A1, GAD2), striatal medium spiny neuron marker (PPP1R1B), glutamatergic neuron markers (GLUL and SLCA6), and cholinergic or motor neuron markers (MNX1, CHAT, and SLC5A7) (see e.g., FIG. 8C for example tracks). These results suggest that miR-9/9*-124 poise chromatin to accept additional inputs from subtype-specifying determinants, without activating subtype-specific programs. In all, the chromatin dynamics observed during miRNA mediated neuronal conversion are consistent with time-dependent suppression of fibroblast identity concurrent with the opening of neuronal loci and activation of pan-neuronal gene expression.

(I) MicroRNA-induced Chromatin Remodelinq at Heterochromatin Regions in Fibroblasts

To gain a more complete understanding of the epigenetic architecture within opened and closed chromatin sites the relationship of these regions to pre-existing histone marks present in fibroblasts was examined. It was hypothesized that regions that close during reprogramming would overlap with the active enhancer/euchromatin marks, H3K27ac and H3K4me1 in fibroblasts. Conversely, regions that open during neuronal reprogramming may overlap with heterochromatic H3K9me3 and H3K27me3 signatures pre-existing in fibroblasts.

70,661 regions commonly marked by H3K27ac and H3K4me1 and 5,843 regions commonly marked by H3K9me3 and H3K27me3 in human fibroblasts were selected based on the Roadmap Epigenome database (Roadmap Epigenomics et al., 2015). The regions with altered chromatin accessibility overlapped with these histone marks. Strikingly, it was found that 1,128 ATAC signal peaks present in day 20 miNs overlapped with regions of fibroblasts marked by H3K9me3/H3K27me3, which were heterochromatic regions in fibroblasts. Whereas H3K27ac/H3K4me1 marked euchromatic regions in fibroblasts overlapped with 16,207 peaks that were closed in miNs (see e.g., FIG. 6H). GO enrichment analysis of genes associated with open chromatin regions in miNs marked by H3K9me3 or H3K27me3 in fibroblasts resulted in neuronal differentiation and function-related terms (see e.g., FIG. 7B). In contrast, regions that lose chromatin accessibility in miNs were enriched with non-neuronal terms (see e.g., FIG. 7D). These results demonstrate the surprisingly potency of miR-9/9*-124 to open heterochromatin regions needed for neuronal development and to close enhancer regions that preexist in fibroblasts and are not active in neurons. The present results collectively provide an unprecedented demonstration that microRNAs can change chromatin architecture to promote neuronal and repress fibroblast fates during the direct conversion of human fibroblasts to neurons.

(J) Chromatin Remodeling is Required for Direct Conversion

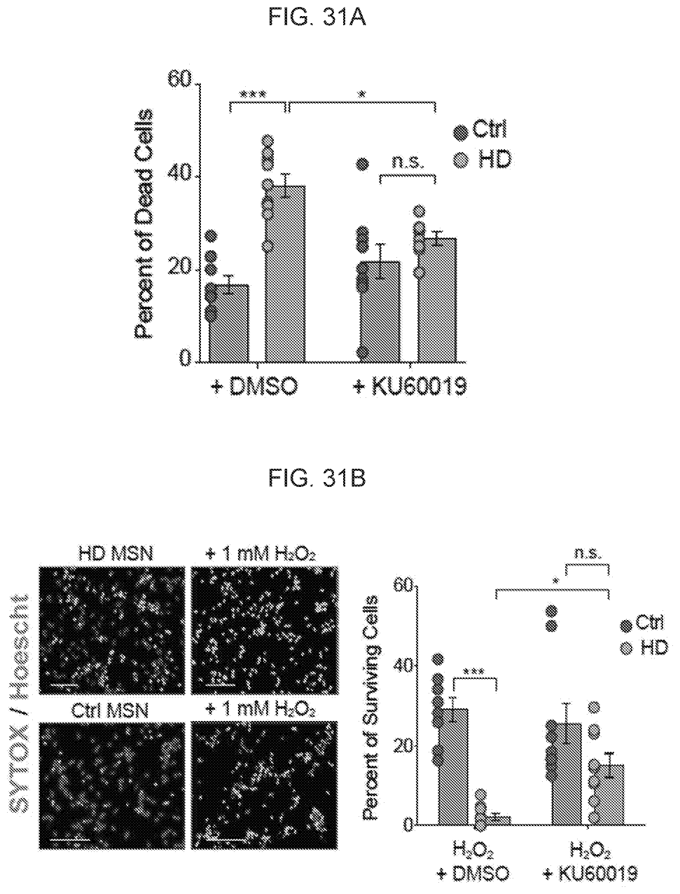

To determine if chromatin changes were necessary for cell fate conversion the expression BRG1 was knocked down. BRG1 is a core component of BAF chromatin remodeling complex whose reduced function has been shown to collapse the overall chromatin architecture (Kadoch et al., 2017). After 20 days of neuronal conversion, loss of BRG1 markedly decreased the amount of MAP2 positive cells when compared to a control shRNA (FIG. 31A). ATAC-seq revealed regions which failed to open in response to miR-9/9*-124 in the absence of BRG1 (FIG. 31A). These regions were associated with neuronal GO terms in contrast to the fibroblast-related GO terms associated with regions that failed to close (FIG. 31D). These data demonstrate the requirement of chromatin remodeling in cell fate conversion. Collectively, our transcriptome and ATAC-seq results provide mechanistic insight into the possibility of deriving additional clinically relevant neuronal subtypes through miRNA-mediated conversion in addition to cortical and striatal medium spiny neurons (Victor et al., 2014; Yoo et al., 2011).

(K) Instructing miRNA-Induced Neurogenic State to Motor Neuron Fate

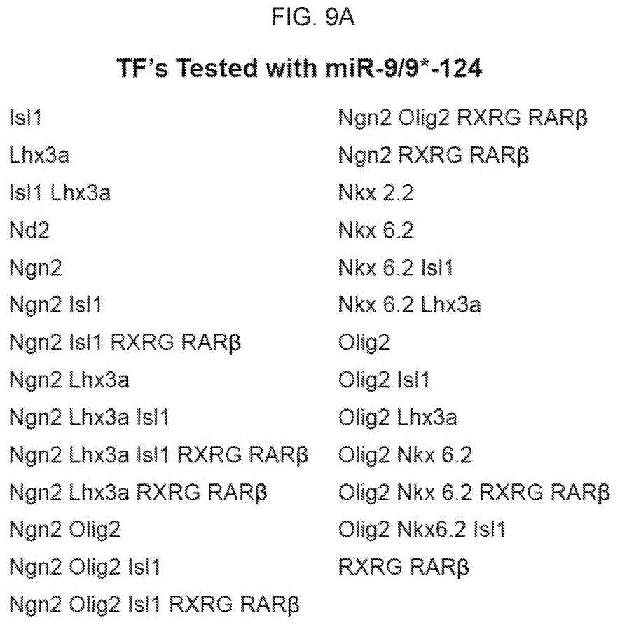

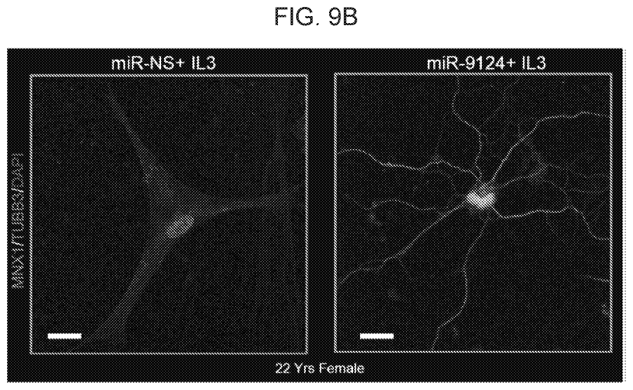

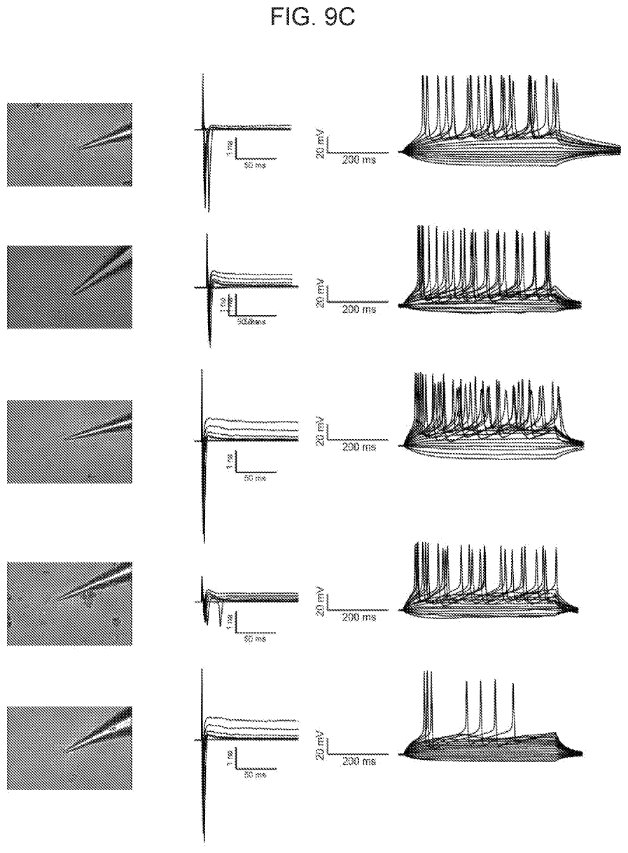

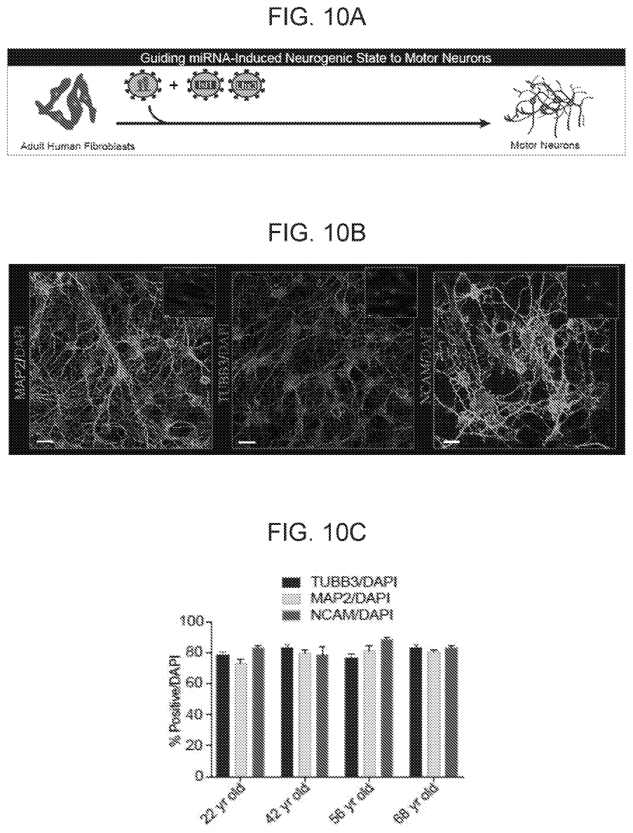

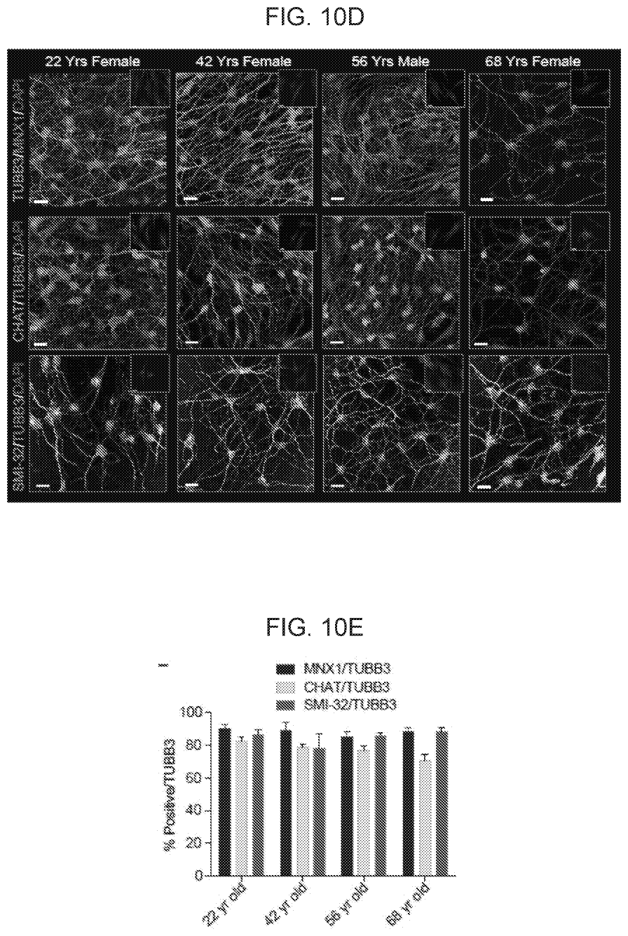

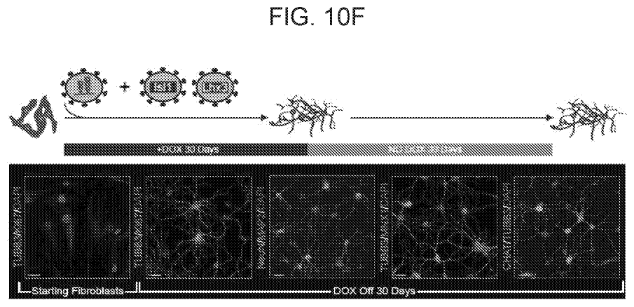

The deposition and removal of nucleosomes along regulatory elements within DNA inhibits or enables the binding of TFs, simultaneously facilitating and reinforcing cell-type specific gene expression programs (Jiang and Pugh, 2009). It was noted chromatin regions in miNs with enhanced accessibility were proximal to MNX1 and choline acetyl transferase CHAT, two of the hallmark genes expressed by motor neurons (Fonnum, 1973; Tanabe et al., 1998) (see e.g., FIG. 6I). Neither MNX1 nor CHAT mRNA levels were elevated in day 30 miNs after miR-9/9*-124 directed neuronal conversion, it was therefore hypothesized the open chromatin would facilitate expression of these genes in response to motor neuron specific TFs. To test this hypothesis a panel of TFs that promote motor neuron identity (see e.g., FIG. 9A) were expressed in the background of ectopic miR-9/9*-124 expression and assayed for MAP2 and MNX1 expression by immunostaining (data not shown). While all combinations resulted in MAP2 positive cells, only ISL1 and LHX3 robustly led to the generation of MNX1-positive cells. Co-expressing LHX3 and ISL1 with miR-9/9′-124 in adult human fibroblasts from 22-, 42-, 56- and 68-year old donors resulted in MAP2, TUBB3, and NCAM-positive cells with complex neuronal morphologies (see e.g., FIG. 10A and FIG. 10B). Approximately 80% of all cells positive for DAPI staining were positive for TUBB3, MAP2, and NCAM expression (see e.g., FIG. 10C). The majority of converted cells displayed nuclear staining of MNX1 (˜85% of TUBB3 positive cells in each line, FIG. 10D and FIG. 10E). Similarly, cytoplasmic CHAT protein and SMI-32 (a neurofilament protein found in motor neurons) were detected in ˜80% of TUBB3 positive cells within each age group (see e.g., FIG. 10D and FIG. 10E).

Interestingly, ISL1 and LHX3 alone were not sufficient to induce neuronal conversion when co-expressed with a non-specific miRNA (miR-NS) (see e.g., FIG. 96). This result supports the notion that miR-9/9*-124 is necessary for the subtype-specifying activities of ISL1 and LHX3. Motor neuron conversion induced by miR-9/9*-124 plus ISL1 and LHX3 was stable, displaying neuronal morphologies, cell cycle exit and motor neuron marker expression for 30 days after doxycycline removal (see e.g., FIG. 10F).

(L) Electrophvsioloaical properties of Moto-miNs

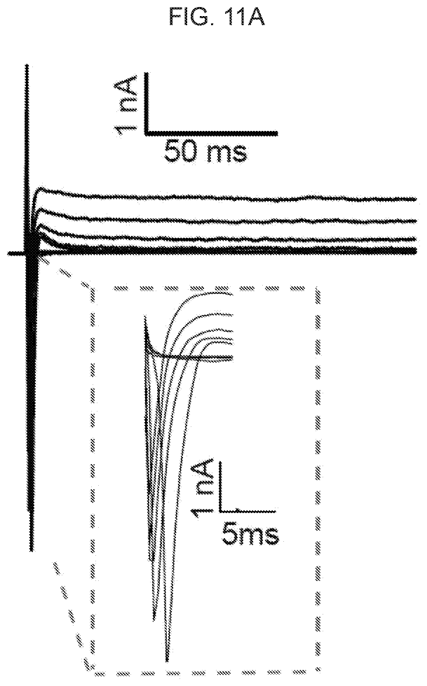

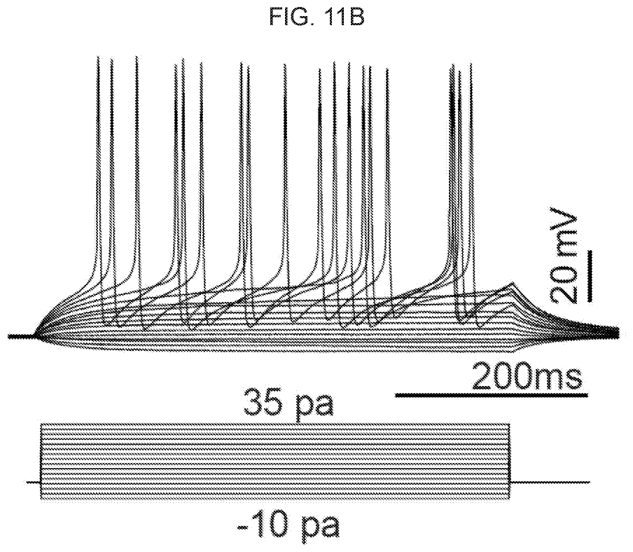

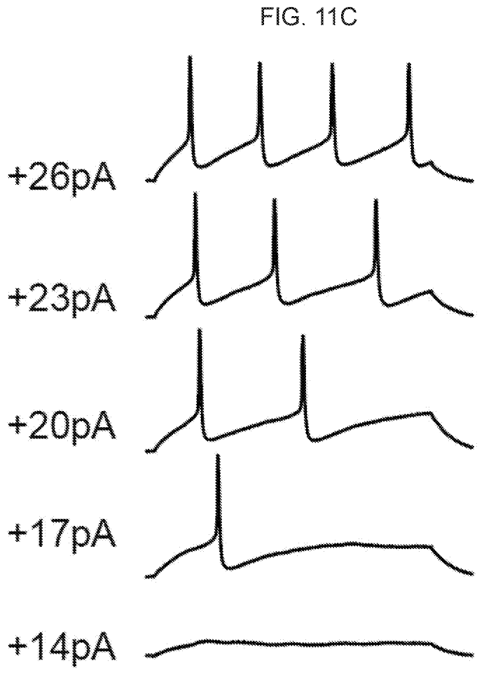

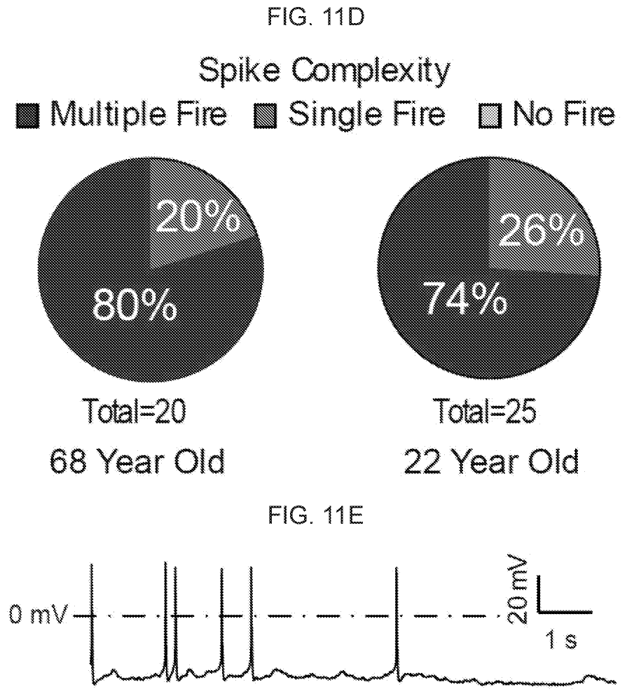

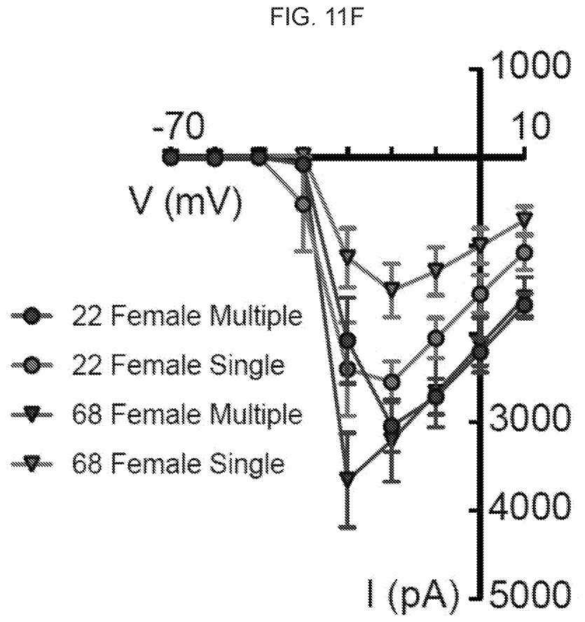

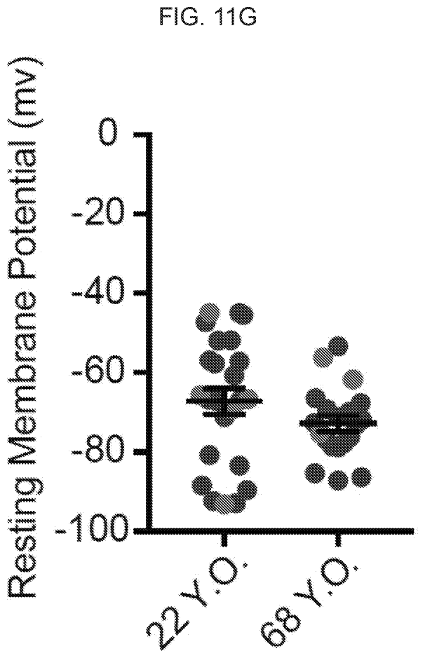

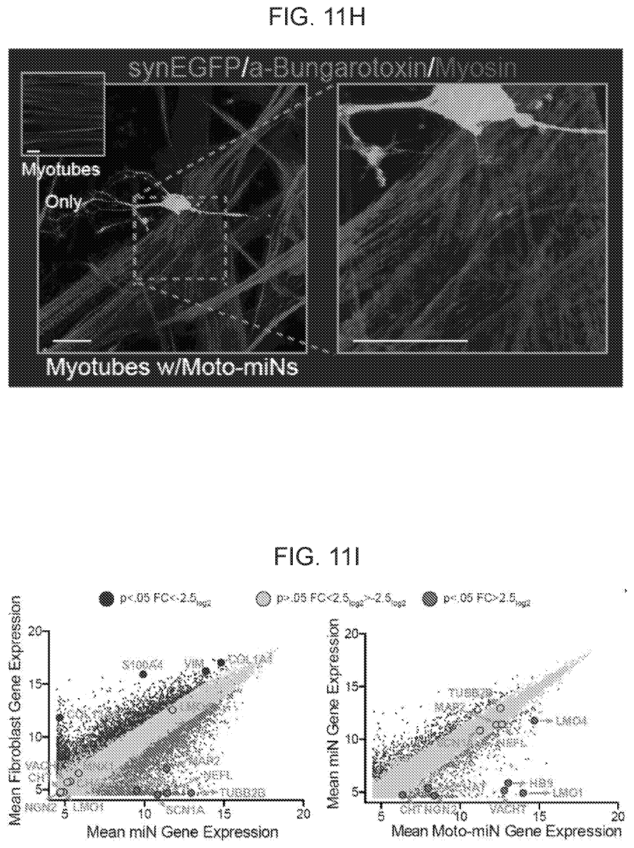

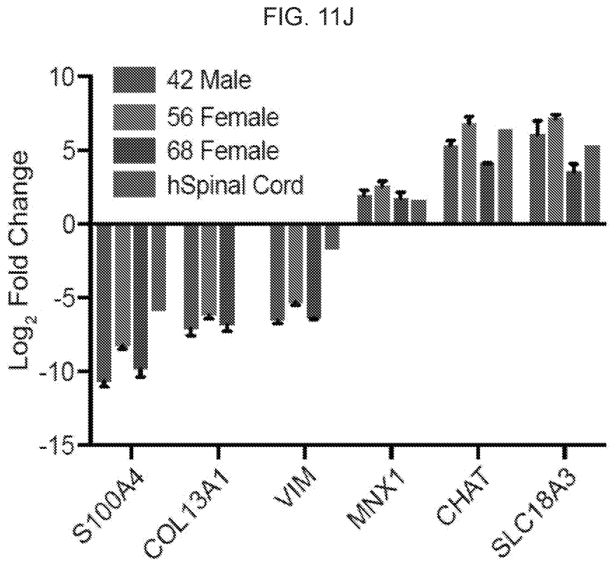

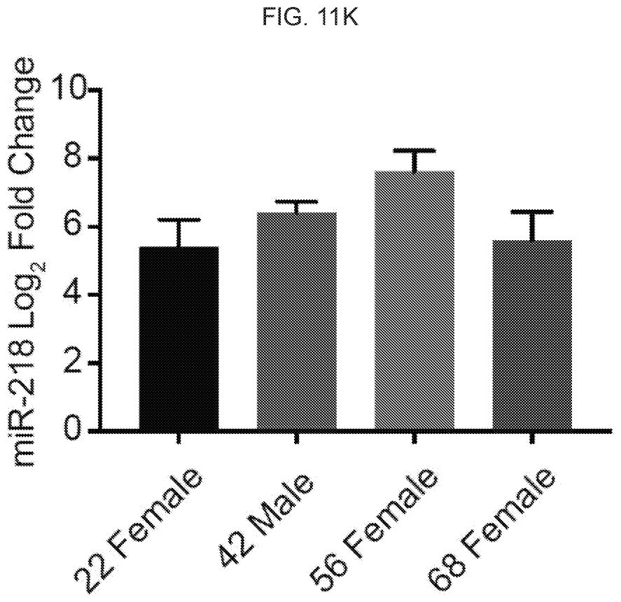

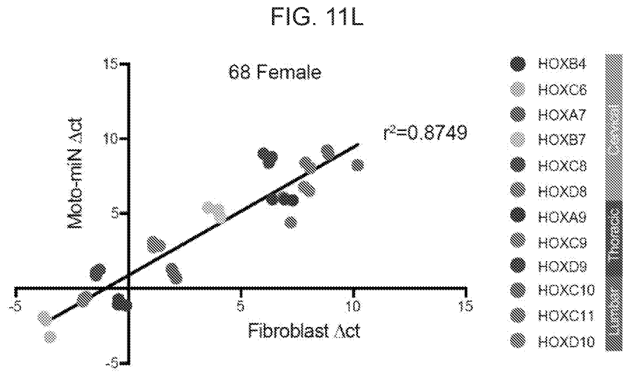

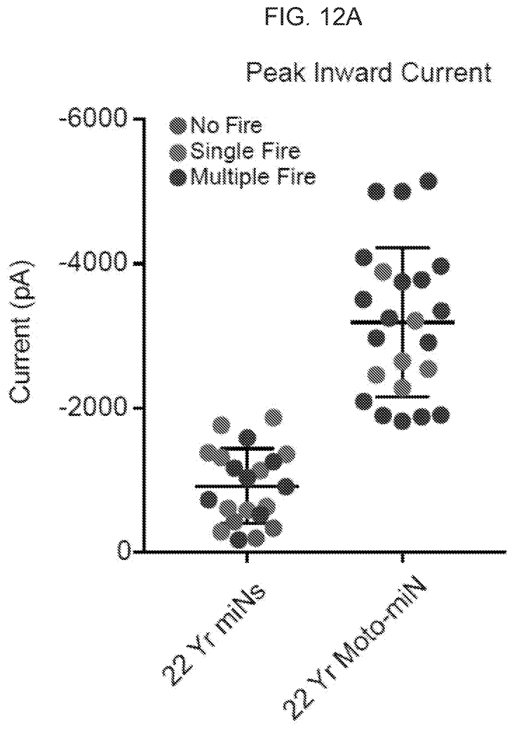

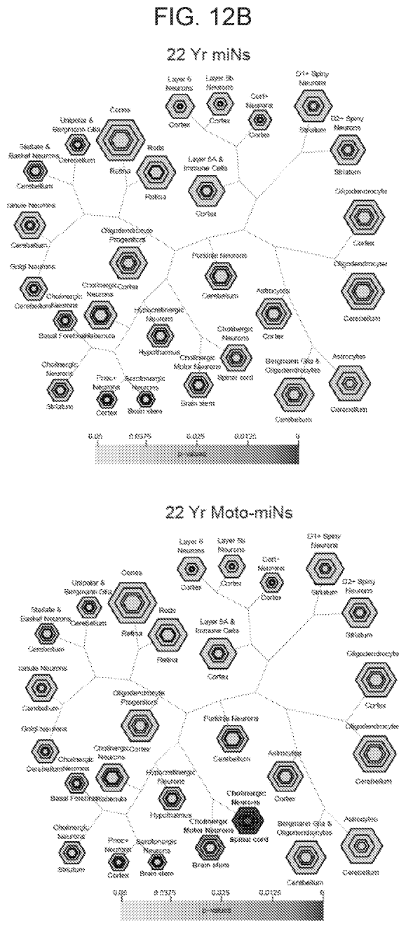

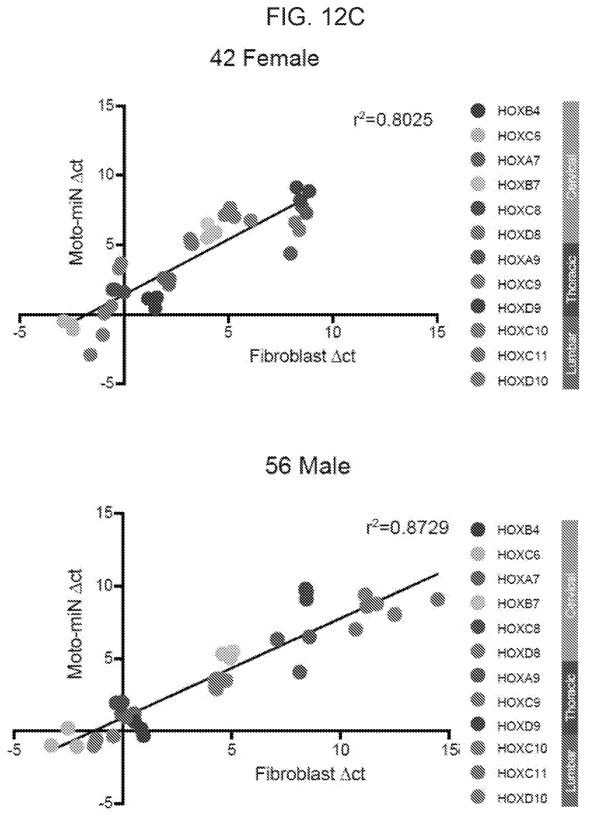

Motor neurons produced from fibroblasts by co-expression of miR-9/9*-124, ISL1, and LHX3 (Moto-miNs) demonstrated robust inward and outward currents in response to depolarizing steps (see e.g., FIG. 11A) and displayed action potential trains through the injection of step-wise depolarizations (see e.g., FIG. 11B and FIG. 9C). The visualization of single traces recorded at individual current steps revealed the characteristic hyperpolarization following each action potential seen in mature neurons (see e.g., FIG. 11C). In order to assess the percentage of functionally mature Moto-miNs, 45 randomly chosen Moto-miNs were patched from 22-year-old and 68-year-old donors. All Moto-miNs fired action potentials (APs). Most cells fired multiple APs (80% n=20 and 74% n=25), while single APs were observed in approximately 20-25% of the patched cells (see e.g., FIG. 11D). Gap-free recordings in current clamp mode revealed cells capable of firing spontaneous action potentials (see e.g., FIG. 11E) demonstrating the excitability of these cells. Similar I-V curve relationships were observed between donors and firing patterns (see e.g., FIG. 11F). Peak inward currents were substantially higher than those observed in miNs (see e.g., FIG. 12A). Lastly, resting membrane potentials in all Moto-miNs tested were hyperpolarized (see e.g., FIG. 11G; 22 yr old, −67.2 mV±3.3 mV; 68 yr old, −72.8 mV±2.0 mV, S.E.M.). Coupled with the increased proportion of cells that fire multiple APs these data suggest that the addition of ISL1 and LHX3 to miR-9/9*-124 produced more mature neurons than exposure to miR-9/9*-124 alone.

The ability of motor neurons to control voluntary muscle movement stems from their ability to form neuromuscular junctions (NMJs), unique synapses formed between motor neurons and muscle cells. The formation of neuro-muscular junctions (NMJs) was visualized through the co-localization of EGFP-labeled Moto-miNs, Alexa-fluor-594, Bungarotoxin (BTX, a toxin that binds to the nicotinic acetylcholine receptor (AChR) of NMJs), and myosin heavy chain. BTX puncta were not observed in the absence of Moto-miNs (see e.g., FIG. 11H, left inset). In contrast, Moto-miNs were able to induce characteristic BTX-clustering in close apposition with EGFP labeled neurons and myotubes (see e.g., FIG. 11H) indicating the formation of putative NMJs.

(M) Transcriptional Profiling of Moto-miNs

To fully characterize the acquisition of a motor neuron fate and assess the contribution of ISL1 and LHX3, the transcriptome of 22-yr-old starting fibroblasts, miNs and Moto-miNs were profiled by microarray. The loss of fibroblast gene expression (for example, S100A4, VIM and COL13A1) was again observed, and the gain of a pan-neuronal identity (for example, MAP2, NEFL, SNAP25 and SCN1A) 35 days after the expression of miR-9/9*-124 (see e.g., FIG. 11I, left). Expression of motor neuron-specific genes was not significantly different between starting fibroblasts and miNs. While the addition of ISL1 and LHX3 to miR-9/9*124 did not significantly change the expression of pan-neuronal genes when compared to miNs (see e.g., FIG. 11I, right), ISL1 and LHX3 selectively activated key motor neuron genes including MNX1, CHAT, VACHT, LMO1, and LMO4 (see e.g., FIG. 11I, right). The loss of fibroblast identity and gain of motor-neuron identity in Moto-miNs derived from 42-, 56- and 68-year-old donors was further validated by qRT-PCR using RNA from human spinal cord as a positive control (see e.g., FIG. 11J). In addition, the observed upregulation of miR-218, a recently identified motor neuron-specific microRNA was analyzed and a dramatic upregulation of miR-218 in Moto-miNs was observed (see e.g., FIG. 11K).

Next, the cell type-specific enrichment analysis tool (CSEA) was used (Xu et al., 2014) to test whether the gene expression profile within each population of miNs and Moto-miNs would be associated with distinct subtypes of in vivo neurons. When queried with a gene list, CSEA identifies neuronal subtypes that show significant enrichment in genes from the input list through curated transcriptomic data. The CSEA analysis of the 100 most enriched genes within Moto-miNs identified two subtypes, brainstem motor neurons and spinal motor neurons, to be significantly associated. No subtype specificity found in the miN transcriptome (see e.g., FIG. 12A and FIG. 12B). This unbiased bioinformatics approach further supports the motor neuron identity of converted Moto-miNs.

Lastly, HOX gene expression patterns were compared by qRT-PCR between starting fibroblasts and Moto-miNs derived from the conversion. Interestingly, a high correlation (R²=0.88) was observed between the expression levels of HOX genes before and after conversion within each of the defined spinal cord regions tested, indicating Moto-miNs retain the positional identity that existed in original fibroblasts (see e.g., FIG. 11L and FIG. 12C).

(N) Transcriptional Activation of ISL1 and LHX3 Genomic Targets

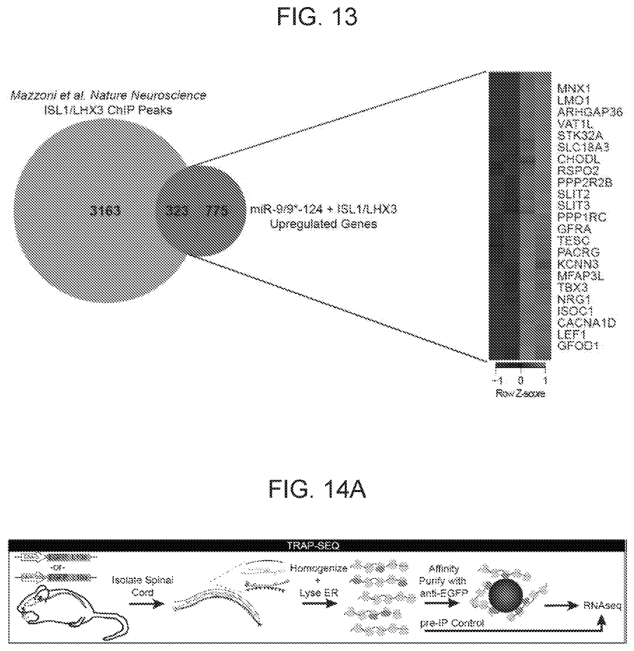

An alternative approach for generating motor neurons is forced expression of NGN2, ISL1, and LHX3 in human embryonic stem cells (ESCs) (Mazzoni et al., 2013). The genomic targets of ISL and LHX3 identified by Mazzoni et al. were compared through ChIP-seq to genes whose expression increases in Moto-miNs compared to miNs. Surprisingly, a large cohort of overlapping genes (323) were identified that included numerous hallmark motor neuron markers (see e.g., FIG. 13). This result suggests a core ISL1/LHX3 gene regulatory network that underlies the specification of diverse cellular states towards motor neurons.

(O) Comparison of Moto-miNs to Endogenous Spinal Cord Motor Neurons

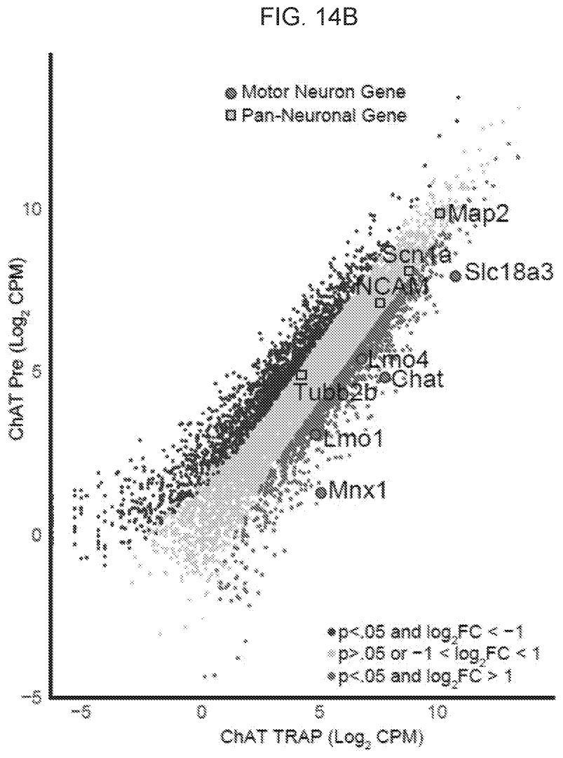

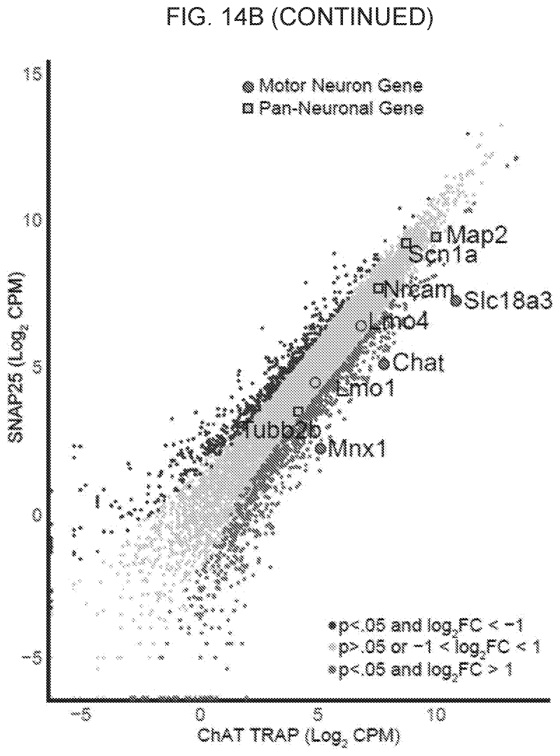

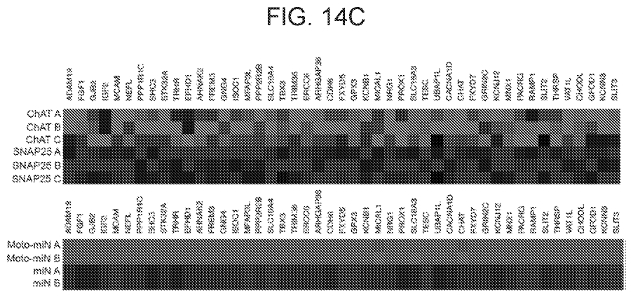

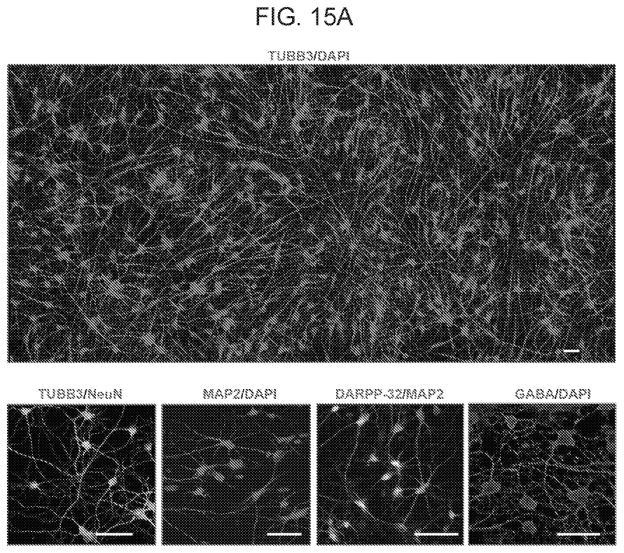

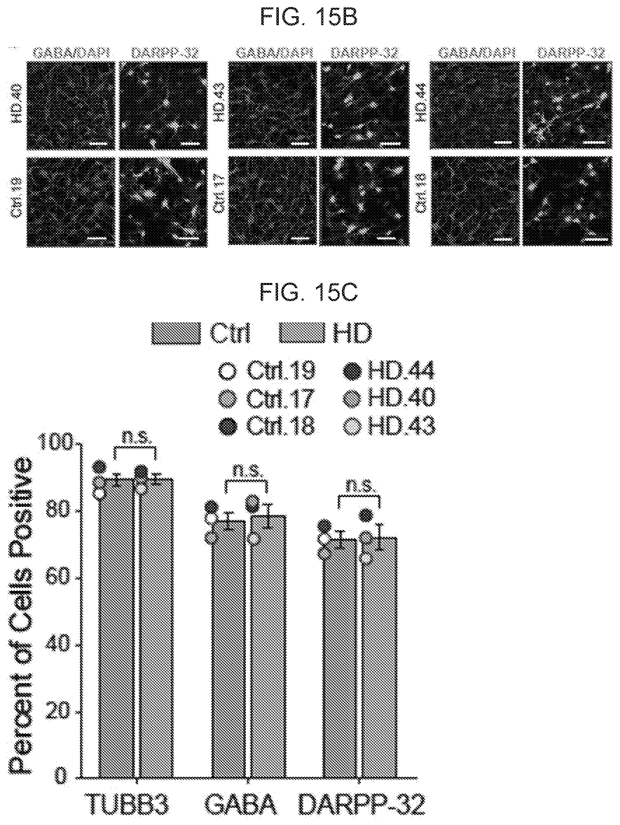

The obvious difficulty in obtaining a pure population of motor neurons within human individuals prevents direct transcriptional comparisons between Moto-miNs and their human in vivo counterpart. Therefore, Moto-miNs were directly compared to fully differentiated in vivo mouse motor neurons. To interrogate the gene expression of motor neurons within the large heterogeneity of cell-types present in the spinal cord, Translating Ribosomal Affinity Purification (TRAP) followed by RNA-seq was performed (see e.g., FIG. 14A). The use of two mouse lines expressing EGFP tagged ribosomes (one line under the pan-neuronal SNAP25 promoter, and the other through the CHAT promoter), enabled the enrichment and subsequent sequencing of actively transcribed mRNA in all neurons and motor neurons within the spinal cord. The transcriptome of the entire spinal cord was also profiled as an additional‘pre-IP’ control. Comparisons between CHAT pre-IP controls and CHAT-TRAP transcripts confirmed significant enrichment of motor neuron markers (see e.g., FIG. 14B, top) by the TRAP procedure. This comparison, however, does not distinguish between pan-neuronal versus motor neuron specific transcripts. Therefore to further separate motor neuron enriched genes from common neuronal genes CHAT-TRAP and SNAP-25 datasets were first normalized to their pre-IP controls, then directly compared the normalized gene expression values. This analysis revealed the co-expression of pan-neuronal genes such as MAP2, NRCAM, SCN1A and TUBB3 and highly enriched for motor neurons transcripts such as SLC18A3 (VACHT), CHAT, and MNX1 (see e.g., FIG. 14B, bottom). This dataset also serves as a resource for genes enriched in spinal cord neurons over all SNAP25 expressing neurons within the spinal cord. To that end, differentially expressed mouse spinal cord motor neuron genes were next compared to genes enriched in Moto-miNs over miNs. A significantly larger overlap was observed between Moto-miNs and mouse motor neurons than expected by chance (Fisher's exact test p=3.522e-06), indicating that Moto-miNs and endogenous mouse motor neurons utilize similar genetic networks.

This includes expression of canonical motor neuron markers such as, SLIT2 and SLIT3, host genes for the motor neuron specific miRNA, miR-218 (Amin et al., 2015) (FIG. 14C). Altogether, this in-depth analyses of Moto-miNs at cellular, functional, and transcriptomic levels confirm that co-expression of miR-9/9*-124 and ISL1/LHX3 can directly convert adult fibroblasts into spinal cord motor neurons.

### DISCUSSION

The process by which a fully differentiated fibroblast of mesodermal origin is directly converted into a functional neuron—a highly specialized cell normally arising from neuroectoderm—has remained largely enigmatic. Exploring the mechanism of cell fate conversion, and determining the manner in which reprogramming factors synergize, presents a unique opportunity in the study of lineage commitment, and provides a molecular foundation for choosing factors that guide conversion towards clinically relevant cell types.

In this study, the extensive neurogenic potential of miR-9/9* and miR-124 has been dissected, two brain-enriched miRNAs that when ectopically expressed in adult human fibroblasts directly evoke a neuronal state characterized by morphological changes, chromatin remodeling and DNA methylation, neuronal protein expression, and importantly, the adoption of intrinsic functional properties. The identification of miRNA-induced neurogenic state has provided molecular insights into how multiple neuronal subtypes can be generated from patient fibroblasts for modeling neurological diseases. The observation that miRNAs alone can stimulate direct conversion—leading to epigenetic, transcriptome and functional remodeling—simultaneously demonstrated the substantial neurogenic information embedded in small non-coding RNAs.

MiRNA-mediated neuronal conversion appears to be distinct from current models of cell fate reprogramming. Two models of lineage reprogramming have been proposed: one based on transcription factor cooperativity and positive feedback loops (Jaenisch and Young, 2008; Soufi et al., 2012; Vierbuchen and Wemig, 2012), and the other proposes that the “on-target” pioneer activity of a TF initiates and enables additional TFs to assist in cellular conversion (Wapinski et al., 2013). In stark contrast, canonical gene regulation by miRNAs requires the removal of information through translational repression and transcript degradation. This mode of repression in conjunction with the multitude of anti-neurogenic genes targeted by miR-9/9*-124 suggests miRNA-mediated reprogramming acts through an alternative mechanism. It is currently believed that miR-9/9*-124 expression in non-neuronal somatic cells initiates gradual, yet active changes in the activities of multiple chromatin modifiers while simultaneously repressing anti-neuronal genes and activating neuronal genes culminating in a binary cell-fate switch. This model is supported by the rapid cell cycle exit observed upon ectopic miR-9/9*-124 expression, the subsequent neuronal switching within chromatin modifiers, steady increase in epigenetic and transcriptional changes, and the time scale in which conversion takes place.

Chromatin Remodeling Accompanies Cell Fate Conversion.

In this study, the surprising potency of miR-9/9*-124 for remodeling chromatin and altering DNA methylation was revealed. Surprisingly, preexisting neuronal loci within the heterochromatic regions in human fibroblasts opened up in response to miR-9/9*-124. These data suggest the robustness of miRNA-mediated reprogramming observed in human cells could stem from their ability to induce epigenetic changes. Cellular processes and identity are governed by the cumulative action of multiple levels of genome regulation and it is unlikely a single genetic component downstream of miR-9/9*-124 mediates these changes and ultimately cell fate conversion. For example, almost every level of epigenetic remodeling participates in the induction of pluripotency (Takahashi and Yamanaka, 2016). Instructions operating through multiple levels of genetic and epigenetic regulation are likely required for true cell-fate conversion. The thorough characterization of miR-9/9*-124 induced transdifferentiation of human fibroblasts into functional neurons highlights molecular processes that are critical to cell fate conversion.

A Modular Neuronal State.

Importantly, the plastic neuronal platform presented here affords modularity to direct conversion. The synergism between miR-9/9*-124 and TFs was shown by generating a neuronal population highly enriched with spinal cord motor neurons from human adult fibroblasts through the coexpression of miR-9/9*-124, ISL1, and LHX3. Because MNs are a clinically relevant subtype affected in Amyotrophic Lateral Sclerosis and Spinal Muscular Atrophy, the robustness and specificity of neuronal conversion employing miRNAs and motor neuron TFs may pave the way towards generating patient-specific MNs for disease modeling. Unfortunately, the potent reprogramming capabilities of miR-9/9*-124 are likely restricted to neuronal identities. The brain-restricted expression of both miR-9/9* and miR-124 coupled with the data presented here, suggests this reprogramming paradigm is restricted to generating cells within the neuronal compartment, and it is unlikely that TFs important in other specialized non-neuronal lineages would enable miR-9/9*-124-mediated reprogramming towards the corresponding cell types.

The plastic neuronal platform presented here affords modularity to direct cell fate conversion. Numerous studies in developmental neuroscience have identified subtype-specific TFs or terminal selector genes that could be incorporated in neuronal reprogramming technology. Yet, identifying molecules capable of overcoming the cell-fate barrier present in human somatic cells and eliciting a permissive environment in which terminal selector genes can act has proven to be challenging. Here, this property was demonstrated by generating a neuronal population highly enriched in spinal cord motor neurons from human adult fibroblasts through the coexpression of miR-9/9*-124, ISL1, and LHX3. Because motor neurons are the major neuronal subtype affected in Amyotrophic Lateral Sclerosis (ALS) and Spinal Muscular Atrophy (SMA), the robustness and specificity of neuronal conversion employing miRNAs and motor neuron TFs may pave the way towards generating patient-specific MNs for disease modeling.

### Experimental Procedures

(i) Plasmid Construction and Virus Production.

Complementary cDNA was generated from adult human spinal cord (Clontech) from which individual motor neuron transcription factors were subcloned into the N174 (Addgene 60859) and N106 (Addgene, 66808) lentiviral vectors using standard techniques. Lentivirus was produced in 2931e cells plated in 10 cm dishes (6.5×10⁶ cells per dish) via polyethylineimine (48 μL of 2 mg/mL, Polysciences) assisted transfection of 3^{rd} generation packaging vectors (1.5 μg pMD2.G, Addgene, 12259 and 4.5 μg psPAX2 Addgene, 12260), and 6 μg of lentiviral backbone plasmid (e.g. pT-BCL-9/9*-124; Addgene, 60859) 16 hours after initial plating. Media was changed the next day. After 2 days, media was harvested, filtered through a 0.45 μm polyethersulfone (PES) syringe filter and then concentrated by centrifugation at 70,000×G for 2 hours at 4C. Virus collected from a single 10 cm dish was resuspended in 1 mL of sterile PBS then aliquoted and stored at −80° C. Before each transduction, virus aliquots were spun at 5,000×G for 5 minutes at 4° C. to remove debris. Control vector expressing non-specific (NS) miRNA and BCL-XL was generated previously (Victor et al., 2014).

(ii) Cell Culture

Adult and Neonatal Human Fibroblasts were obtained from commercial sources and maintained in fibroblast media comprised of Dulbecco's Modified Eagle Medium (Invitrogen) supplemented with 15% fetal bovine serum (Life Technologies) 0.01% β-mercaptoethanol (Life Technologies), 1% non-essential amino acids 1% sodium pyruvate, 1% GlutaMAX, 1% 1M HEPES buffer solution and 1% penicillin/streptomycin solution (all from Invitrogen) and never passaged more than 15 times. Fibroblasts utilized in this study: 1 yr (PCS-201-010, ATCC), 22 yr (GM02171, NIGMS Human Genetic Cell Repository at the Coriell Institute for Medical Research) 42 yr (F09-238, Washington University in St. Louis School of Medicine iPSC core facility), 56 yr (AG04148, NIA Aging Cell Repository at the Coriell Institute for Medical Research), 68 yr (ND34769, NINDS Cell Line Repository at the Coriell Institute for Medical Research)

(iii) Direct Conversion

To initiate direct conversion, 1.8×10⁶ cells were seeded onto Costar 6 well cell culture vessels (Corning; 300,000 cells/well). The following day, each plate was transduced with the following reprogramming cocktail: 750 μL of concentrated lentivirus containing the reverse tetracycline-controlled transactivator (rtTA; Addgene, 66810) and 500 μL of virus containing pT-BCL-9/9′-124 or pT-BCL-9/9-124 and 500 μL of each individual TF driven by the EF1α promoter, and polybrene (8 μg/mL; Sigma-Aldrich) all diluted up to 18 mL (3 mL per well) then spinfected at 37° C. for 30 minutes at 1,000×G using a swinging bucket rotor. The following day media was changed to fresh fibroblast media (2 mL per well) supplemented with doxycycline (Dox; Sigma Aldrich, 1 μg/mL). After 2 days, fresh fibroblast media was changed and supplemented with Dox and antibiotics for respective vectors (Puromycin, 3 μg/ml; Blasticidin 5 μg/ml; Geneticin, 400 μg/mL; all from Invitrogen). Five days post-transduction cells were replated on to poly-omithine/laminin/fibrobnectin (PLF) coated glass coverslips. Before PLF coating, glass coverslips were acid treated according to (Richner et al., 2015). For each well of a 6 well plate, cells were first washed 2× with 1 mL sterile PBS, then 320 μL of 0.25% Trypsin (Gibco) was added to each well then placed in an incubator. Cells were monitored every 2 minutes, as soon as cells began to detach (no more than 6 minutes) 1 ml of MEF media supplemented with 1 μg/mL Dox was added to each well. One by one, each well was gently triturated three times to remove remaining attached cells then transferred to a sterile 1.5 mL eppendorf tube. Cells were then spun at 200×G for 5 minutes at 37° C. The supernatant was aspirated and cells were gently resuspended in 300 μL MEF media supplemented with Dox. Cells were then drop-plated onto either 18 mm (150 μL per/c.s.; placed in 12 well plate) or 12 mm (60 μL per c.s.; placed in 24 well plate) coverslips. Cells were left to settle for 15 minutes in an incubator then each well was flooded with MEF media supplemented with 1 μg/mL Dox. The following day media was then changed to Neuronal Media (Sciencell) supplemented with Dox, valproic acid (1 mM; EMD Millipore) dibutyryl cAMP (200 μM; Sigma-Aldrich), BDNF, NT-3, CNTF, GDNF (all 10 ng/ml, Peprotech), and Retinoic Acid (1 μM: Sigma-Aldrich) and antibiotics for each vector. Dox was replenished every two days and half the media was changed every 4 days. Drug selection was halted 14 days into conversion. A diagram of the reprogramming protocol is available in FIG. 2.

For DNA methylation profiling, 1.8×10⁶ human neonatal fibroblasts were seeded onto 10 cm plates (Corning). The following day, each plate was transduced with 10 ml of un-concentrated lentivirus containing a doxycycine inducible miR-9/9*-124 vector (Victor et al., 2014) and polybrene (8 μg/mL; Sigma-Aldrich). The following day media was changed to fresh fibroblast media (2 mL per well) supplemented with Dox. After 2 days, fresh fibroblast media was changed and supplemented with Dox and antibiotics for respective vectors (see TABLE 1). Seven days post-transduction, cells were first washed 2× with 3 mL sterile PBS, then 1 ml of 0.25% Trypsin (Gibco) was added to each plate then placed in an incubator. Cells were monitored every 2 minutes, as soon as cells began to detach (no more than 6 minutes), 4 ml of MEF media supplemented with 1 μg/mL Dox was added to each plate. Cells were transferred to Primaria modified 10 cm plates (Corning) and 5 ml fresh fibroblast media supplemented with 1 μg/mL Dox was added to a final volume of 10 ml. The following day media was changed to Neuronal Media (Sciencell) supplemented with Dox, valproic acid (1 mM; EMD Millipore) dibutyryl cAMP (200 μM; Sigma-Aldrich), BDNF, and NT-3 (all 10 ng/ml, Peprotech), Retinoic Acid (1 μM; Sigma-Aldrich), and 4% FBS. Dox was replenished every two days and half the media was changed every 4 days.

(iv) Myotube Differentiation

Human myotubes were generated by differentiating human myoblasts using defined culture conditions (Steinbeck et al., 2016). Briefly, human skeletal myoblasts were cultured according to manufacturer's recommendations (HSMM; CC-2580, Lonza) then were plated on matrigel (0.1 mg/mL) coated 12 mm glass coverslips at a density of 80,000 cells/well. The following day HSMM's were differentiated by switching media to skeletal muscle differentiation media comprised of a 1:1 mixture of DMEM F12 (Gibco) and Complete Neuronal Media+2% Horse Serum (Gibco). Every 2 days % of the media was replaced with fresh differentiation media. After 10-14 days of differentiation, day 14 Moto-miNs labeled with synapsin-eGFP via lentiviral transduction were replated onto myotubes at a 1:1 ratio (i.e. one 12 mm Moto-miN coverslip was replated on top of a 12 mm myotube coverslip). The following day media was changed to complete neuronal media and cells were cultures for 2 weeks. Dox was replenished every two days and half the media was changed every 4 days. After two weeks cells were fixed with 4% paraformaldehyde and processed for immunocytochemistry.

(v) Immunofluorescence and Cell Counting

Cells were fixed using 4% formaldehyde for 18 minutes at room temperature (RT) then blocked and permeabilized for one hour at RT in PBS containing 0.3% Triton-X100, 5% bovine serum albumin (Sigma-Aldrich), and 2% of either goat or donkey serum (Sigma-Aldrich). Primary antibodies were incubated overnight at 4° C. in blocking buffer. Cells were then washed 3× then incubated with secondary antibodies conjugated to either Alexa-488, -594 or -647, for one hour at RT. The following antibodies were used for immunostaining: MAP2 (Sigma-Aldrich, 1:500), TUBB3B (Covance, 1:7000), NeuN (AVES, 1:300), SCN1A (Sigma-Aldrich, 1:300) ANKG (NeuroMAB, 1:1000) SV2 (DSHB, 1:250), HB9 (DSHB, 1:200) CHAT (Millipore, 1:100) SMI-32 (Biolegend, 1:2000) Ki-67, (Abcam, 1:200), Myosin (DSHB, 1:50), a-Bungarotoxin (ThermoFisher, 1:200), NCAM (eric1) (Santa-Cruz, 1:100). Images were obtained on a Leica SP-2 Confocal Microscope. Quantifications were performed on at least 10 random fields of view in duplicate experiments.

(vi) Electrophysiology Whole-cell patch-damp recordings were performed 35-40 days post-transduction. Data was acquired using pCLAMP 10 software with multiclamp 700B amplifier and Digidata 1550 digitizer (Molecular Devices). Electrode pipettes were pulled from borosilicate glass (World Precision Instruments) and typically ranged between 5-8 MO resistance. Intrinsic neuronal properties were studied using the following solutions (in mM): Extracellular: 140 NaCl, 3 KCl, 10 Glucose, 10 HEPES, 2 CaCl₂ and 1 MgCl₂ (pH adjusted to 7.25 with NaOH). Intracellular: 130 K-Gluconate, 4 NaCl, 2 MgCl₂, 1 EGTA, 10 HEPES, 2 Na-ATP, 0.3 Na-GTP, 5 Creatine phosphate (pH adjusted to 7.5 with KOH). Membrane potentials were typically kept at −65 mV. In voltage-clamp mode, currents were recorded with voltage steps ranging from −20 mV to +90 mV. In current-clamp mode, action potentials were elicited by injection of step currents that modulated membrane potential from −10 mV to +35 mV. Data was collected in Clampex and initially analyzed in Clampfit (Molecular Devices). Further analysis was done in GraphPad Prism 7 (GraphPad Software). Liquid junction potential was calculated to be 15.0 mV and corrected in calculating resting membrane potential according to previously published methods (Barry, 1994).

(vii) RNA-Seq Library Preparation and Sequencing

Day 30 miNs and starting human adult fibroblasts (22 yr old) were extracted by RNeasy plus micro kit (Qiagen). The RNA samples with >9.5 of RIN based on a 2100 Bioanalyzer were used for RNA-Seq library preparation. Library preparation and sequencing were performed by Genome Technology Access Center in Washington University School in St. Louis. Briefly mRNA was isolated by using SMARTer Ultra Low RNA Kit for Illumina sequencing (Clontech). All cDNA libraries, based on two biological replicates for each condition, were sequenced on Illumina Hi-Seq 2500 with single-end 50 bp read length.

(viii) RNA-Seq Data Analysis

More than 35 million reads of each RNA-seq data were aligned to human genome assembly GRCh 37. For differential expression analysis, edgeR and limma were used. Genes with low read counts, regarded as genes not expressed at a biologically meaningful level were filtered out before read normalization. The cut-off for low read count was counts per million (CPM)<1 in at least any two samples across the experiment Reads for each sample were normalized by the edgeR method of trimmed mean of M-values (TMM). The quantitative difference of read counts between miNs and starting fibroblast samples were evaluated by carrying out limma and graphically represented by Glimma. Gene enrichment analysis for differentially expressed genes was performed using Metascape Gene Annotation and Analysis Resource tool.

(ix) MicroArray Analyses

Total RNA was extracted from miNs and Moto-miNs derived from 22 yr old donor fibroblasts alongside corresponding starting fibroblast controls using TRIzol (Thermo Fisher Scientific, Waltham, Mass.) according to the manufacturer's instruction and extracted using chloroform and ethanol precipitation. RNA quality was determined by the ratio of absorbance at 260 nm and 280 nm to be approximately 2.0. Samples for RNA microarray were then standardly prepped and labeled with Illumina TotalPrep kits (Thermo Fisher Scientific, Waltham, Mass.) for Agilent Human 4×44Kv1. Standard hybridization and imagine scanning procedure were performed according to the manufacturer's protocol at Genome Technology Access Center at Washington University School of Medicine, St. Louis. The intensity of the probes was imported into Partek and quantile normalized. Differentially expressed genes were identified using Partek with a cut-off of adjusted p-value <0.05 and over 2.5 log₂ fold expression change.

(x) Methylated DNA Immunoprecipitation Sequencing

MeDIP-seq was performed as in Maunakea et al. (Maunakea et al., 2010). Five micrograms of genomic DNA was sonicated to a fragment size of ˜100-400 bp using the Bioruptor sonicator (Diagenode). End-repair, addition of 3′-A bases and PE adapter ligation with 2 μg of sonicated DNA was performed according to the Illumina Genomic DNA Sample Prep Kit protocol. Adapter-ligated DNA fragments were size selected to 166-366 bp and purified by gel electrophoresis. DNA was heat denatures and then immunoprecipitated with 5-methylcytidine antibody (Eurogentec; 1 μg of antibody per 1 μg of DNA) in 500 μl of immunoprecipitation buffer (10 μM sodium phosphate, pH 7.0, 140 mM sodium chloride and 0.05% Triton X-100) overnight at 4° C. Antibody/DNA complexes were isolated by addition of 1 μl of rabbit anti-mouse IgG secondary antibody (2.4 mg ml⁻¹, Jackson Immunoresearch) and 100 μl protein A/G agarose beads (Pierce Biotechnology) for 2 h at 4° C. Beads were washed nine times with immunoprecipitation buffer and then DNA was eluted in TE buffer with 0.25% SDS and 0.25 mg ml⁻¹ of proteinase K for 2 h at 50° C. DNA was then purified with the Qiagen Qiaquick kit and eluted in 30 μl EB buffer. Ten microliters of DNA was used for a PCR-enrichment reaction with PCR PE Primers 1.0 and 2.0. PCR products were size selected (220-420 bp) and purified by gel electrophoresis. Methylated DNA enrichment was confirmed by PCR on known methylated (SNRPN and MAGEA1 promoters) and unmethylated (a CpG-less sequence on chromosome 15 and glyceraldehyde 3-phosphate dehydrogenase promoter) sequences. DNA libraries were checked for quality by Nanodrop (Thermo Scientific) and Agilent DNA Bioanalyzer (Agilent). Reads were aligned to hg19 using BWA and pre-processed using methylQA (an unpublished C program; available at http://methylqa.sourceforge.net/). Detailed library construction protocols for MRE-seq and MeDIP-seq are publically available at the NIH Roadmap Epigenomics project website (http://www.roadmapepigenomics.org/protocols/type/expermental/.

(xi) Methylation-Sensitive Restriction Enzyme Sequencing

MRE-seq was performed as in Maunakea et al. (Maunakea et al., 2010), with modifications as detailed below. Five parallel restriction enzyme digestions (Hpall, Bsh12361, Ssl(Acil) and Hin6l (Fermentas), and HpyCH41V (NEB)) were performed, each using 1 μg of DNA per digest for each of the samples. Five units of enzyme were initially incubated with DNA for 3 h and then an additional five units of enzyme were added to the digestion for a total of 6 h of digestion time. DNA was purified by phenol/chloroformisoamyl alcohol extraction, followed by chloroform extraction using phase lock gels. Digested DNA from the different reactions was combined and precipitated with one-tenth volume of 3 M sodium acetate (pH 5.2) and 2.5 volumes of ethanol. The purified DNA was size selected and purified (50-300 bp) by gel electrophoresis and Qiagen MinElute extraction. Library construction was performed as per the Illumina Genomic DNA Sample Prep Kit protocol with the following modifications. During the end-repair reaction, T4 DNA polymerase and T4 PNK were excluded and 1 μl of 1:5 diluted Klenow DNA polymerase was used. For the adapter ligation reaction, 1 μl of 1:10 diluted PE adapter oligo mix was used. Ten microliters from the 30 μl of purified adapter ligated DNA was used for the PCR enrichment reaction with PCR PE Primers 1.0 and 2.0. PCR products were size selected and purified (170-420 bp) by gel electrophoresis and Qiagen Qiaquick extraction. DNA libraries were checked for quality by Nanodrop (Thermo Scientific) and Agilent DNA Bioanalyzer (Agilent). Reads were aligned to hg19 using BWA and pre-processed using methylQA. MRE reads were normalized to account for differing enzyme efficiencies and methylation values were determined by counting reads with CpGs at fragment ends (Maunakea et al., 2010).

(xii) Differential DNA-Methylated Region Analysis

The M&M statistical model (Zhang et al., 2013), which integrates MeDIP-seq and MRE-seq data to identify differentially methylated regions between two samples was implemented with a window size of 500 bp and a q-value (false discovery rate (FDR)-corrected P-value) cutoff of 5e-2. This cutoff was determined from FIG. 5C, where only 1 DMR was detected at day 10 (miN day 10 vs. Ctrl day 10). For FIG. 5B, only regions that were considered DMRs (q-value <1e-5) at both day 20 (miN day 20 vs. Ctr day 20) and day 30 (miN day 30 vs. Ctr day 30) are displayed.

(xiii) GO Enrichment Analyses

DNA methylation GO analyses of MGI (Mouse Genome Informatics) expression (Smith et al., 2014) presented in FIG. 5D were performed using the GREAT package (McLean et al., 2010). Gene regulatory domains were defined by default as the regions spanning 5 kb upstream and 1 kb downstream of the TSS (regardless of other nearby genes). Gene regulatory domains were extended in both directions to the nearest gene's basal domain, but no more than a maximum extension in one direction. The top 100 most significant overlapping DMRs from day 20 (miN day 20 vs. Ctrl day 20) and day 30 (miN day 30 vs. Ctrl day 30) were used as input. For FIG. 5G, GO analyses were performed using Metascape (Tripathi et al., 2015) with a minimum enrichment of 1.5, a minimum overlap of 3, and a p-value cutoff of 0.01 using all demethylated or methylated DMRs at day 30 (miN day 30 vs. Ctrl day 30) that were either up- or downregulated, respectively, by RNA-seq at day 30 using a cutoff of 2.5 logFC.

(xiv) Genomic Features

DMRs from day 30 (miN day 30 vs. Ctrl day 30) were segregated into exons, introns, intergenic regions, 3′ UTRs, 5′ UTRs, non-coding regions, promoter-TSSs, and TTSs by using the annotatePeaks program provided by HOMER (Heinz et al., 2010).

(xv) ATAC-Sequencing Library Preparation and Data Processing

ATAC-seq was performed as previously described (Buenrostro et al., 2013). Briefly, 20,000 cells were collected for ATAC-seq library preparation at ctrl D10, miNs D10 and miNs D20. Transposition reaction was carried out with Nextera Tn5 Transposase for 30 min at 37° C. Library fragments were amplified for optimal amplification condition. Final libraries were purified using Ampure XP beads (Ampure) and sequenced with 50 bp paired-end reads on Illumina HiSeq 2500.

More than 50 million ATAC-seq reads were trimmed for Nextera adapter sequences using TrimGalore and aligned to hg19 human genome assembly using bowtie2 with parameters—very-sensitive—maxins 2000—no-discordant—no-mixed. Duplicate reads were discarded with Picard and uniquely mapped reads were used for downstream analysis. Peaks were called using Homer with parameters findPeaks-region-size 150-minDist 300. Peaks called from all the samples were combined together and raw reads mapped on the combined peaks were counted using HTSeq count. Differential peaks between any two different samples were identified using edgeR with a cut-off: a fold-change threshold of 1.5 and FDR<0.01. Differential peaks were regarded as peaks that are gained or lost at each time point.

Gained peaks at miNs D10 and D20 were combined together and defined as open chromatin regions. Conversely, all lost peaks at miNs D10 and D20 were defined as close chromatin regions. The genomic features in the differential open and close chromatin regions were distributed by the CEAS software (Shin et al., 2009). Ref-seq genes that are most nearest located from differential peaks with Homer annotatePeaks command were annotated. Based on those genomic distribution and peak annotation, the promoter regions (−/+2Kb of TSS) and distal regions (all peak positions except the promoter regions) were defined. GO enrichment analysis was performed by Metascape or the Gene Ontology. All heatmaps were generated based on normalized signal intensity values (i.e. log₂CPM) of each sample on relevant specific regions.

All histone mark ChIP-seq data were obtained from Roadmap Epigenome database of human fibroblasts (Roadmap Epigenomics et al., 2015). To identify histone mark-occupied chromatin accessibility during reprogramming, each histone ChIP-seq data was compared with open and close chromatin regions based on ATAC-seq. It was confirmed that most open and closed chromatin peaks overlapped with histone mark-ChIP peaks were found in regions outside of promoter regions (+/−2Kb of TSS). Open and closed chromatin regions excluding the promoter regions were then used to compare with histone ChIP-seq data and perform further GO enrichment analysis.

(xvi) Translating Ribosome Affinity Purification

Translating ribosome affinity purification (Heiman et al., 2014) was performed on spinal cord dissections pooled from 3-4 mice 21 days post birth that were positive for the eGFP-L10A fusion ribosomal marker protein under the expression of either the Chat promoter (Tg(Chat-EGFP/Rpl10a)DW167 Htz) or the Snap25 promoter (Tg(Snap25-EGFP/Rpl10a)JD362Jdd). TRAP samples underwent immunopurification for four hours at 4° C. Both TRAP and pre-immunopurification control RNA samples were extracted through TRIzol purification, DNase treatment, and Qiagen RNeasy Mini columns (74104). Quality and quantity of RNA was assessed using a Bioanalyzer 2100 RNA Pico Chip. Sequencing libraries were amplified using Nugen Amplification Kit Ovation@ RNA-Seq System V2 (7102). Genome Technology Access Center at Washington University in St. Louis performed adapter ligation and sequencing of the libraries on the Illumina Hiseq2500. Three replicates of this procedure were analyzed.

(xvii) Analysis of TRAP RNA-Seq Data

RNA-Seq reads were mapped to Ensembl release 76 using STAR (analysis performed by Genome Technology Access Center at Washington University in St. Louis). For downstream analyses, only those genes with >1 CPM in at least 3 samples, with an Ensembl gene biotype of “protein_coding,” were retained. For gene symbols mapping to multiple Ensembl gene IDs, only the ID with the highest number of mapped reads was retained, resulting in a total of 14,009 genes used for downstream analyses. Using edgeR, read counts were fit to a negative binomial generalized log-linear model, and a likelihood ratio test was done to determine differential expression.

(xviii) Comparative Analysis of RNA-Seq and Microarray Data

For comparative analysis, only probes with a detected call in at least 1 of 6 samples was retained, resulting in 23,775 probes mapping to a gene symbol. Expression level was then averaged over all probes for each gene, resulting in a total of 15,333 genes that were used for comparative analysis, 10,736 of which were also present in the gene set retained from the RNA-seq dataset (described above) after CPM filtering. Within the genes retained in both datasets, the top differentially expressed genes between motor neurons and controls-CHAT IP vs. SNAP25 IP (logFC>1 and p<0.05) in the RNA-seq dataset, and Moto-miN vs. miN (logFC>2.5 and p<0.05) in the microarray dataset-were assessed for significant contingency using a one-tailed Fisher's exact test.

(xix) Quantitative PCR

Total RNA was extracted using TRIzol (Invitrogen, USA) according to the manufacturer's instruction. Reverse-transcribed complementary DNA (cDNA) was synthesized from 500 ng of RNA with SuperScript III First-Strand Synthesis SuperMix (Invitrogen, USA) or from 10 ng of RNA for microRNAs expression analyses using specific stem-loop primer probes from TaqMan MicroRNA Assays (Invitrogen, USA). Subsequently, the cDNA was analyzed on a StepOnePlus Real-Time PCR System (AB Applied Biosystems, Germany). Expression data were normalized to housekeeping genes HPRT1 and RNU44 for coding genes and microRNAs, respectively, and analyzed using the 2-^{ΔΔCT} relative quantification method. The following primers were utilized:

TABLE 1
Primer sequences used for qRT
PCR analysis (related to STAR
Methods section).

| Primer | |
|---|---|
| Name | Sequence (5′-3′) |
| S100A4 FWD | GATGAGCAACTTGGACAGCAA |
| S100A4 REV | CTGGGCTGCTTATCTGGGAAG |
| VIM FWD | AGTCCACTGAGTACCGGAGAC |
| VIM REV | CATTTCACGCATCTGGCGTTC |
| COL13A1 | GGAGACGGCTATTTTGGGACG |
| FWD | |
| COL13A1 | TOCTTGAGIGGAGOTTCCATT |
| REV | |
| ChAT FWD | TCAATCATGTCCAGCGAGTC |
| ChAT REV | AACGAGGACGAGCGTTTG |
| HB9 FWD | CTCCTACTCGTACCCGCAG |
| HB9 REV | TTGAAGTCGGGCATCTTAGGC |
| SLC18A3 | TTCGCCTOTACAGTOCTGTTC |
| FWD | |
| SLC18A3 | GCTOCTCOGGGTACTTATCG |
| REV | |
| HOXB4 FWD | CGTGAGCACGGTAAACCCC |
| HOXB4 REV | CGAGCGGATCTTGGTGTTG |
| HOXC6 FWD | ACAGACCTCAATCGCTCAGGA |
| HOXC6 REV | AGGGGTAAATCTGGATACTGGC |
| HOXA7 FWD | CGTTCCGGGCTTATACAATGT |
| HOXA7 REV | CTCGTCCGTCTTGTCGCAG |
| HOXB7 FWD | TTCCCAGAACAAACTTCTTGTGC |
| HOXB7 REV | GCATGTTGAAGGAACTCGGCT |
| HOXC8 FWD | ACCGGCCTATTACGACTGC |
| HOXC8 REV | TGCTGGTAGCCTGAGTTGGA |
| HOXD8 FWD | GGAAGACAAACCTACAGTCGC |
| HOXD8 REV | TCCTGGTCAGATAGGGGTTAAAA |
| HOXA9 FWD | TACGTGGACTCGTTCCTGCT |
| HOXA9 REV | CGTCGCCTTGGACTGGAAG |
| HOXC9 FWD | ACTCGCTCATCTCTCACGACA |
| HOXC9 REV | GACGGAAAATCGCTACAGTCC |
| HOXD9 FWD | GGACTCGCTTATAGGCCATGA |
| HOXD9 REV | GCAAAACTACACGAGGCGAA |
| HOXC10 | ACATGCCCTCGCAATGTAACT |
| FWD | |
| HOXC10 | GAGAGGTAGGACGGATAGGTG |
| REV | |
| HOXC11 | ATGTTTAACTCGGTCAACCTGG |
| FWD | |
| HOXC11 | GCATGTAGTAAGTGCAACTGGG |
| REV | |
| HOXD11 | TCTCCGAGTCCTCGTGGGGA |
| FWD | |
| HOXD11 | GCAAAACACCAGCGCCTTCTA |
| REV | |
| HPRT FWD | TCCTTGGTCAGGCAGTATAATCC |
| HPRT REV | GTCAAGGGCATATCCTACAACAAA |

miRNA oRT-PCR primers.
hsa-miR-218 (Thermo Fisher Cat. #4427975)
RNU-44 (Thermo Fisher Cat. #4427975)
Overlap with ISL1/LHX3 ChlP Seg,

To identify the genes regulated by LHX3 and ISL in motor neurons, ISL- and LHX3-ChIP sequencing data (Mazzoni et al., 2013) were used. The regions co-occupied by ISL and LHX3 were selected during ES to motor neuron differentiation, accounting for 84.2% of peak regions called in each ChIP-seq data. Based on the peaks co-occupied by ISL and LHX3, 3,486 closest genes with peaks located within 5Kb upstream of TSS and intragenic regions were annotated. Comparing those annotated genes with genes selectively enriched in Moto-miNs vs miNs (log₂ fold change=>2.5, p<0.01), identified 323 genes co-occupied by ISL1 and LHX3 that are also upregulated when miR-9/9*-124 is co-expressed with ISL1/LHX3.

### Example 2: Modeling Huntington's Disease (Hd) Wt Neurons Directly Converted from Patient Faroblasts

The following example describes the generation of medium spiny neurons (MSNs) from Huntington's Disease (HD) patient fibroblasts through microRNA-based neuronal conversion.

Generation of MSNs from HD Patient Fibroblasts

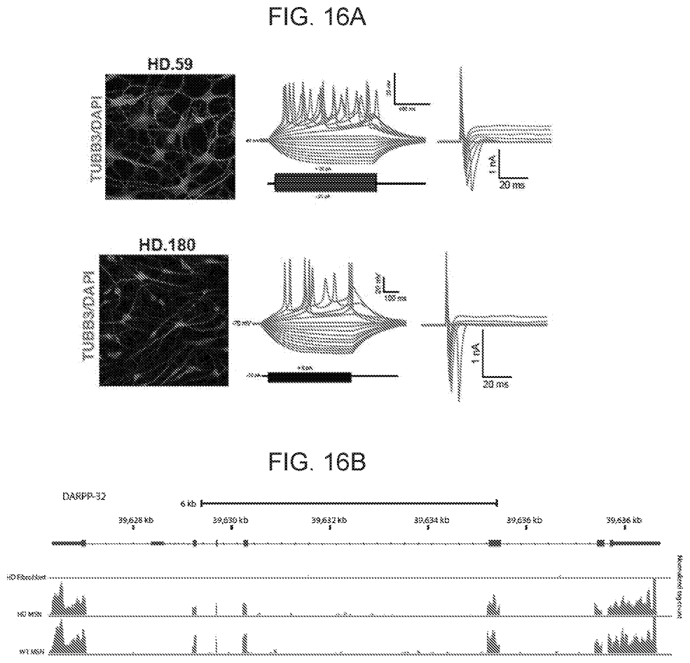

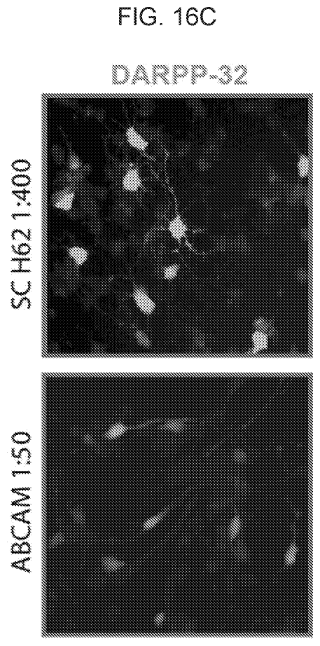

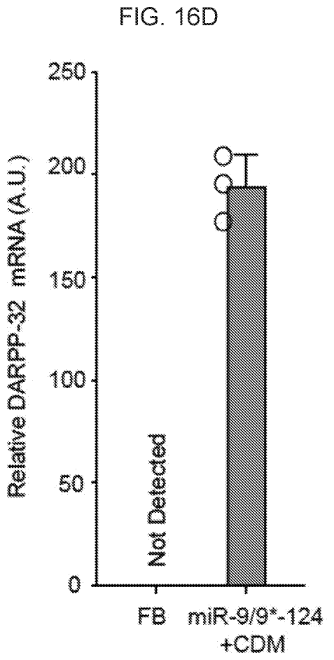

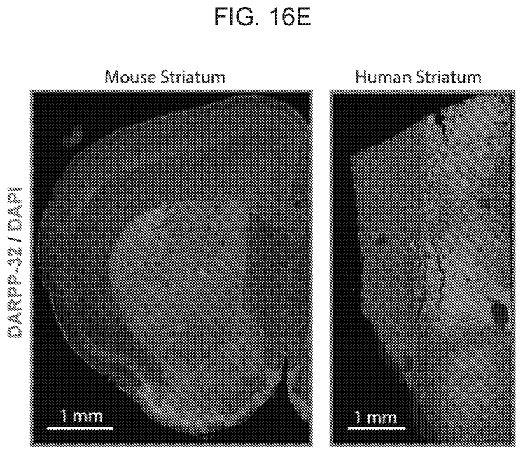

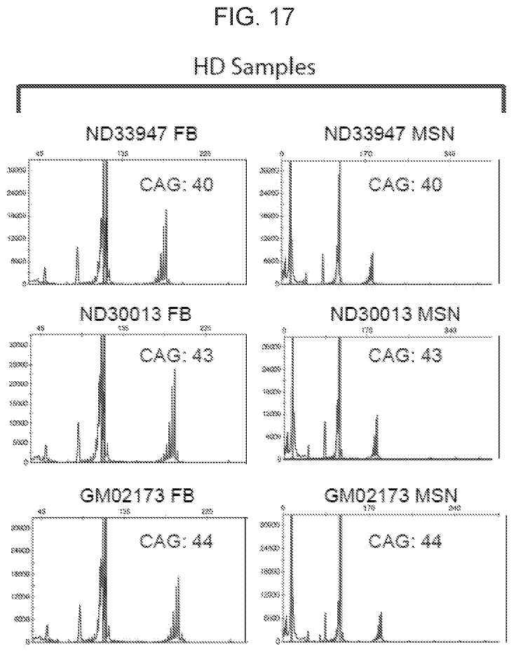

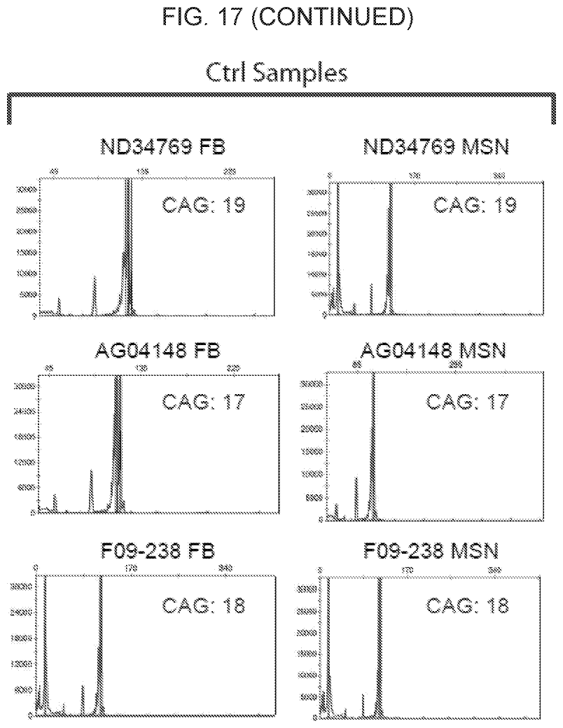

The efficacy of miR-9/9*-124+CDM-based neuronal conversion in HD patient samples was first tested. Fibroblasts were obtained from ten symptomatic HD patients including both males and females, ranging from 6 to 71 years of age with various CAG-repeat expansions (40 to 180) in HTT (see e.g., TABLE 2). It was found that HD fibroblasts could be directly reprogrammed to MSNs regardless of age or number of CAG repeats (see e.g., FIG. 15 and FIG. 16). As such, the present analysis was elected to focus the on patient samples with pathologic CAG repeats lower than 50 because this range represents the majority of adult-onset cases that remain understudied. MSN conversion was validated using three independent HD patient fibroblast samples containing 40, 43, or 44 CAG repeats (HD.40, HD.43 and HD.44) and their respective age- and gender-matched healthy controls (Ctrls) with 19, 17, or 18 CAG repeats (Ctrl. 19, Ctrl. 17, and Ctrl. 18) (see e.g., FIG. 15). When analyzed at post-induction day 30 (PID 30), HD-MSNs expressed the neuronal markers TUBB3, NeuN and MAP2, GABAergic neuron marker GABA, and the MSN marker and DARPP-32 (see e.g., FIG. 15A and FIG. 16). No significant differences were found in the reprogramming efficiency between HD and control samples, with both groups generating approximately 90% MAP2-positive cells, 70-80% GABA-positive cells, and 70-80% DARPP-32-positive neurons (see e.g., FIG. 15B for representative immunostaining; quantification in FIG. 15C). Furthermore, CAG repeat lengths remained stable after neuronal conversion (see e.g., FIG. 17).

TABLE 2

| | Line (Coriell | | Age at | | | | Upper |
|---|---|---|---|---|---|---|---|
| Sample ID | Biorepository) | Sex | Sampling | Genotype | Diseases | Onset | CAG |
| HD.44 | GM02173 | Female | 52 | HD | Symptomatic | NR | 44 |
| HD.45 | GM04230 | Male | 55 | HD | Symptomatic | NR | 45 |
| HD.46 | GM04194 | Female | 60 | HD | Symptomatic | NR | 46 |
| HD.47 | GM04198 | Female | 63 | HD | Symptomatic | NR | 47 |
| HD.42 | GM04196 | Female | 51 | HD | Symptomatic | NR | 42 |
| HD.40 | ND33947 | Female | 71 | HD | Symptomatic | 63 | 40 |
| HD.43 | ND30013 | Male | 54 | HD | Symptomatic | 50 | 43 |
| HD.1 | GM02147 | Male | 55 | HD | Symptomatic | NR | NR |
| HD.180 | GM09192 | Male | 6 | HD | Symptomatic | <6 | 180 |
| HD.50 | GM04682 | Female | 37 | HD | Symptomatic | 27 | 50 |
| Pre-HD.42 | GM04717 | Female | 44 | HD | Pre-symptomatic | 60 | 42 |
| Pre-HD.45 | [symbol] | Male | 16 | HD | Pre-symptomatic | [symbol] | 45 |
| Pre-HD.47 | [symbol] | Male | 11 | HD | Pre-symptomatic | 26 | 47 |
| Pre-HD.43 | [symbol] | Male | 23 | HD | Pre-symptomatic | 40 | 43 |
| Pre-HD.49 | [symbol] | Male | 15 | HD | Pre-symptomatic | 30 | 49 |
| Pre-HD.47b | [symbol] | Female | 21 | HD | Pre-symptomatic | 34 | 47 |
| Ctrl.17 | [symbol] | Male | 56 | Control | Healthy | N/A | 17 |
| Ctrl.17b | GM02171 | Female | 72 | Control | Healthy | N/A | 17 |
| Ctrl.18 | [symbol] | Female | 42 | Control | Healthy | N/A | 19 |
| Ctrl.19 | [symbol] | Female | [symbol] | Control | Healthy | N/A | 19 |
| Ctrl.16 | AG11357 | Female | [symbol] | Control | Healthy | N/A | 16 |
| Ctrl.15 | AG11483 | Female | 51 | Control | Healthy | N/A | 15 |
| Ctrl.1 | GM05879 | Female | 48 | Control | Healthy | N/A | NR |
| Ctrl.2 | AG16409 | Male | 17 | Control | Healthy | N/A | NR |
| Ctrl.3 | AG08816 | Male | 39 | Control | Healthy | N/A | NR |
| Ctrl.4 | [symbol] | Female | 30 | Control | Healthy | N/A | NR |
| Ctrl.20 | [symbol] | Female | 51 | Control | Healthy | N/A | 20 |
| Ctrl.18b | AG04062 | Male | 31 | Control | Healthy | N/A | 18 |
| Ctrl.17c | [symbol] | Male | 44 | Control | Healthy | N/A | 17 |
| HD.50IPSC | ND42235 | Derived | | | | | |
| | | from HD.50 | | | | | |
| HD.40IPSC | ND33942 | Derived | | | | | |
| | | from HD.40 | | | | | |
| | | in house | | | | | |

[symbol]

indicates data missing or illegible when filed

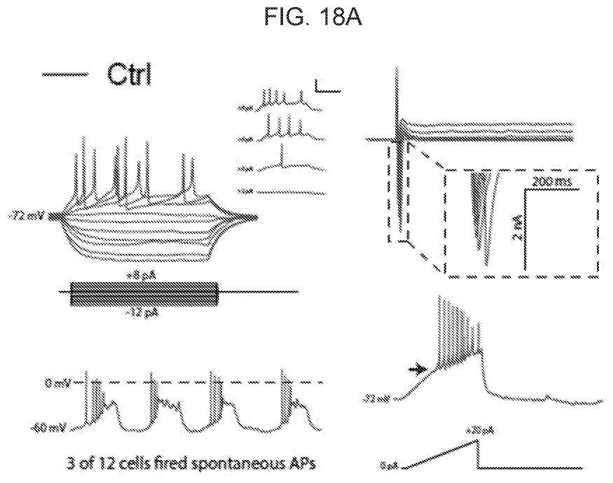

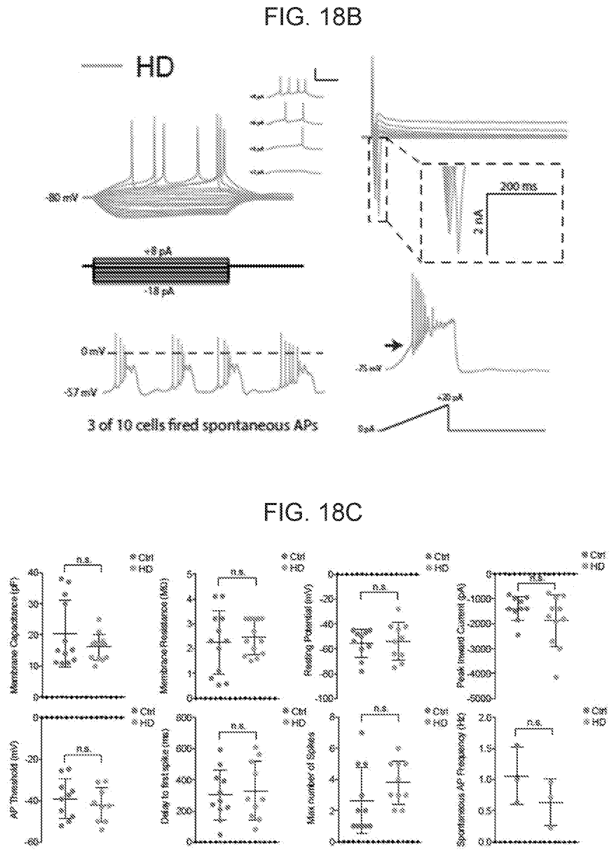

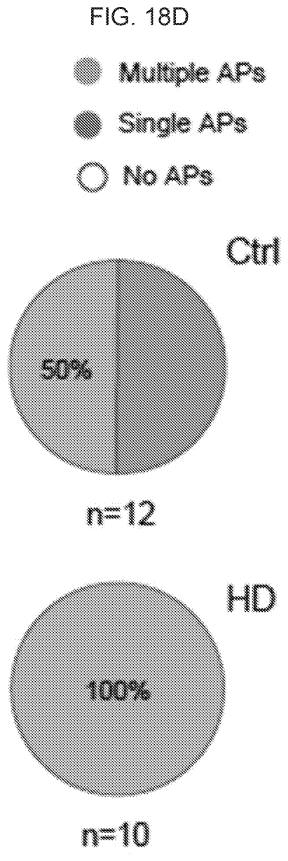

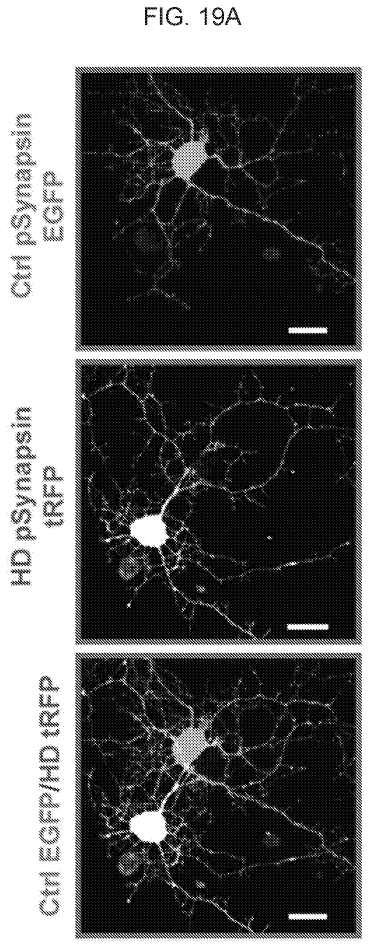

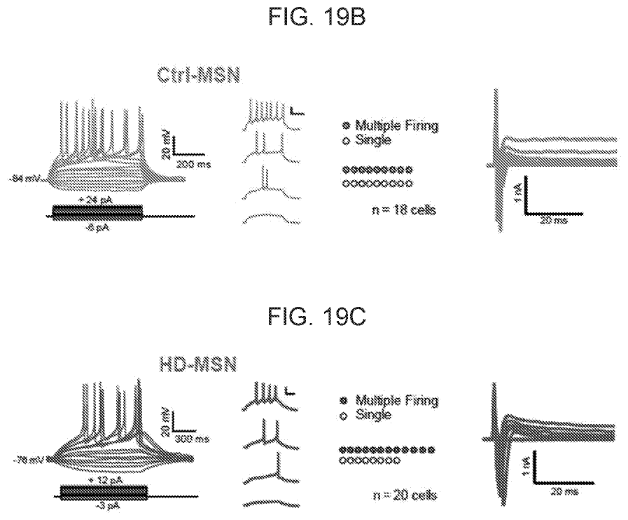

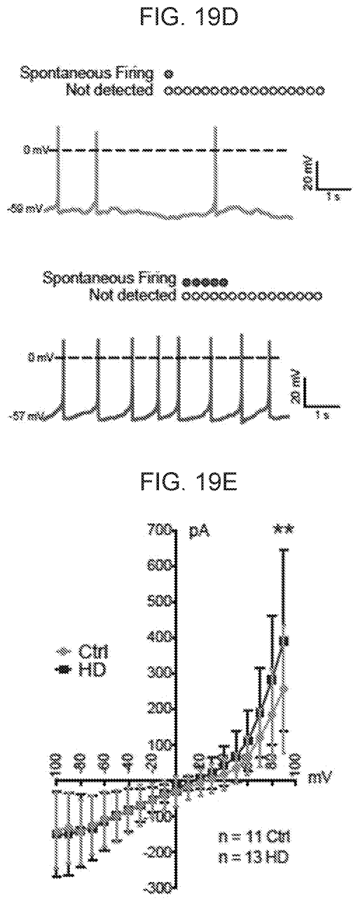

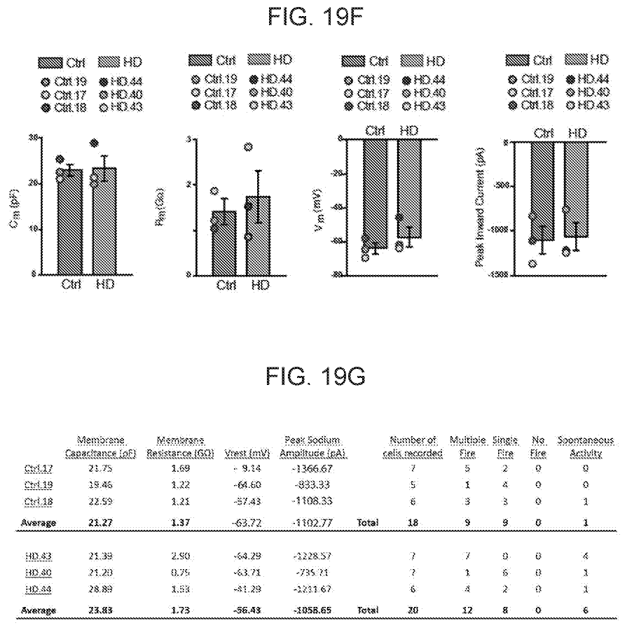

Electrophysiological studies in mouse models of HD have collectively demonstrated alterations in synaptic properties of striatal MSNs at different stages of disease progression. In order to determine potential functional differences, the properties of HD.47-MSNs and Ctrl. 16-MSNs were assessed by whole-cell recording. All recorded cells displayed neuronal properties, including multiple action potentials and robust inward and outward currents upon stimulation (see e.g., FIG. 18A). Both HD-MSNs and Ctrl-MSNs displayed spontaneous action potentials at similar frequencies, and action potential thresholds that did not differ significantly (see e.g., FIG. 18A-FIG. 18C). Notably, a greater number of neurons fired multiple action potentials in HD-MSNs than in Ctrt-MSNs (see e.g., FIG. 18D). All other passive membrane properties recorded, however, did not differ significantly between HD- and Ctrl-MSNs (see e.g., FIG. 18C). To further access electrophysiological properties under the same recording condition, HD-MSNs (HD.40, HD.43 and HD.44) and Ctrl-MSNs (Ctrl. 17, Crtl. 18, and Crtl. 19) were co-cultured tobe recorded on the same coverslip (see e.g., FIG. 19). At PD35, all six reprogrammed lines fired action potentials, but consistent with cells separately cultured, a greater number of HO-MSNs fired multiple action potentials than Ctrl-MSNs upon current injections (see e.g., FIG. 19B-FIG. 19C) and displayed spontaneous generation of action potentials (see e.g., FIG. 19D). Whereas passive membrane properties measured remained similar between HD- and Ctrl-MSNs, increased current responses with high voltage stimulus were detected in HD-MSNs (see e.g., FIG. 19E-FIG. 19F). This increase in firing complexity and excitability of HD-MSNs, is consistent with the increase in frequency and amplitude of spontaneous postsynaptic currents previously reported in R6/2 and YAC128 mouse models of HD.

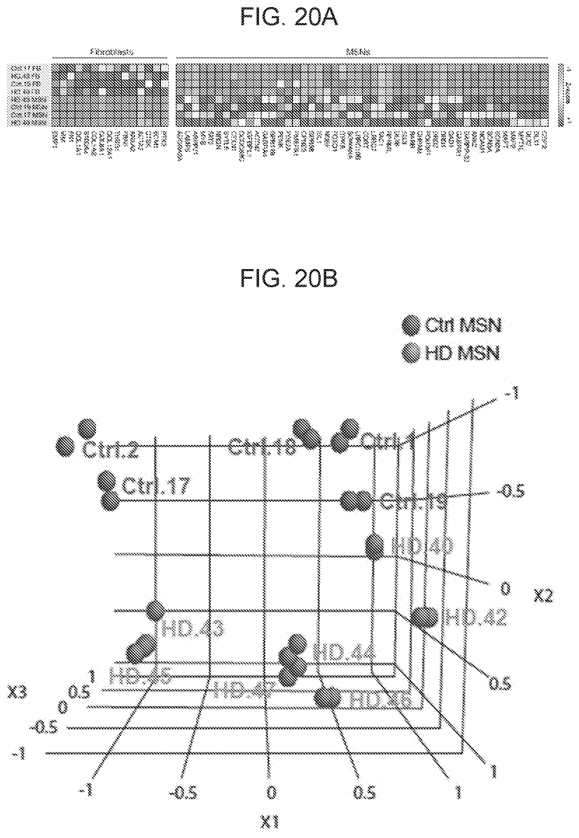

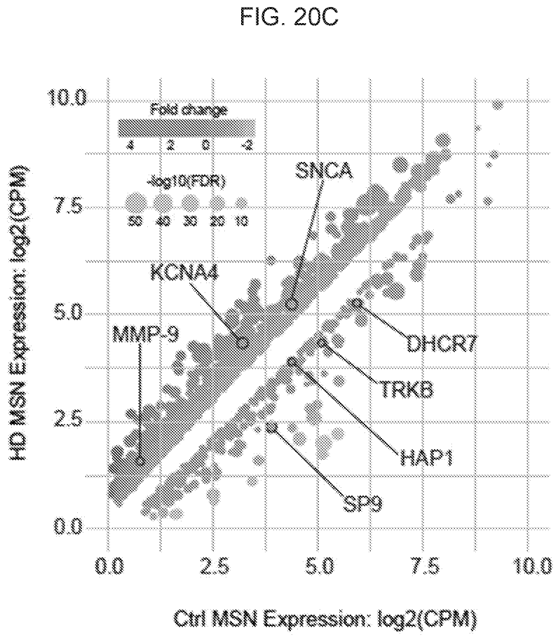

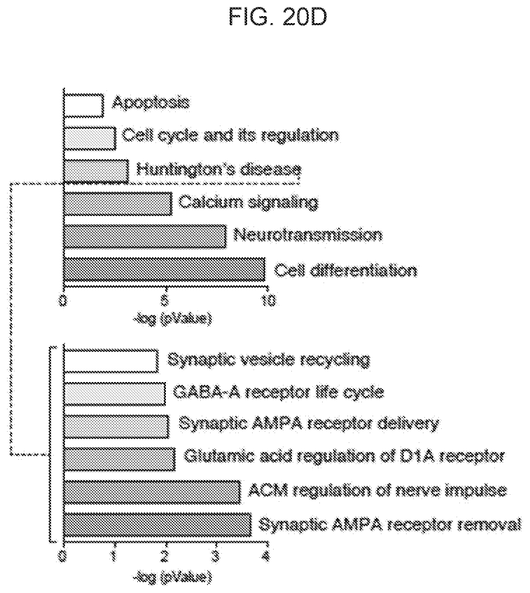

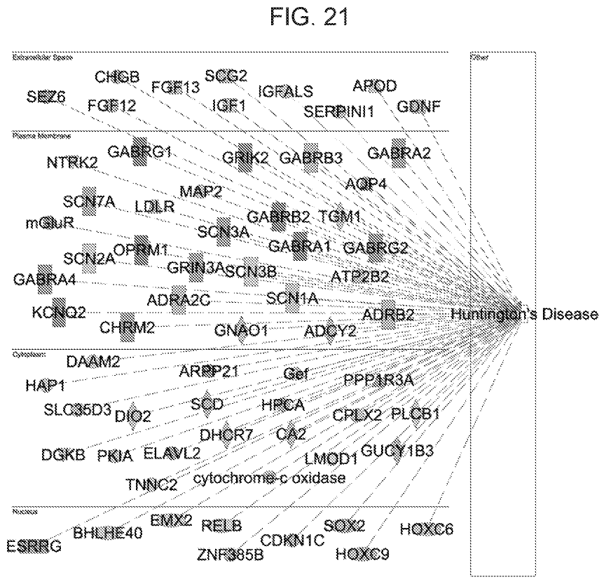

To further analyze the proper acquisition of striatal fate, RNA sequencing (RNA-seq) analysis was first performed at PID 32 and compared the gene expression profile between fibroblasts and converted neurons in HD and control samples. Analysis of 15 representative fibroblast-associated genes and of 53 genes highly enriched in the striatum revealed the successful acquisition of MSN fate in neurons converted from HD and control samples (see e.g., FIG. 20A). In order to identify genes that might be dysregulated in HD-MSNs, transcriptional analysis of 7 independent HD-MSN and 5 Ctrl-MSN samples was carried out (TABLE 2). Principal component analysis indicated separation of samples mainly based on the genotype (mHTT vs healthy control) as well as the gender of sample donors (see e.g., FIG. 20B). DEG analysis of protein-coding genes revealed 1,127 differentially expressed genes (DEGs) in HD-MSNs (false-discovery rate (FDR) 50.01 and log fold-change (LFC)≥0.5) (see e.g., FIG. 20C). Through gene ontology analysis, DEGs in HD-MSNs were found to be significantly enriched for genetic networks associated with cell differentiation (p-value of 1.51×10⁻¹⁰), neurotransmission (p-value of 1.31×10⁻⁶), calcium signaling (p-value of 5.31×10⁻⁶), HD (p-value of 7.22×10⁻⁴), and apoptosis (p-value of 1.19×10²) (see e.g., FIG. 20D and corresponding genes listed in TABLE 3). Several of the DEGs identified in the present experiment have been previously implicated in HD. For example, the upregulation of the matrix metalloproteinase 9 (MMP-9) was detected which has been shown to be increased in postmortem human HD brains and to significantly decrease the survival of striatal neurons. Moreover, the analysis revealed the downregulation of Huntington-associated protein-1 (HAP1) (LFC−0.55 and FDR 5.04×10⁻⁴) (see e.g., FIG. 20C), which has been previously shown to antagonize mHTT-mediated cytotoxicity and enhance cell viability. Additionally, there was downregulation of 7-dehydrocholesterol reductase (DHCR7) (LFC−0.71 and FDR 2.8×10⁻⁵) (see e.g., FIG. 20C), an enzyme previously shown to have reduced expression in patients and mouse models of HD, and thought to be involved in HD-specific metabolic pathway alterations. Notably, the majority of HD-related DEGs are upregulated in HD-MSNs and are involved in neurophysiological processes, such as the voltage-gated potassium channel subunit KCNA4 (LFC 1.15 and FDR 1.1×10⁴) (see e.g., FIG. 20C), in addition to several subunits of GABA type-A receptors and AMPA receptors, suggesting increased neurotransmission in HD-MSNs (see e.g., FIG. 20C-FIG. 20D and FIG. 21). We also detected upregulation of α-Synuclein (SNCA) (LFC 1.15 and FDR 1.1×10⁴), an aggregation-prone protein shown to accumulate in HD polyglutamine inclusions. Interestingly, overexpression of α-Synuclein has been reported to accelerate the onset of HD symptoms in multiple mouse models. Further, NTRK2 (also known as TRKB), the main receptor for the brain-derived neurotrophic factor (BDNF), was found to be downregulated in HD-MSNs (LFC-0.77 and FDR 7.44×10⁻⁴) (see e.g., FIG. 20C). The loss of BDNF in HD pathology has been investigated extensively and proposed to play a critical role in the degeneration of MSNs, and the results indicate that mHTT may induce a cell-intrinsic downregulation at the receptor level in HD-MSNs. Recently the impairment of TRKB receptor rather than the decreased level of BDNF was suggested to mediate postsynaptic dysfunction of MSNs in mouse models of HD, although changes in TRKB mRNA levels were not detected in HD mouse models⁴⁰. The analysis also uncovered DEGs with no previous association with HD, which is a promising area of future studies. For instance, the transcription factor SP9, which has been recently shown to be necessary for the survival of striatopallidal MSNs, is significantly downregulated (LFC-1.7 and FDR 1.49×10⁻⁸) in HD-MSNs. The transcriptome data implicates processes known to be affected in HD, reveals genes previously shown to be functionally important in disease onset and progression, and also identifies novel genes that warrant further investigation.

TABLE 3
Analysis of 7 Symptomatic HD and 5 healthy control MSNs
FDR 0.01 Log Fold---change cutoff 0.5.

| Analysis of 7 | | | |
|---|---|---|---|
| Symptomatic HD | | | |
| and 5 healthy control | | | |
| MSNs FDR 0.01 Log | | | |
| Fold-change | | | |
| cutoff 0.5 | Network Objects | P-value | FDR |
| Cell differentiation | [symbol] | [symbol] | [symbol] |
| Neurotransmission | [symbol] | [symbol] | [symbol] |
| Calcium signalling | [symbol] | [symbol] | [symbol] |
| Huntington Disease | [symbol] | [symbol] | [symbol] |
| Cell cycle and its regulation | [symbol] | [symbol] | [symbol] |
| Apoptosis | [symbol] | [symbol] | [symbol] |

[symbol]

indicates data missing or illegible when filed

Mutant HTT Aggregates in MSNs Directly Converted from HD Fibroblasts

Although a two-fold upregulation of HTT mRNA levels was observed in MSNs in comparison to starting fibroblasts, there are no significant changes in HTT mRNA levels in the analysis of HD-MSNs versus Ctrl-MSNs. This finding is not surprising given that HTT expression levels are comparable in the brains of HD and healthy patients. However, at the protein level, polyglutamine expansion within HTT leads to the formation of insoluble structures of aggregated mHTT, or inclusion bodies (IBs). The formation of mHTT inclusions in directly reprogrammed MSNs was then assessed by immunocytochemistry, ultrastructural, and biochemical analysis.

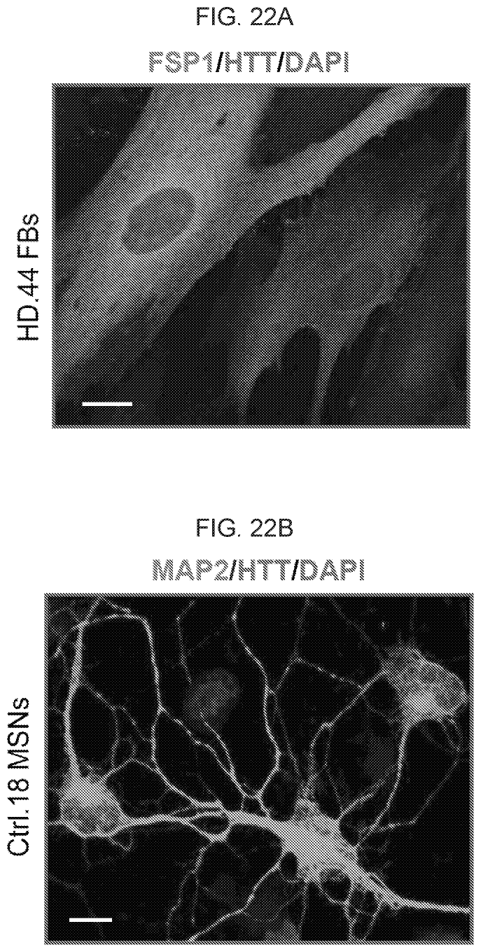

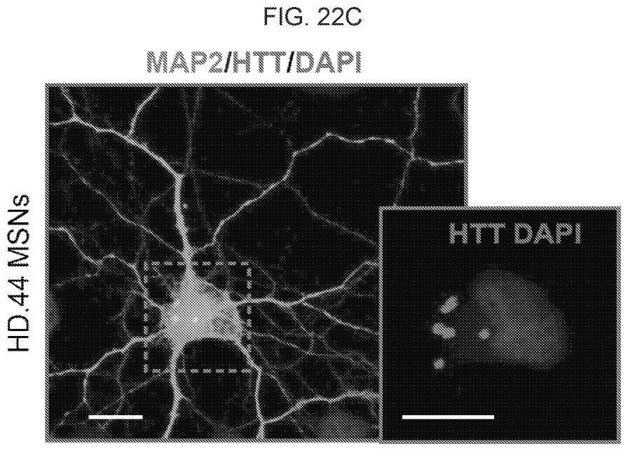

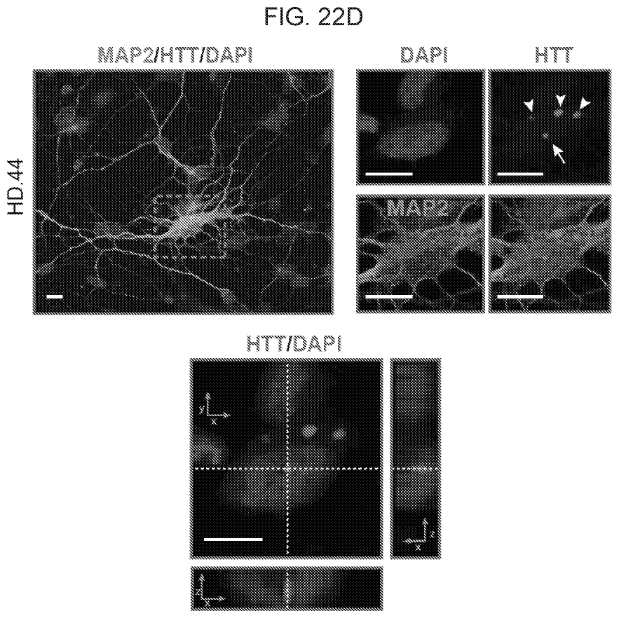

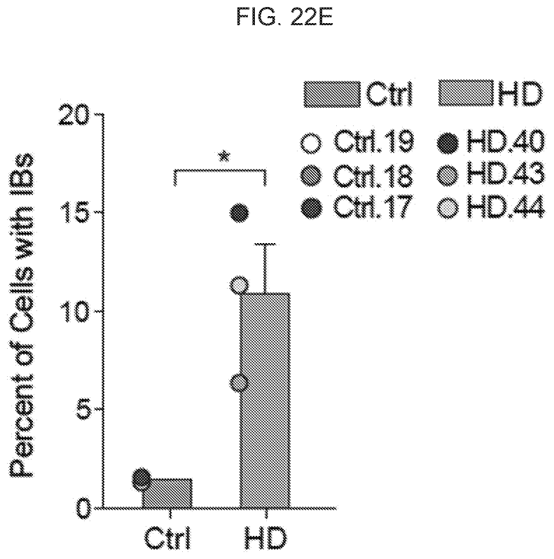

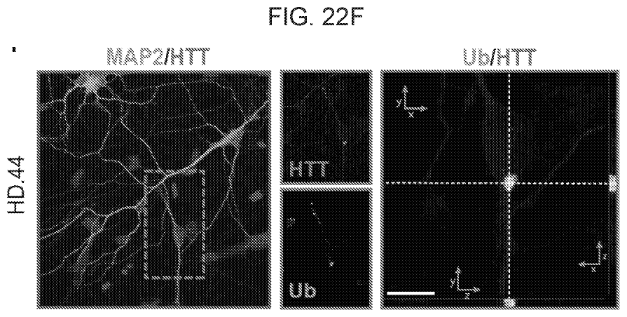

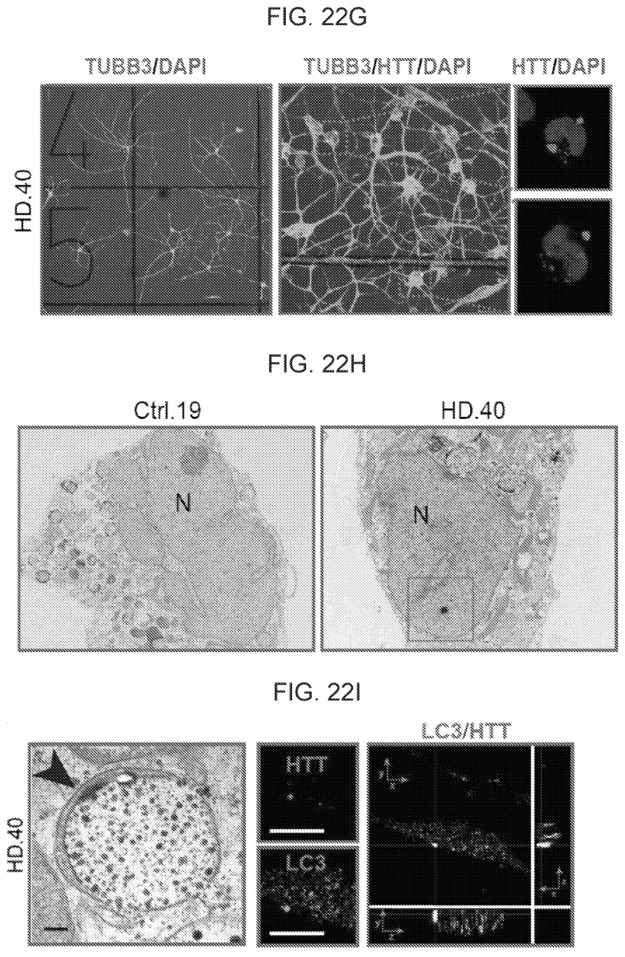

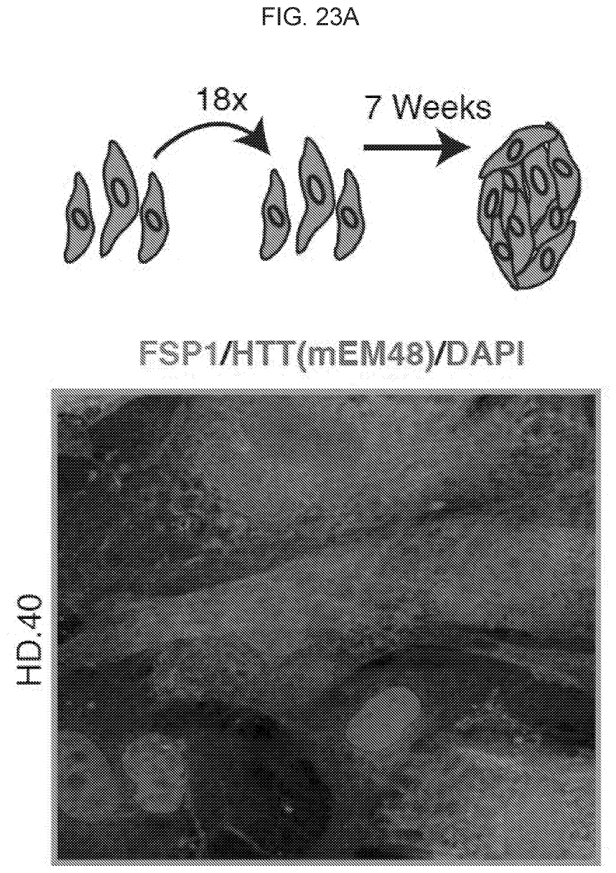

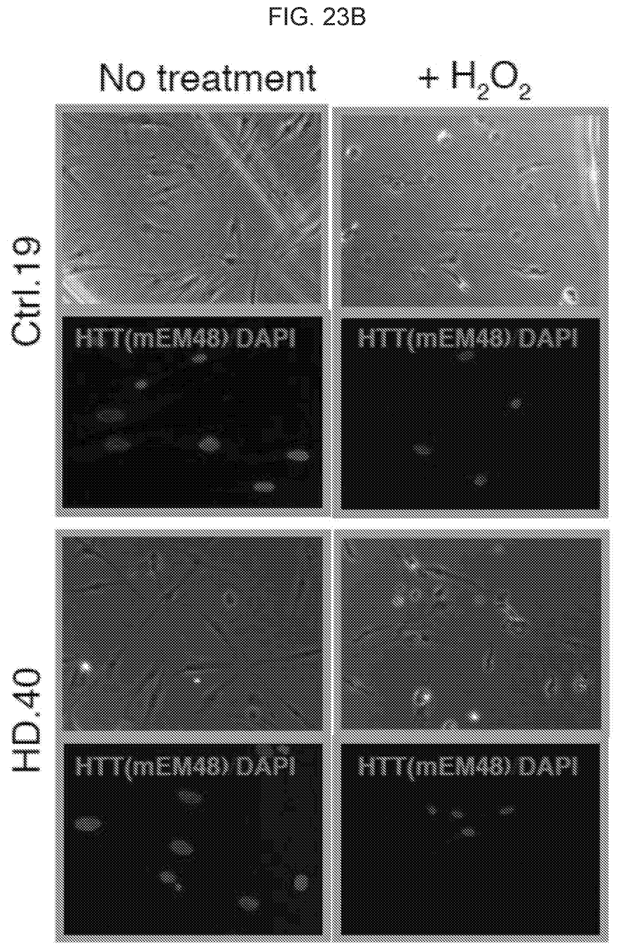

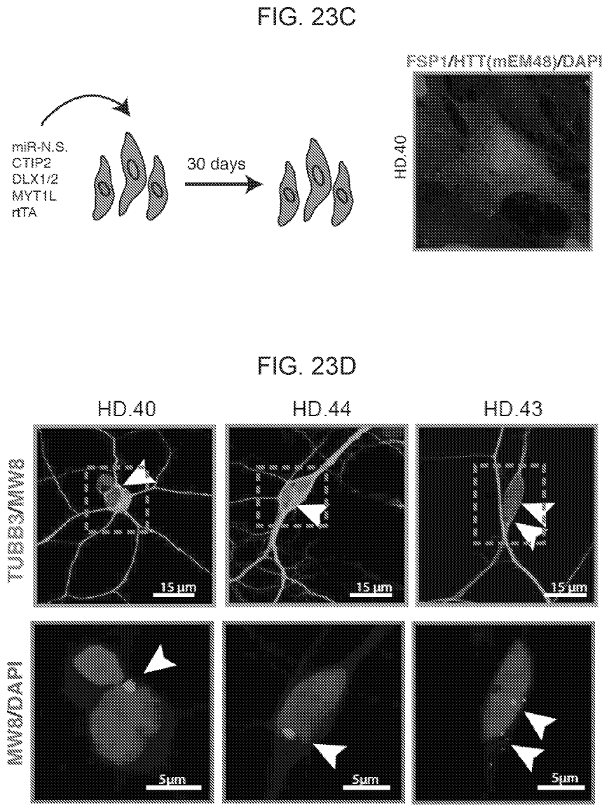

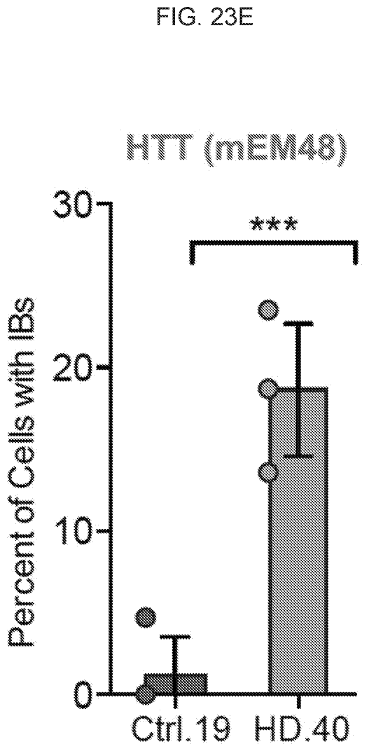

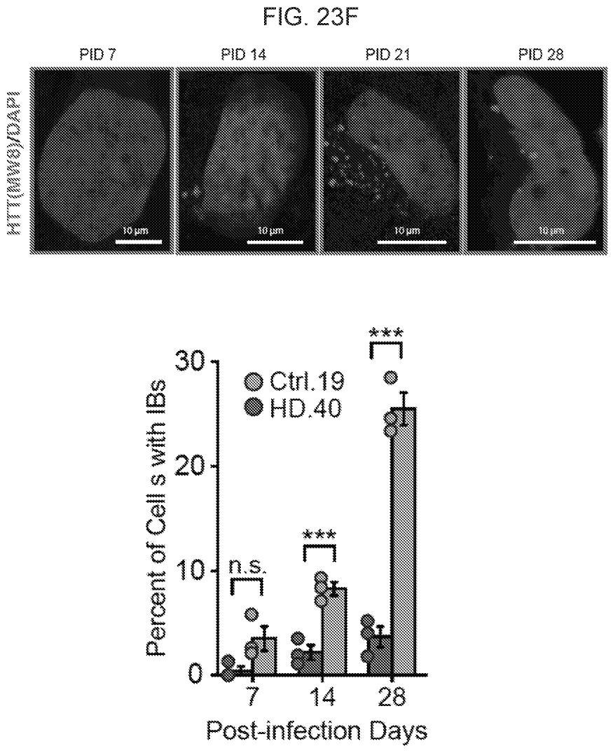

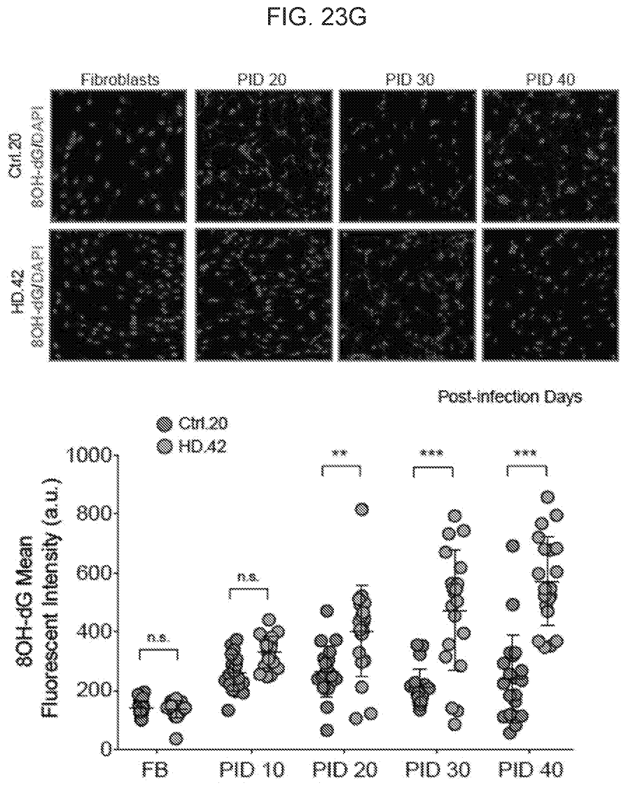

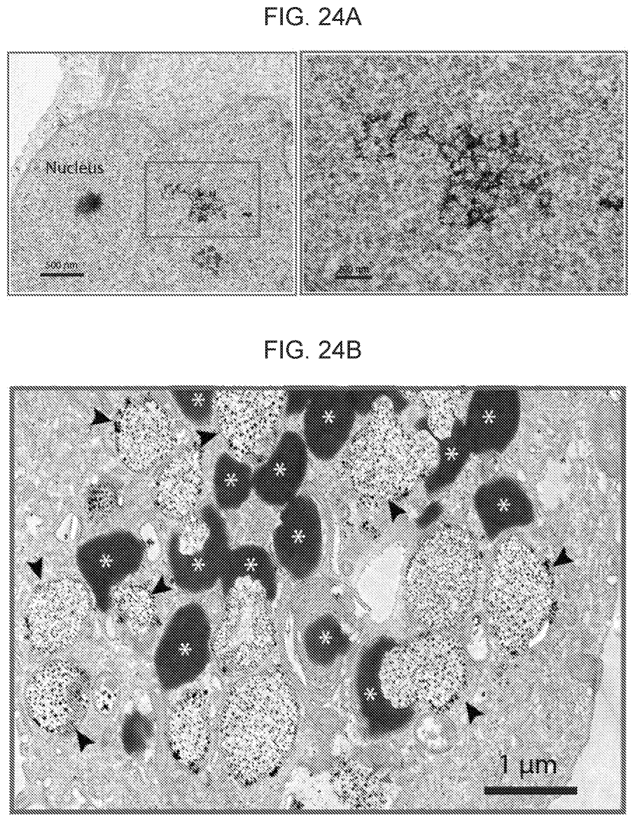

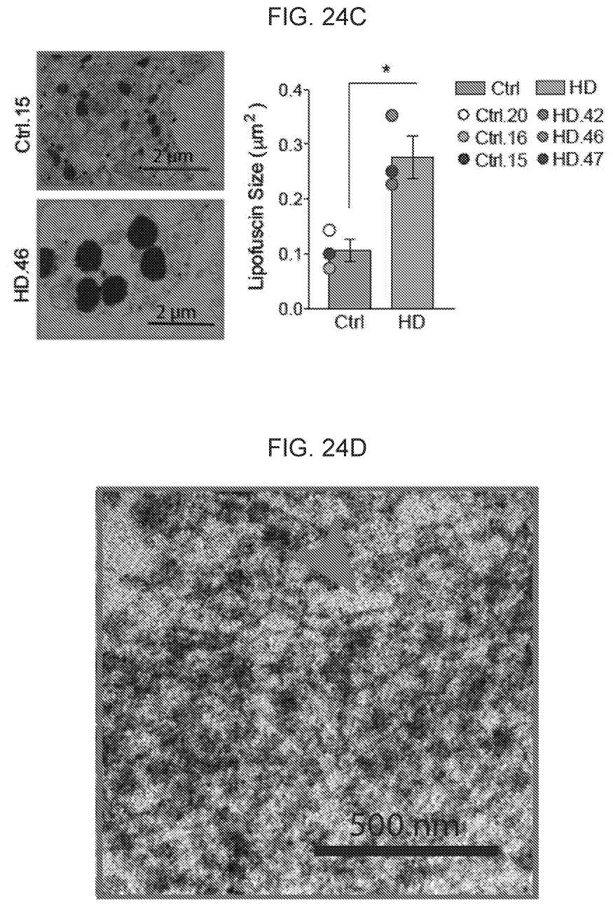

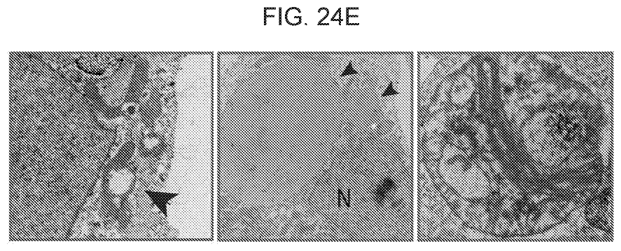

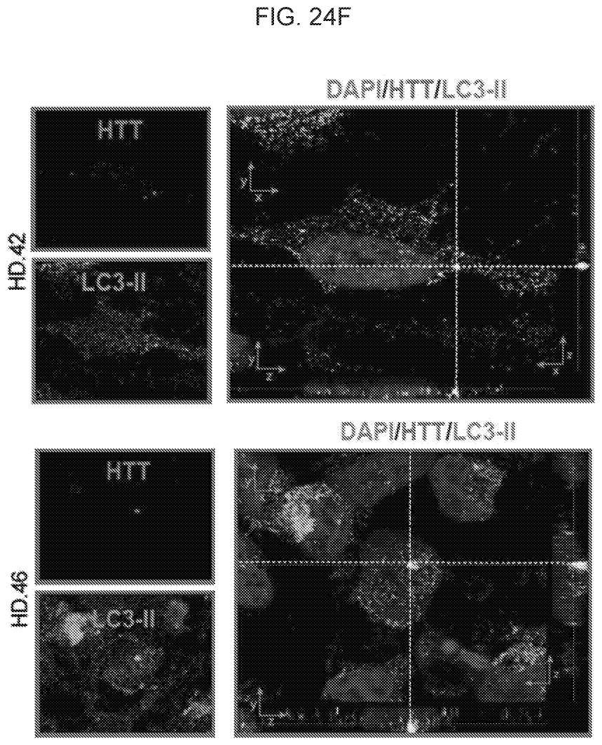

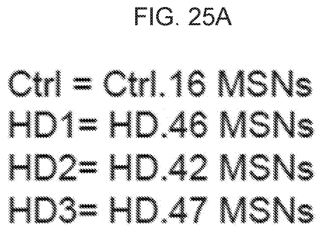

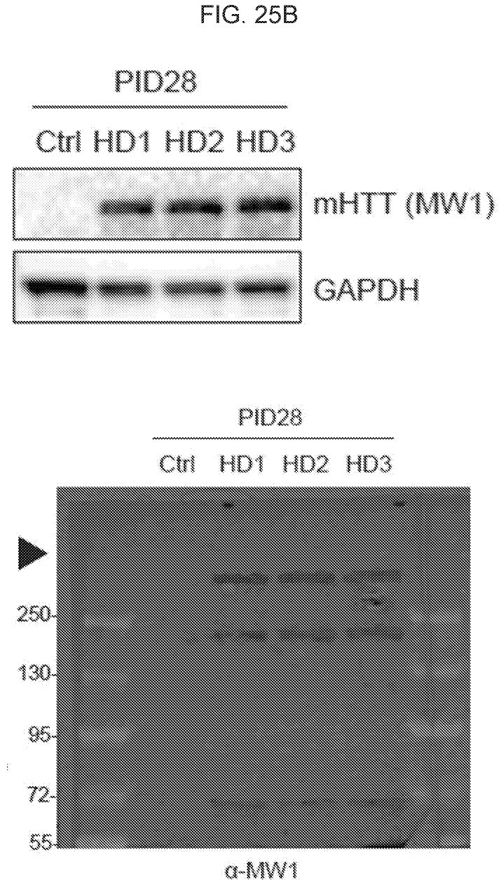

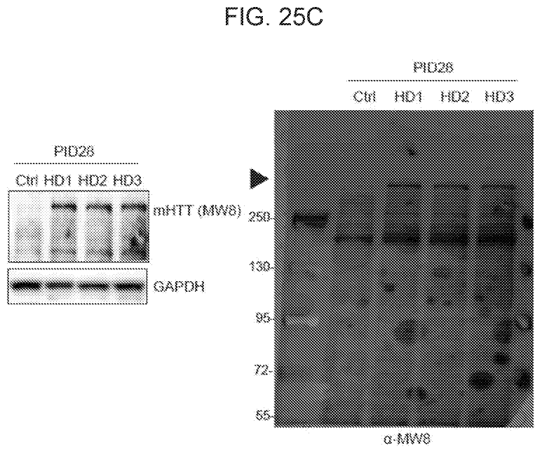

Noticeably, HD-MSNs exhibited mHTT aggregates in contrast to their corresponding fibroblasts or Ctrl-MSNs (see e.g., FIG. 22A, FIG. 22B and FIG. 22C). Non-reprogrammed HD fibroblasts were devoid of detectable mHTT aggregates even upon cellular insults, including the induction of oxidative stress with hydrogen peroxide or cellular senescence by serial passaging (see e.g., FIG. 23A and FIG. 23B). Furthermore, mimicking reprogramming using CDM factors with a non-specific microRNA, a condition previously shown to be ineffective for neuronal conversion, did not lead to detectable HTT aggregates (see e.g., FIG. 23C), demonstrating the specificity of the aggregation phenotype to successfully reprogrammed neurons. Cytoplasmic (see e.g., FIG. 22C and FIG. 22D—Arrowheads) and intranuclear (see e.g., FIG. 22D—Arrow) mHTT aggregates were evident in HD-MSNs reprogrammed from all HD patient samples as early as PID 14 when analyzed with distinctive antibodies (MW8 and EM48) that selectively recognize aggregated mHTT inclusion bodies, (IBs) that co-localized with ubiquitin (see e.g., FIG. 22F and FIG. 23D, FIG. 23E and FIG. 23F). HD models that have been engineered to overexpress mHTT with a large number of CAG repeats, report high levels of cells with inclusions. However, studies analyzing postmortem HD patient brains found that only up to 10% of MSNs had IBs, a number similar to the levels detected in HD-MSNs (see e.g., FIG. 23E). Examining the ultrastructure of immunogold-labeled mHTT inclusions by transmission electron microscopy in converted MSNs (HD.40 and Ctrl. 19) plated in micro-dishes (see e.g., FIG. 23G) revealed the presence of nanogold particles labeling mHTT aggregates within the nucleus, as well as structures of fibrillar morphology found only in HD-MSNs (see e.g., FIG. 22H and FIG. 24A). Expression of mHTT was further confirmed by immunoblot analysis at PID 28 in three HD-MSN samples (HD.42, HD.46 and HD.47) (see e.g., FIG. 25). The expression of soluble polyglutamine-expanded HTT was validated in MSNs reprogrammed from these three HD lines with the monoclonal antibody MW1, which has been previously shown to specifically detect the polyglutamine domain of HTT exon 1 while showing no detectable binding to normal HTT. Additionally, insoluble aggregated HTT can be detected biochemically in these three reprogrammed HD samples, but not in Ctrl-MSNs (Crtl. 16 MSNs) using the HTT aggregate-specific monoclonal antibody MW8 (see e.g., FIG. 25). Interestingly, via TEM studies, a large amount of immunogold particles were found compartmentalized inside autophagosomes, cytosolic double-membrane vesicles involved in macroautophagy (see e.g., FIG. 24B). This suggests that autophagic vacuoles can recognize and trap cytosolic mHTT inclusions in HD-MSNs harboring low CAG repeats in contrast to previously reported cargo recognition failure detected in the ¹¹¹Q-HTT HD mouse model. In fact, colocalization of mHTT and LC3-II, a well-established marker of autophagosomes, was observed in HD-MSNs reprogrammed from three independent HD lines, which is similar to previously reported findings in a HD mouse model (see e.g., FIG. 22I and Supplementary FIG. 24F).

Induction of Pluripotency Alters mHTT Aggregation Propensity

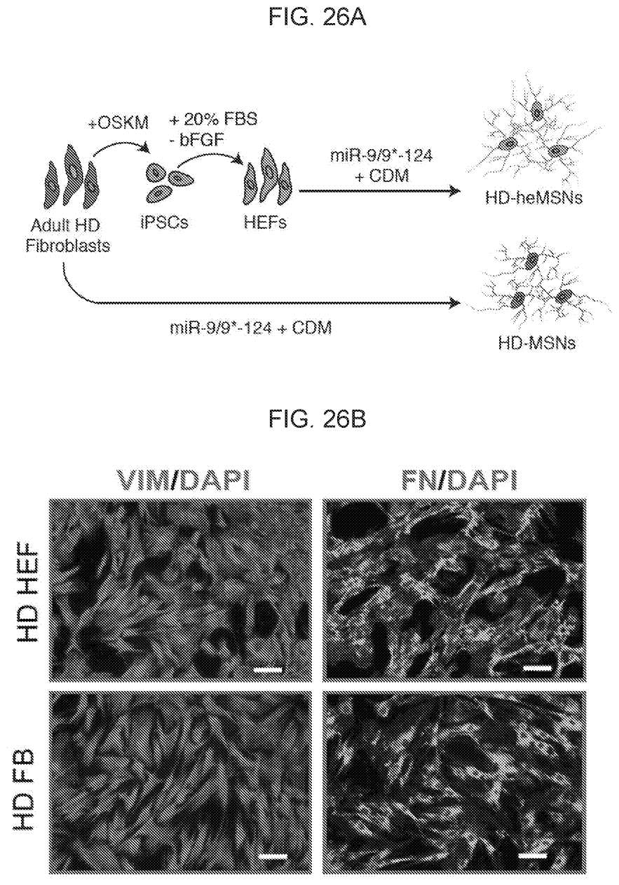

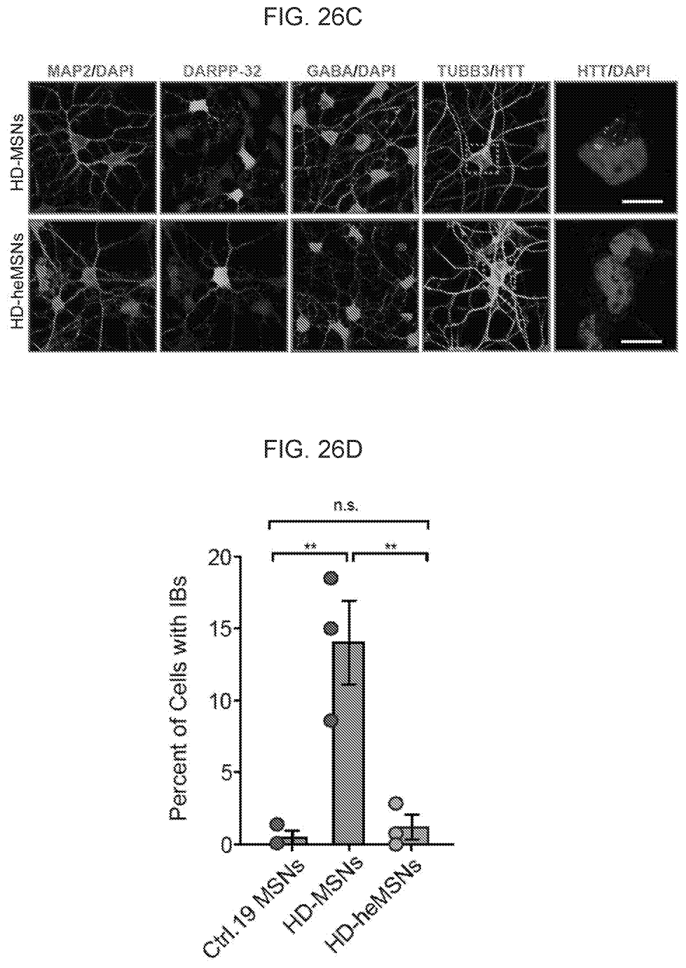

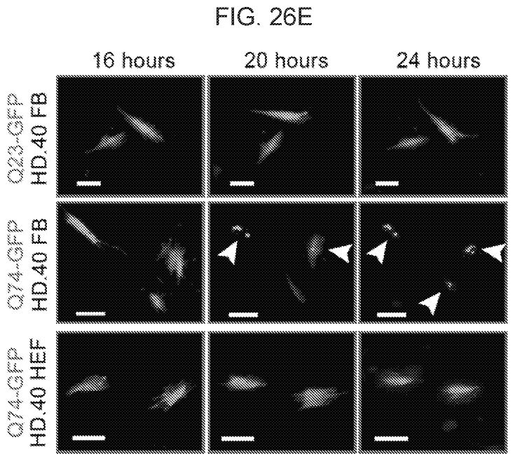

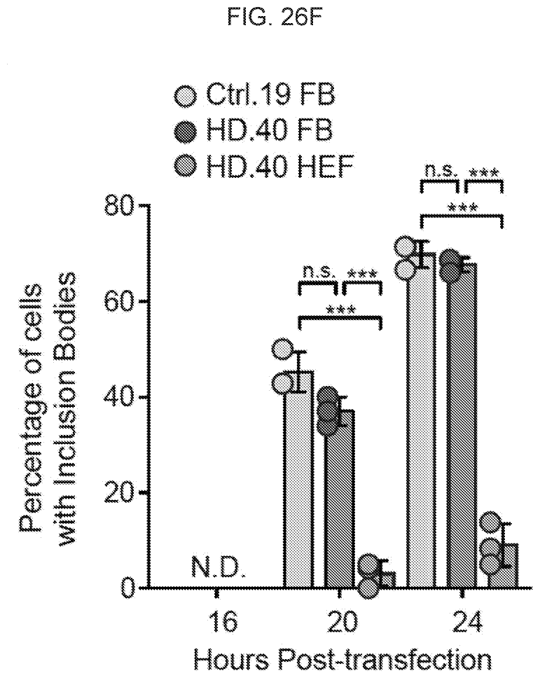

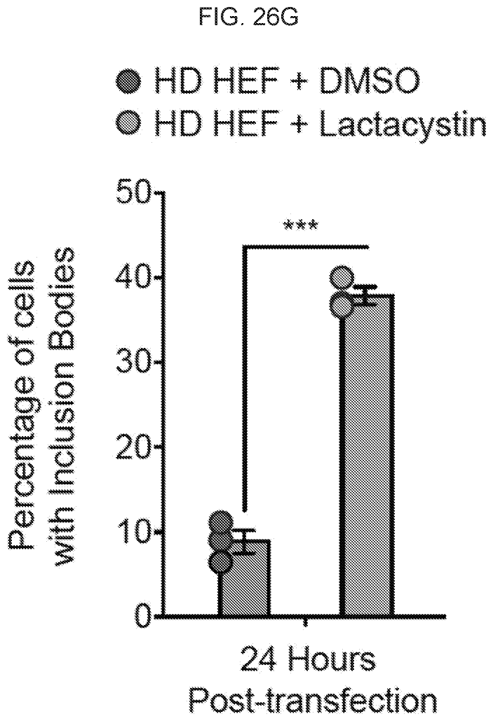

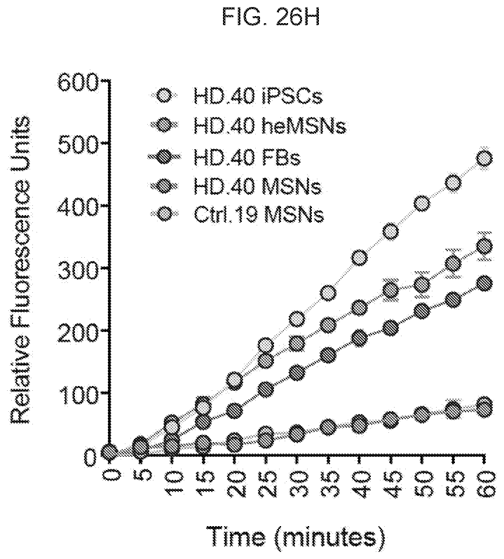

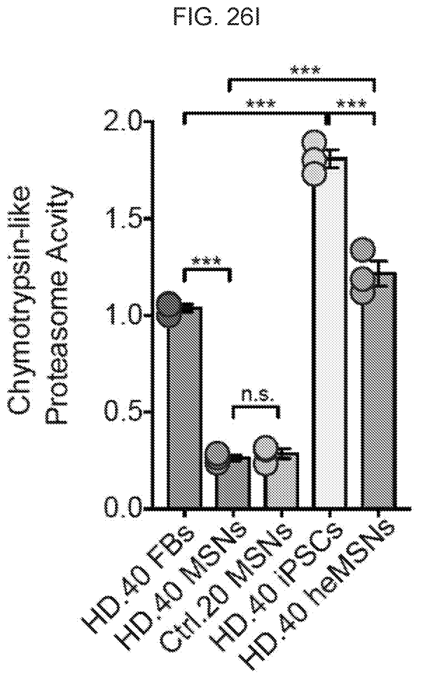

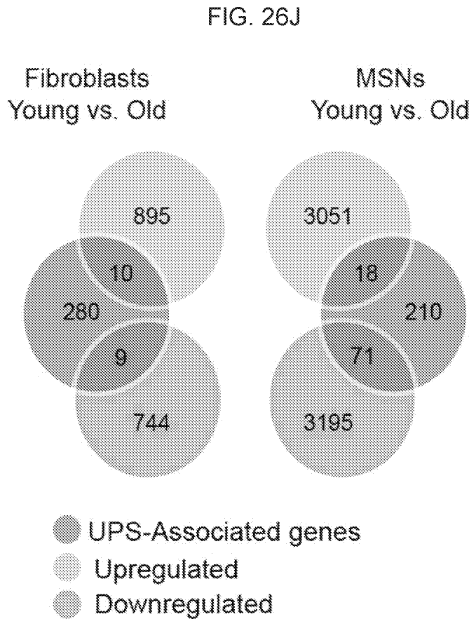

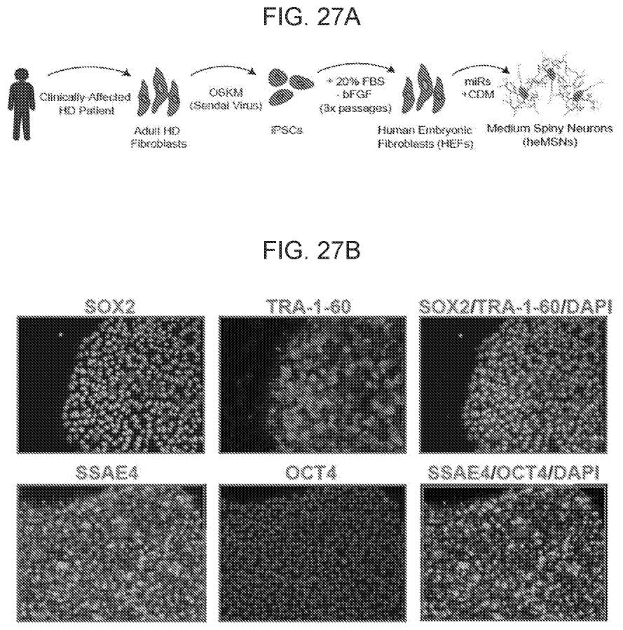

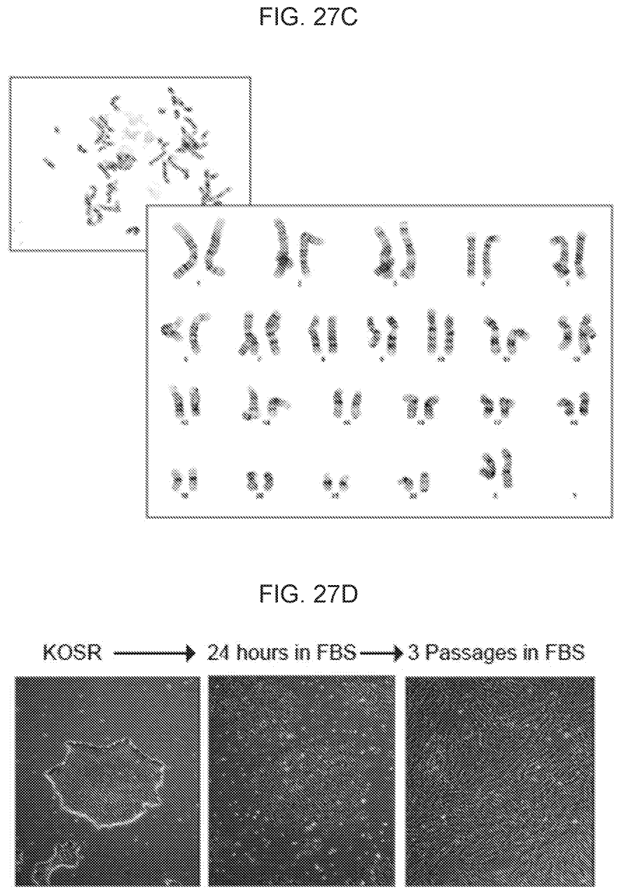

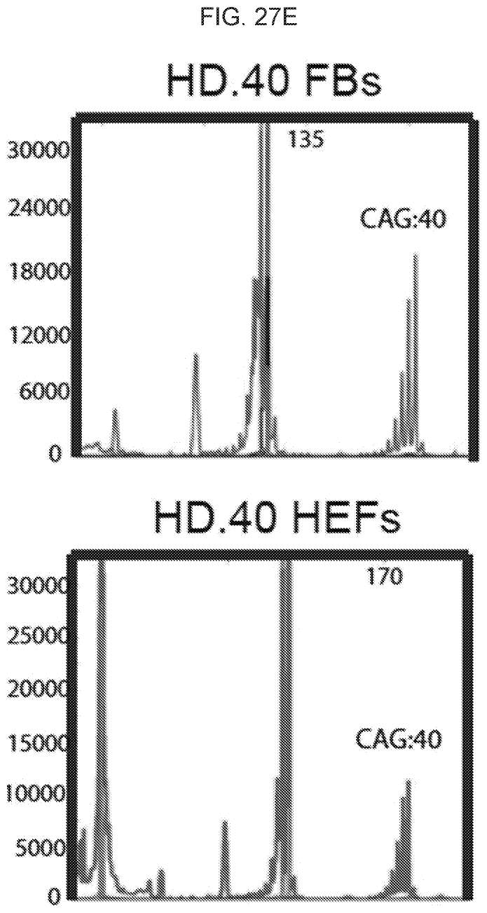

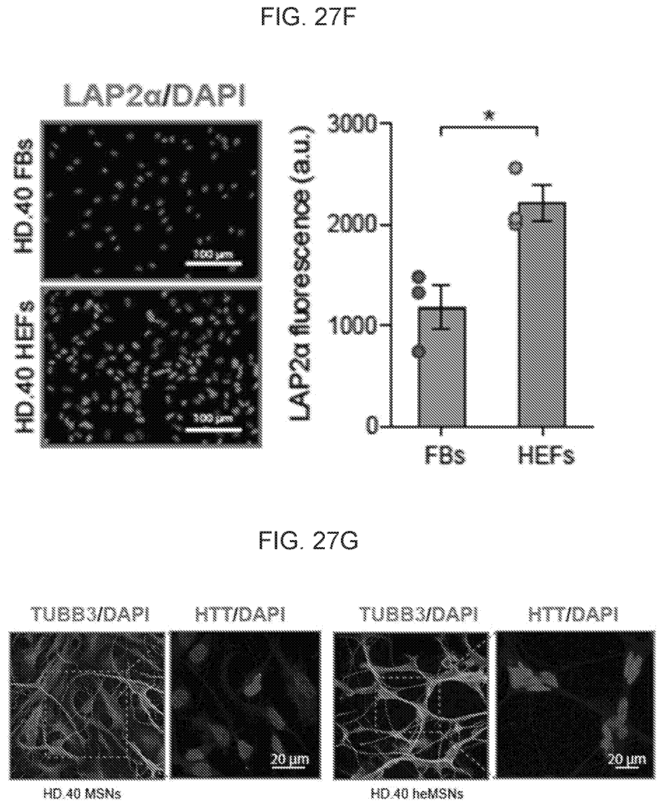

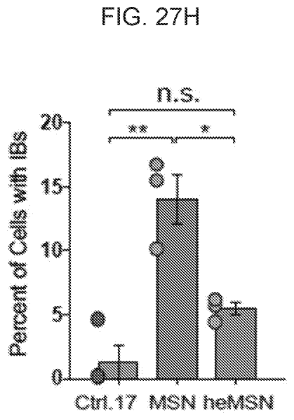

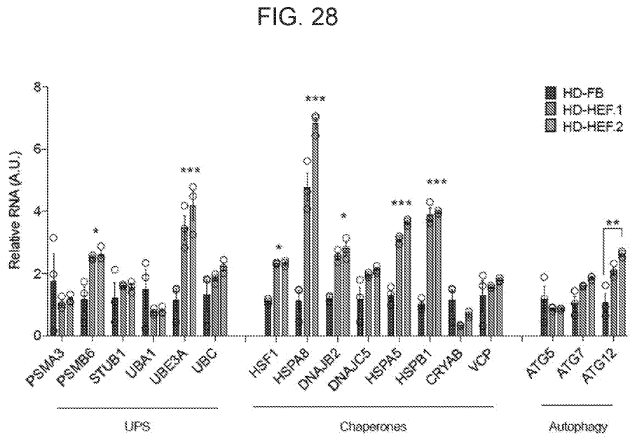

Because the findings contrasted with previous studies that report the lack of mHTT aggregates in iPSC-derived neurons from HD patients, it was decided to directly test if altering the cellular state of adult HD fibroblasts to an embryonic-like stage, a process that effectively erases aging markers, would affect the aggregation propensity of mHTT in HD-MSNs. HD-iPSCs were derived from adult HD fibroblasts and these stem cells were differentiated back into fibroblasts using a method for generating human embryonic fibroblasts (HEFs) (see e.g., FIG. 26A and FIG. 27A). Briefly, HD.40 fibroblasts were transduced with Sendai viral vectors to express the four reprogramming factors (Oct4, Sox2, Kfl4 and c-Myc), which resulted in integration-free iPSCs that expressed markers of pluripotency and retained a normal karyotype and CAG size (see e.g., FIG. 27B, FIG. 27C, FIG. 27D and FIG. 27E). HD.40-HEFs expressed fibroblast markers vimentin and fibronectin (see e.g., FIG. 26B). It was confirmed that HD.40-HEFs exhibited cellular markers typically associated with the reintroduction of an embryonic state, including high expression of the nuclear lamina-associated protein 2a (LAP2a) (see e.g., FIG. 27F). Upon direct conversion of HD.40-HEFs to MSNs (human embryonic MSNs, heMSNs), little to no aggregated mHTT was detectable in HD-heMSNs at PID 21 (see e.g., FIG. FIGS. 24B and 24C). These results were further verified using a different certified iPSC line from a symptomatic 37-year-old HD patient with 50 CAG repeats in HTT (see e.g., FIG. 27G and FIG. 27H). It was then decided to investigate differences in mHTT aggregation propensity to elucidate the contribution of aging to protein aggregation in HD. First, EGFP fused to 23 or 74 polyglutamine repeats (GFP 23Q or GFP-74Q) was ectopically expressed in either HD fibroblasts or HD-HEFs to track protein aggregation by live imaging (see e.g., FIG. 26E). While the expression of GFP-23Q stayed diffused, rapid rates of GFP-74Q aggregation (arrowheads in FIG. 26A, increased fluorescent density indicates the formation of inclusion bodies) were observed in aged fibroblasts with over 70% of HD fibroblasts displaying GFP-74Q aggregates after 24 hours, whereas in HEF cells, aggregates were only visible in fewer than 10% of HEFs at each time point analyzed (see e.g., FIG. 26F). Given that aggregates can be induced by treatment with proteasome inhibitors in iPSC-derived MSNs, it was postulated that higher proteasome activity was likely preventing GFP-74Q from forming aggregates in HEFs. qPCR analysis was performed for 17 genes associated with the Ubiquitin-Proteasome System (UPS), the main protein quality control machinery in the cell, in HD.40 fibroblasts and two iPSC clones of HD.40 differentiated into HEFs. It was found that 8 genes were consistently upregulated in HEFs while no genes tested were downregulated (see e.g., FIG. 28). Upregulated UPS genes included the heat-shock transcription factor HSF1, a protein that regulates the expression of genes involved in protein homeostasis. Studies with HD mouse models have previously shown that reducing the expression of HSF1 leads to increased HTT aggregation while overexpression of HSF1 inhibits polyQ aggregation. More recently, HSF1 protein was also shown to also be reduced in the striatum of HD patients. To directly test if proteasome activity was preventing the formation of inclusions in HEFs, GFP-74Q-expressing HEF cells were treated with the proteasome inhibitor lactacystin, and assessed the presence of inclusions 24 hours later. Lactacystin-treated HEFs had significantly increased numbers of cells bearing inclusions in contrast to DMSO treated HEFs (see e.g., FIG. 26C). These results collectively highlight the importance of maintenance of age during neuronal reprogramming in detecting mHTT aggregation phenotype.

Although iPSCs have been previously shown to possess higher proteasome activity than their originating fibroblasts, differentiation of iPSCs into neurons also was shown to reduce proteasome activity. Therefore, to determine if changes in proteasome activity could account for the detection of mHTT aggregation in MSNs but not in heMSNs, the functional activity of the proteasome was assessed with a fluorogenic peptide LLVY-AMC assay in converted neurons. Surprisingly, it was discovered that proteostasis was collapsed in HD-MSNs in comparison to heMSNs, which retained proteasome activity more comparable to iPSCs levels (see e.g., FIG. 26H and FIG. 26I). Importantly, significant changes in the aggregation propensity of GFP-74Q in fibroblasts from the 68-year old control in live imaging or proteasome functional activity were not detected, which indicates that aggregation propensity is not dependent on HTT mutation (data not shown). To explore if the collapse in proteostasis was an age-dependent process, the expression of 300 UPS-associated genes was analyzed generated by the transcriptional profiling of MSNs derived from microRNA-based conversion of fibroblasts of young (aged three days, five months and one year old) or old (aged 90, 92, and 92 years old) donors. By comparing gene expression in young versus old fibroblasts and MSNs, it was determined that even though fibroblasts do not display drastic changes in the expression of UPS-related genes with age, MSNs from older individuals show a dramatic increase in the number of downregulated UPS-related genes (see e.g., FIG. 26J) (list of 300 UPS-related genes and UPS-related genes that are differentially expressed in MSNs can be found on TABLE 4). In fact, gene ontology analysis of downregulated genes in old MSNs shows significant enrichment for the positive regulation of proteolysis (P-value of 2.01×10⁻²). These data suggest that the proteostasis collapse in MSNs, but not in originating fibroblasts or iPSC-derived neurons, is dependent on the cellular age of neurons.

TABLE 4

| | |
|---|---|
| UPS-related | AKTIP, ATG10, ATG101, ATG12, ATG13, ATG14, |
| genes | ATG16L1, ATG16L2, ATG2A, ATG2B, ATG3, ATG4A, |
| | ATG4B, ATG4C, ATG4D, ATG5, ATG7, ATG9A, |
| | ATG9B, BBS10, BBS12, BECN1, BIRC6, CCT2, CCT3, |
| | CCT4, CCT5, CCT6A, CCT6B, CCT7, CCT8, CDC34, |
| | CLPB, CRYAA, CRYAB, CYLD, DNAJA1, DNAJA2, |
| | DNAJA3, DNAJA4, DNAJB1, DNAJB11, DNAJB12, |
| | DNAJB13, DNAJB14, DNAJB2, DNAJB3, DNAJB4, |
| | DNAJB5, DNAJB6, DNAJB7, DNAJB8, DNAJB9, |
| | DNAJC1, DNAJC10, DNAJC11, DNAJC12, DNAJC13, |
| | DNAJC14, DNAJC15, DNAJC16, DNAJC17, DNAJC18, |
| | DNAJC19, DNAJC2, DNAJC21, DNAJC22, DNAJC24, |
| | DNAJC25, DNAJC27, DNAJC28, DNAJC3, DNAJC30, |
| | DNAJC4, DNAJC5, DNAJC5B, DNAJC5G, DNAJC6, |
| | DNAJC7, DNAJC8, DNAJC9, GABARAP, |
| | GABARAPL1, GABARAPL2, GAK, HSCB, HSP90AA1, |
| | HSP90AA3, HSP90AA3P, HSP90AB1, HSP90B1, |
| | HSPA12A, HSPA12B, HSPA13, HSPA14, HSPA1A, |
| | HSPA1B, HSPA1L, HSPA2, HSPA4, HSPA4L, HSPA5, |
| | HSPA6, HSPA7, HSPA8, HSPA9, HSPB1, HSPB11, |
| | HSPB2, HSPB3, HSPB6, HSPB7, HSPB8, HSPB9, |
| | HSPD1, HSPE1, HSPH1, HYOU1, MAP1LC3A, |
| | MAP1LC3B, MAP1LC3B2, MAP1LC3C, MKKS, |
| | MOCS3, NAE1, ODF1, PAN2, PIPSL, PSMA1, PSMA2, |
| | PSMA3, PSMA3P, PSMA4, PSMA5, PSMA6, PSMA7, |
| | PSMA7P, PSMA8, PSMB1, PSMB10, PSMB2, PSMB3, |
| | PSMB3P1, PSMB4, PSMB5, PSMB6, PSMB7, PSMB8, |
| | PSMB9, PSMC1, PSMC1P1, PSMC2, PSMC3, PSMC31P, |
| | PSMC3P1, PSMC4, PSMC5, PSMC6, PSMC6P1, |
| | PSMC6P2, PSMD1, PSMD10, PSMD10P1, PSMD10P2, |
| | PSMD10P3, PSMD11, PSMD12, PSMD12P, PSMD13, |
| | PSMD14, PSMD2, PSMD3, PSMD4, PSMD4P1, PSMD5, |
| | PSMD6, PSMD7, PSMD8, PSMD9, PSME1, PSME2, |
| | PSME2P1, PSME2P2, PSME2P3, PSME2P4, PSME2P5, |
| | PSME2P6, PSME3, PSME4, PSMF1, RB1CC1, SACS, |
| | SAE1, SEC63, SNX30, SNX4, STUB1, TCP1, TRAP1, |
| | UBA1, UBA2, UBA3, UBA5, UBA6, UBA7, UBE2A, |
| | UBE2B, UBE2C, UBE2D1, UBE2D2, UBE2D3, |
| | UBE2D4, UBE2E1, UBE2E2, UBE2E3, UBE2E4P, |
| | UBE2F, UBE2G1, UBE2G2, UBE2H, UBE2I, UBE2J1, |
| | UBE2J2, UBE2K, UBE2L1, UBE2L2, UBE2L3, |
| | UBE2L4, UBE2L5, UBE2L6, UBE2M, UBE2N, |
| | UBE2NL, UBE2Q1, UBE2Q2, UBE2R2, UBE2S, |
| | UBE2T, UBE2U, UBE2V1, UBE2V2, UBE2W, UBE2Z, |
| | ULK1, ULK2, USP1, USP10, USP11, USP12, USP13, |
| | USP14, USP15, USP16, USP17L2, USP18, USP19, |
| | USP2, USP20, USP21, USP22, USP24, USP25, USP26, |
| | USP27X, USP28, USP29, USP3, US930, USP31, USP32, |
| | USP33, USP34, USP35, USP36, USP37, USP38, USP39, |
| | USP4, USP40, USP41, USP42, USP43, USP44, USP45, |
| | USP46, USP47, USP48, USP49, USP5, USP50, USP51, |
| | U3P53, USP54, USP6, USP7, USP8, USP9X, USP9Y, |
| | USPL1, WIPI1, WIPI2 |
| UPS-related | ULK2, USP53, USP41, USP18, USP9X, USP6, UBE2T, |
| genes | UBE2L3, UBE2C, SAE1, MOCS3, CRYAB, HSPB6, |
| upregulated | HSP90AA1, HSPA12A, DNAJB8, DNAJB3, MKKS |
| in old MSNs | |
| (FC > 0.5 | |
| adjPvalue < | |
| 0.05) | |
| UPS-related | PSMA5, PSMA7, PSMB1, PSMB3, PSMB5, PSMB6, |
| genes | PSMB10, PSMC31P, PSMC4, PSMC5, PSMD2, PSMD8, |
| down- | PSMD9, PSMD11, PSME1, PSME4, CCT3, CCT7, |
| regulated | CLPB, DNAJA2, DNAJA3, DNAJB1, DNAJB2, |
| in old MSNs | DNAJB5, DNAJB12, DNAJC5, DNAJC8, DNAJC11, |
| (FC < −0.5 | DNAJC13, HSCB, GAK, HSPA2, HSPA4, HSP90AB1, |
| adjPvalue < | TRAP1, HSPB1, UBA1, UBA6, UBA7, BIRC6, UBE2B, |
| 0.05) | UBE2D2, UBE2D3, UBE2E3, UBE21, UBE2J2, UBE2M, |
| | UBE2NL, UBE2Q1, UBE2R2, CYLD, USP5, USP11, |
| | USP19, USP24, USP28, USP30, USP34, USP35, USP37, |
| | USP40, PAN2, ULK1, ATG4D, BECN1, GABARAP, |
| | MAP1LC3A, ATG9A, ATG13, WIPI2, ATG101 |

mHTT-Mediated DNA Damage and Spontaneous Degeneration

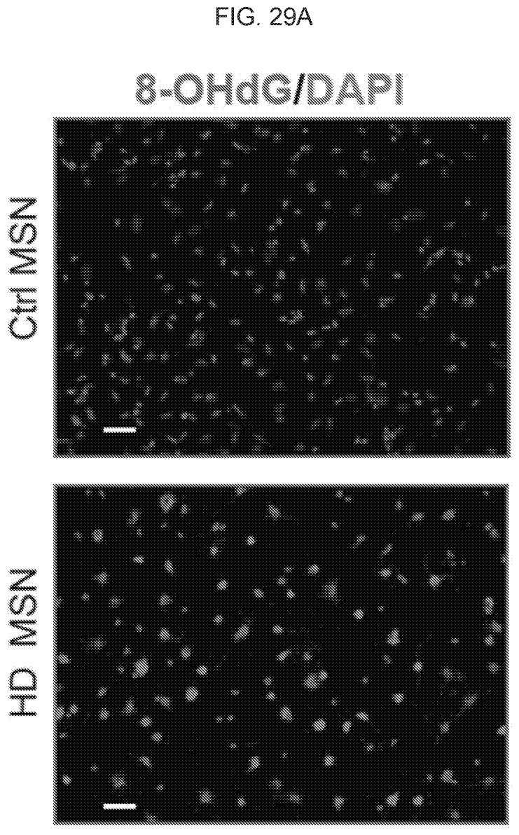

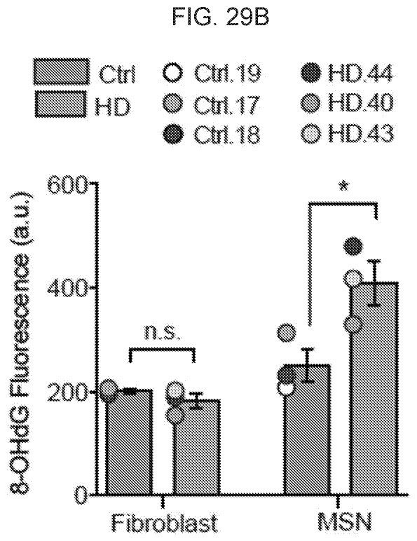

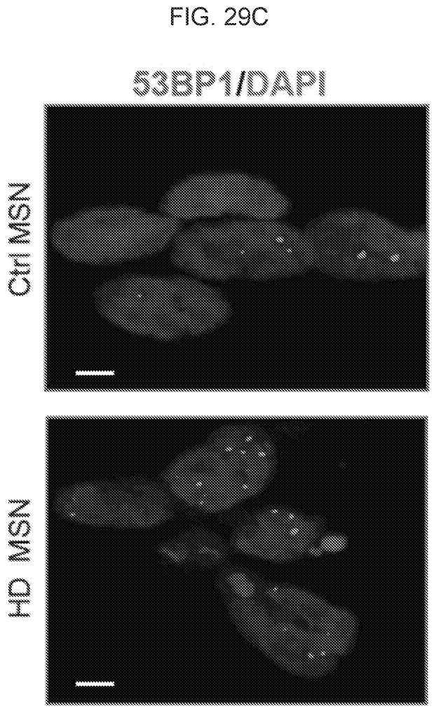

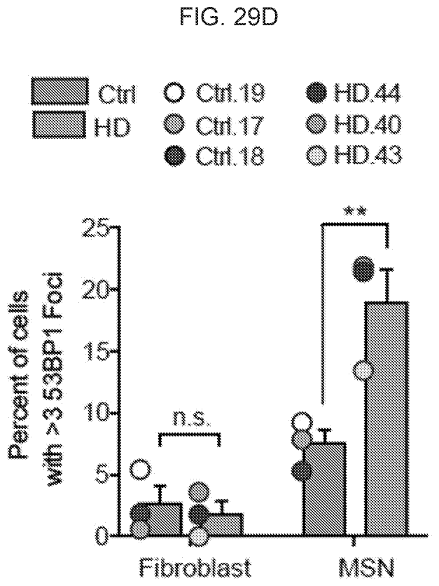

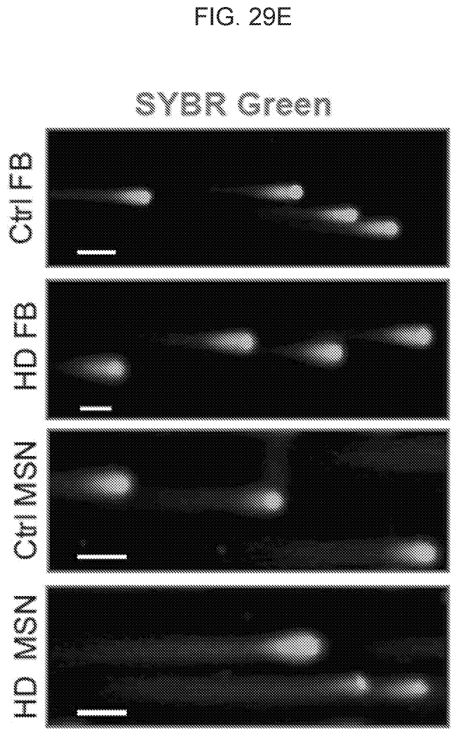

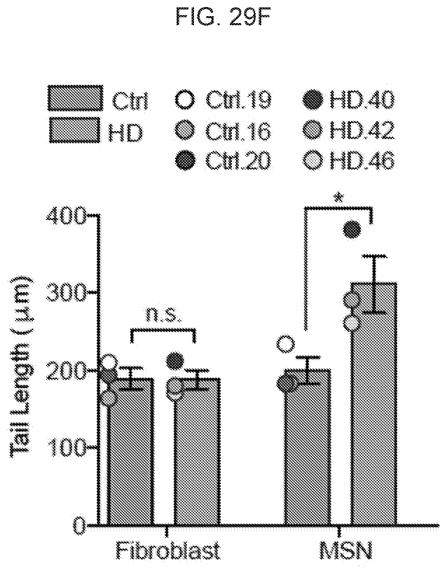

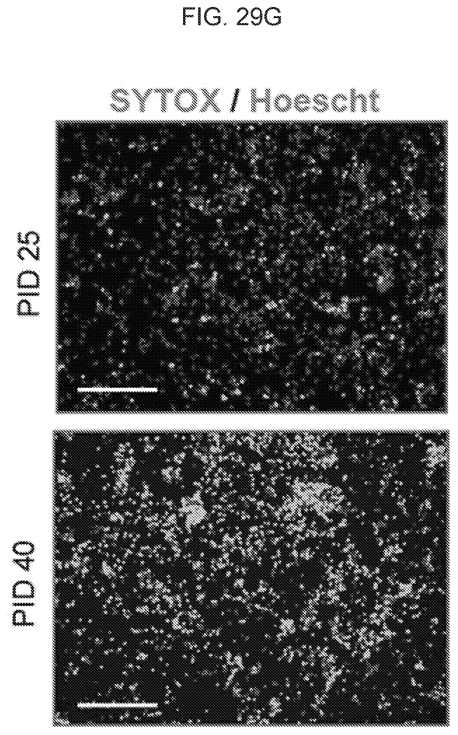

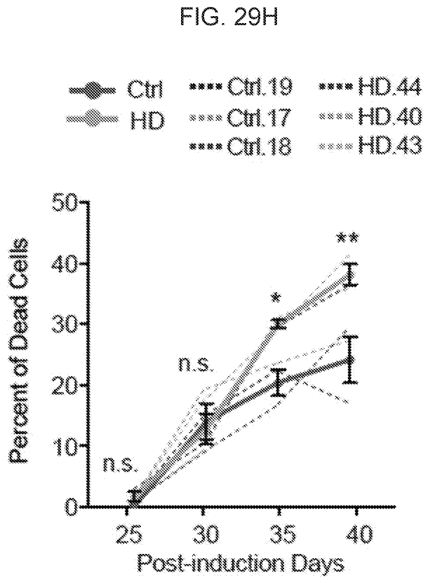

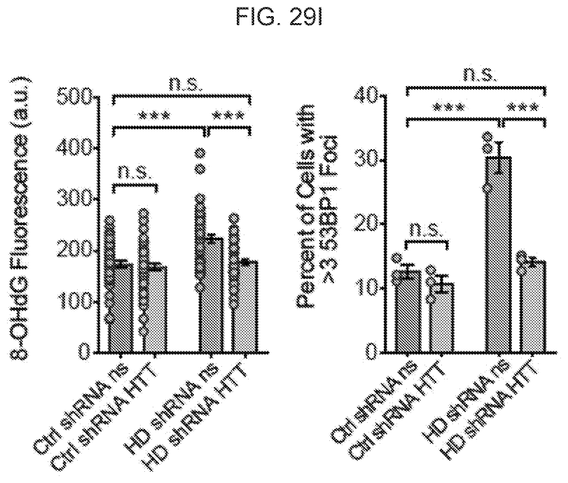

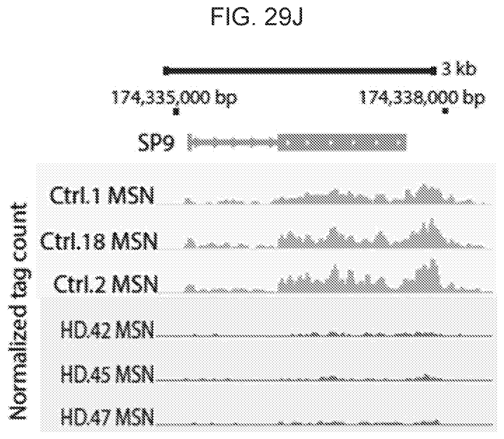

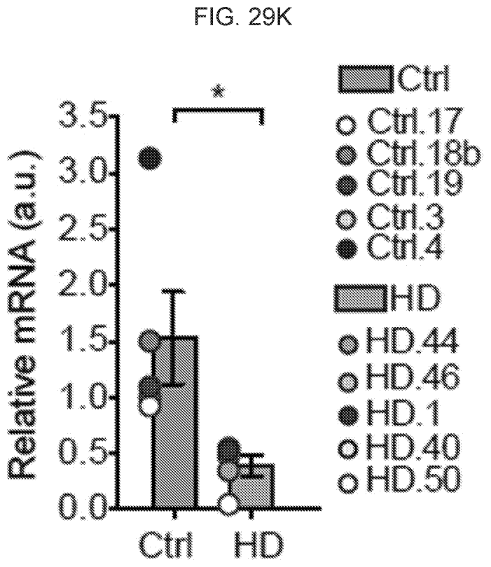

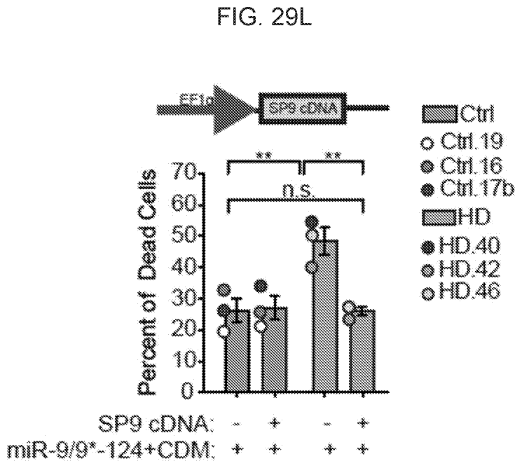

Because aging contributes to the onset of HD, it was decided to test if direct conversion would also offer advantages in modeling spontaneous degeneration of HD patient's MSNs, a phenotype that has not been previously described in iPSC-derived neurons from adult-onset HD patients with low repeat numbers. DNA damage was first measured in HD-MSNs converted from three independent HD patients in comparison to starting fibroblasts and Ctrl-MSNs. At PID 30, HD-MSNs exhibited increased oxidative DNA damage determined by levels of 8-hydroxy-2′-deoxyguanosine (8-OHdG) (see e.g., FIG. 29A and FIG. 29B), as well as increased double-stranded breaks assessed by the presence of nuclear 53BP1 foci (see e.g., FIG. 29C and FIG. 29D). Furthermore, analysis by single-cell gel electrophoresis (also known as comet assay) that visualizes the migration of broken DNA strands from individual agarose-embedded cells showed a marked increase in comet tail lengths in comparison to Ctrl-MSNs at PID 30. No significant difference in comet tail lengths was detected between HD and control fibroblasts (see e.g., FIG. 29E and FIG. 29F). Next, levels of spontaneous cel death was quantified in three controlled pairs of HD- and Ctrl-MSNs using SYTOX green, a nucleic acid stain impermeable to live cells, at multiple time-points during reprogramming (see e.g., FIG. 29G and FIG. 29H). Cell death levels were comparable between HD- and Ctrl-MSNs until PID 30, but differed drastically for all HD-MSNs in relation to their controls at PID 35 and 40 (see e.g., FIG. 29H), also evidenced by drastic reduction of DARPP-32-positive HD-MSNs (see e.g., FIG. 30). The detected DNA damage was dependent on HTT as AAV-mediated reduction of HTT significantly reduced 8-OHdG levels and 53BP1 foci number in HD-MSNs (see e.g., FIG. 29I). In order to test if the cell death phenotype in HD-MSNs was amenable to pharmacological intervention and given that it was preceded by extensive DNA damage, the effect of ataxia-telangiectasia mutated (ATM) kinase inhibitor (KU60019) was examined on cellular degeneration in HD-MSNs. ATM is a central regulator of the DNA damage response and is selectively activated to induce apoptosis upon DNA damage or oxidative stress. Inhibiting ATM levels with KU60019 has been found to reduce mHTT-induced cell death. Consistent with these findings, the treatment of HD-MSNs with 0.5 μM of KU60019 significantly reduced levels of spontaneous and stress-induced cell death (see e.g., FIG. 31). Next, it was tested whether restoring the expression of SP9, a zinc finger transcription factor recently shown to be critical for the survival of MSNs⁴¹ and identified to be significantly downregulated in HD-MSNs in RNA-seq analysis presented herein, could prevent spontaneous degeneration of HD-MSNs. First, the detected reduction of SP9 expression in HD-MSNs from our RNA-seq analysis was validated by qPCR in five independent HD-MSN samples, and a nearly 4-fold decrease in SP9 expression was observed (see e.g., FIG. 29J and FIG. 29K). The cDNA of SP9 was then cloned downstream of the human EF1α promoter in a lentiviral vector to allow consistent expression of SP9 in reprogramming cells. At PID 14 of miR-9/9*-124+CDM reprogramming, three HD-MSN (HD.40, HD.42, and HD.46) and three Ctrl-MSN (Ctrl. 16, Ctrl. 17b and Ctrl. 19) samples were transduced with lentivirus carrying the SP9 cDNA construct and cultured until PID 35, when levels of cell death were assayed with SYTOX green. Restoring SP9 expression in HD-MSNs reduced cell death to levels indistinguishable from controls (see e.g., FIG. 29L). Although loss of SP9 has been previously shown to lead to apoptosis of MSNs in mice, further studies are needed to probe the neuroprotective mechanism of this transcription factor and its potential role in HD pathogenesis. At the very least, the results herein indicate that directly converted HD-MSNs may serve as a useful platform for the screening of pharmacological and genetic factors that may have a therapeutic potential for treating HD.

Mitochondrial Dysfunction, Oxidative Stress and Metabolic Deficits in HD-MSNs

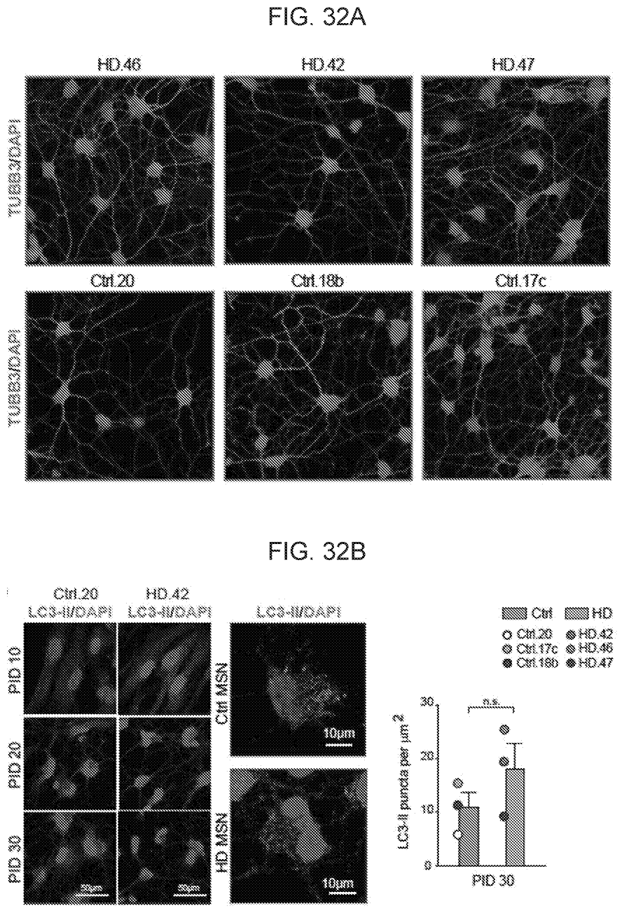

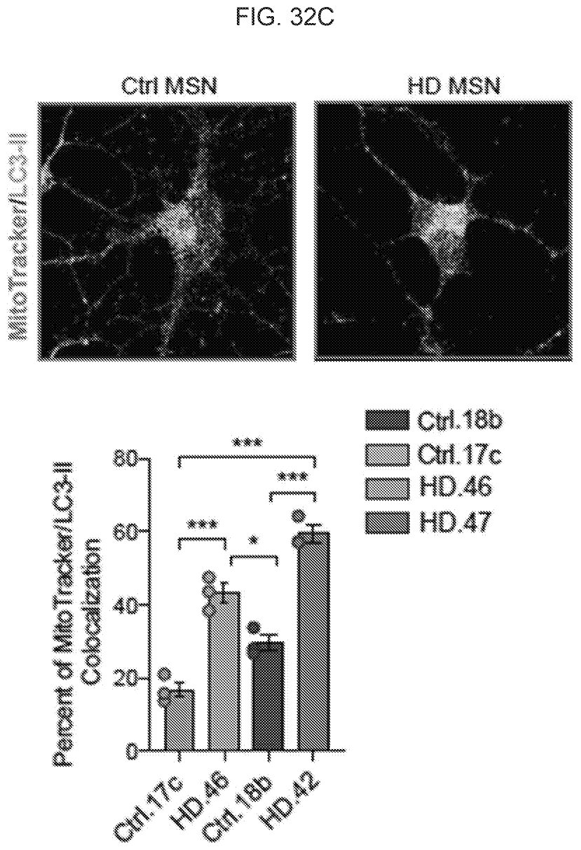

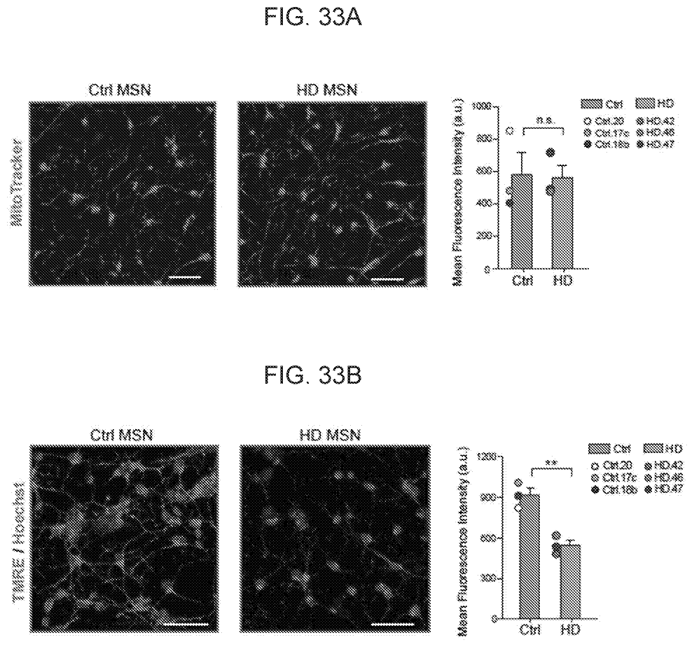

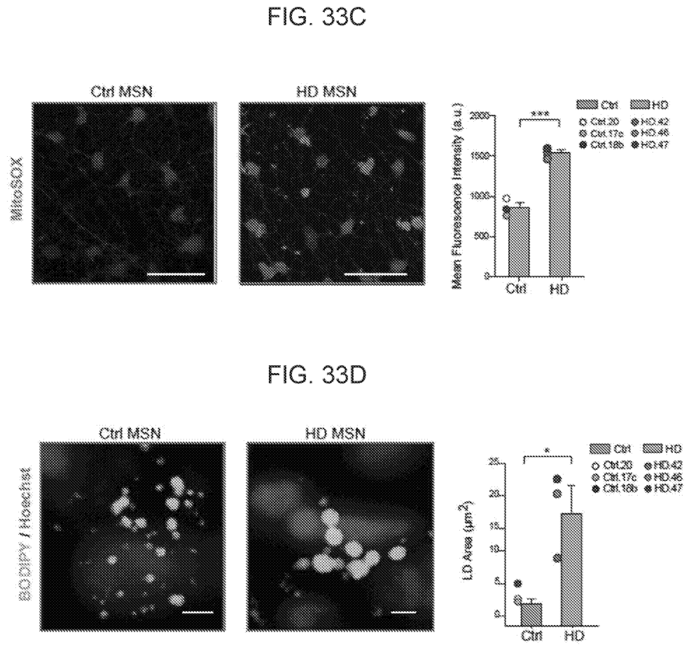

TEM analysis of HD-MSNs and Ctrl-MSNs (HD.40 and Ctrl. 19) identified high levels of mitophagy, the selective degradation of dysfunctional mitochondria, and many swollen mitochondria typical of apoptotic cells (see e.g., FIG. 24E). The accumulation of cytoplasmic lipid droplets was observed, a process that leads to neurodegeneration and is caused by oxidative stress and mitochondrial dysfunction (see e.g., FIG. 24E). Additionally, there was an enrichment of lipofuscin granules in HD-MSNs, which are aging pigments that accumulate due to incomplete lysosomal degradation of damaged mitochondria⁶¹ and are a known cellular defect induced by mHTT as identified in animal and human postmortem studies (see e.g., FIG. 24C). To gain a better insight into the mitochondrial and metabolic dysfunctions present in reprogrammed HD-MSNs, six lines (HD.42, HD.46, and HD.47; Ctrl. 19, Ctrl. 20, Ctrl. 17c and Ctrl. 18b—see e.g., FIG. 32) were reprogrammed and the previous observations were systematically quantified (see e.g., FIG. 33 and FIG. 24). First, the total pool of mitochondria was determined using the mitochondrial indicator MitoTracker Red between HD- and Ctrl-MSNs and no significant differences were found (see e.g., FIG. 33A). Next changes to the mitochondrial membrane potential were assessed with TMRE, an indicator of active and polarized mitochondria, and significantly lower levels of TMRE signal were detected, indicating decreased membrane potential of mitochondria in HD-MSNs (see e.g., FIG. 33B). Increased production of reactive oxygen species (ROS) by mitochondria is thought to be a major cause of oxidative stress in HD and a critical component in the progression of the disease⁸³. Live imaging of HD-MSNs with the superoxide indicator, MitoSOX Red, revealed significantly higher levels of ROS in comparison to controls (see e.g., FIG. 33C). To quantify our initial observation by TEM of lipid droplet accumulation, the presence of lipids was measured using the lipid dye BODIPY 498/503 and it was found that HD-MSNs had significantly larger lipid droplets in comparison to controls (see e.g., FIG. 33D). Furthermore, the accumulation of lipofuscin granules was confirmed across multiple HD lines by performing additional TEM in six lines (HD.42, HD.46, and HD.47; Ctrl. 20, Ctrl. 16 and Ctrl. 15) (see e.g., FIG. 24C). Since these results indicate impaired mitochondrial health, levels of mitophagy in HD-MSNs were explored. Reprogrammed MSNs were labeled with MitoTracker and immunostained for the autophagosome marker, LC3-II, for colocalization analysis. A greater percentage of mitochondria and autophagosome colocalization was observed in HD-MSN lines (see e.g., FIG. 32C) suggesting an increase in mitophagy. Of note, HD patients and mouse models have been shown to display an increase in the number of autophagosomes. Two out of three HD-MSNs derived from independent patients showed increased expression in LC3-II immunoreactive cytoplasmic puncta over controls, however overall the changes were not statistically significant (see e.g., FIG. 32B). Collectively, these results recapitulate mitochondrial and metabolic dysfunctions in HD-MSNs observed in HD models and patients.

Differential Vulnerability of Neuronal Subtypes to mHTT Toxicity

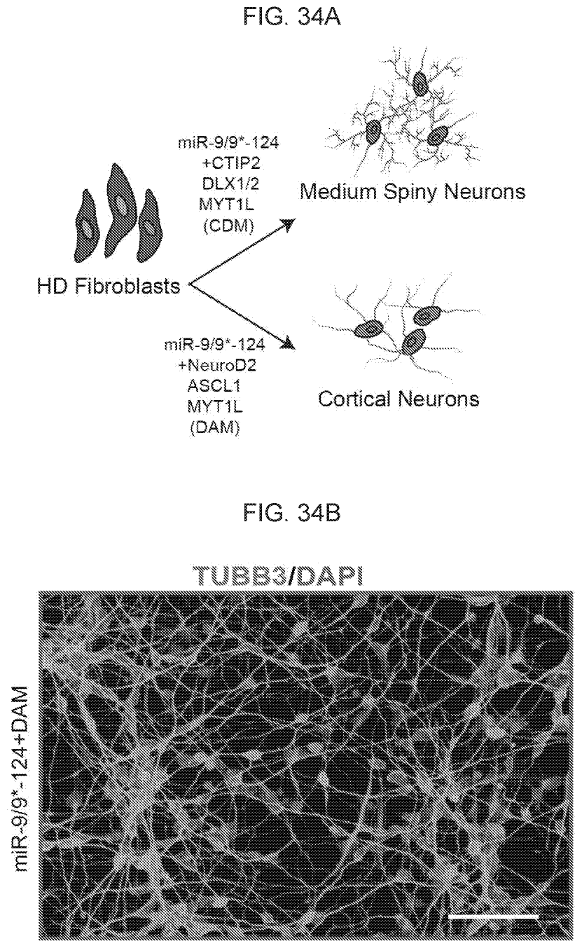

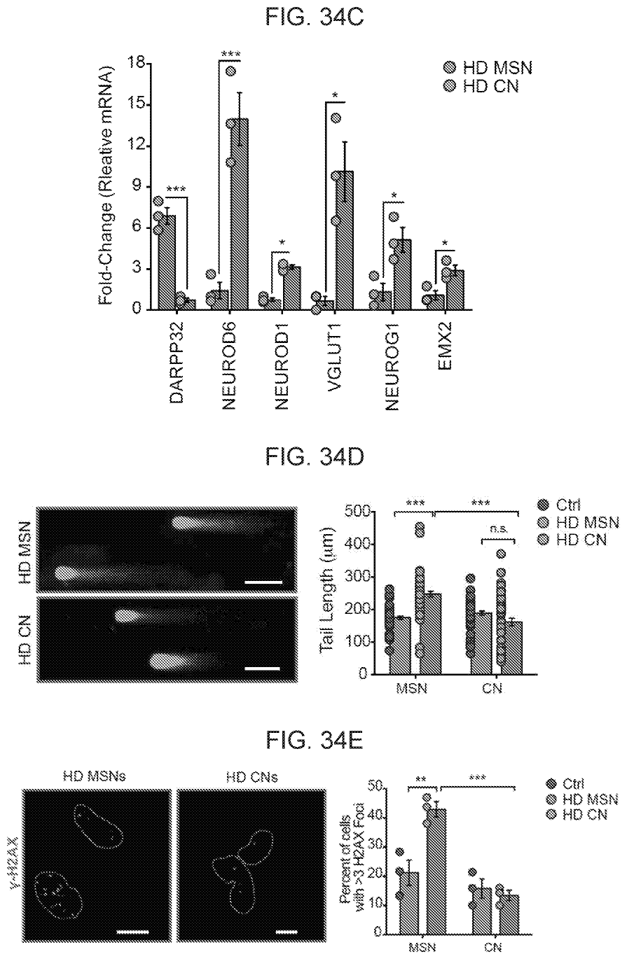

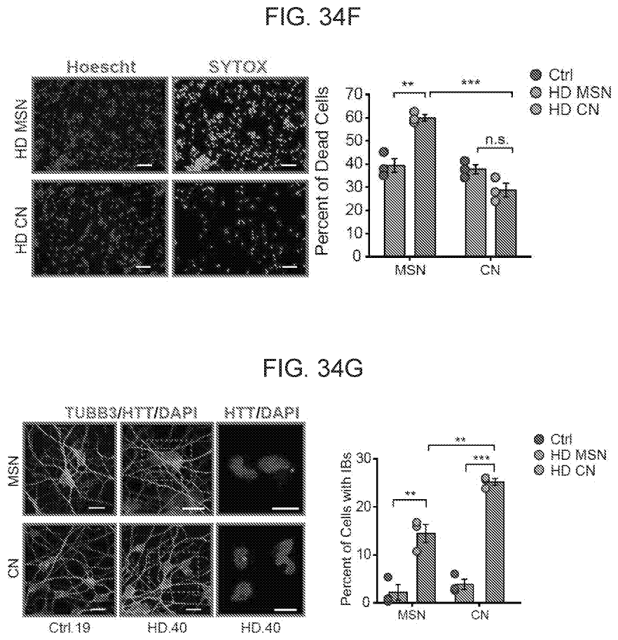

Although HTT is ubiquitously expressed throughout the brain, mHTT leads to selective mass degeneration of MSNs and to a lesser extent, cortical neurons as the disease progresses. Human postmortem studies have shown that at a stage when neuronal loss was low in the cortex but high in the striatum, mHTT aggregates were more common in the cortex than in the striatum³⁷. Additionally, the formation mHTT inclusion bodies was also reported to correlate positively with neuronal survival and hence may be a protective cellular response. It was hypothesized that by generating cortical neurons (CNs) from HD fibroblasts (HD-CNs), the selective vulnerability of HD-MSNs can be modeled with directly reprogrammed human neurons and the relationship could be examined between aggregate formation and toxicity. Control and HD-patient fibroblasts were transduced either with miR-9/9*-124+CDM or with miR-9/9*-124 in conjunction with NeuroD2, ASCL1 and MYT1L (DAM) (miR-9/9*-124+DAM), a combination that has been shown to convert human fibroblasts into neurons that express markers associated with cortical neurons (see e.g., FIG. 34A). Surprisingly, levels of DNA damage were lower in HD-CNs (see e.g., FIG. 34D and FIG. 34E). Moreover, HD-CNs exhibited a lower magnitude of cell death in comparison to HD-MSNs (see e.g., FIG. 34F). Interestingly, the fraction of neurons displaying mHTT aggregates was higher in HD-CNs than in HD-MSNs (see e.g., FIG. 34G). This suggests that cellular properties intrinsic to MSNs render them differentially vulnerable in HD pathology.

Manifestation of HD Cellular Phenotypes is Dependent on Patient Age

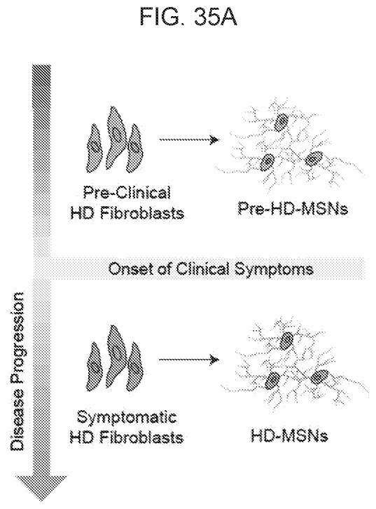

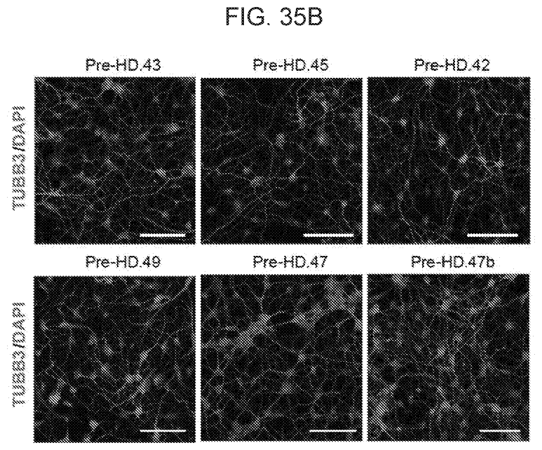

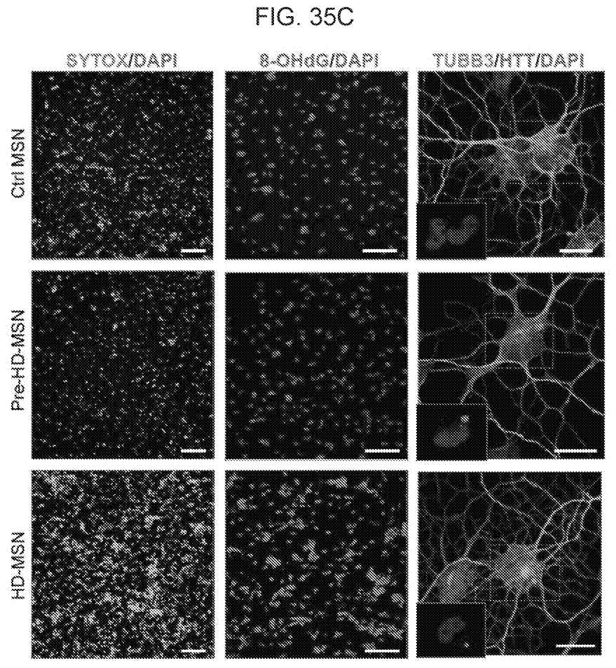

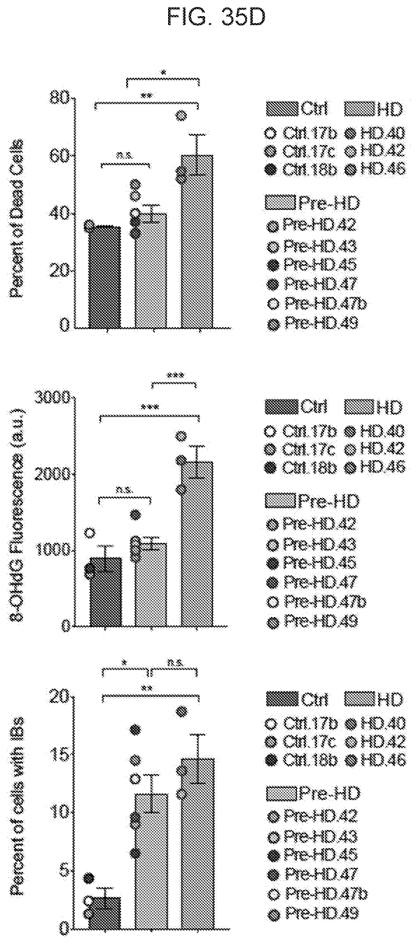

Unlike iPSC-derived neurons, directly converted neurons do not undergo rejuvenation during cell fate conversion. The maintenance of aging signatures upon neuronal conversion has long been postulated to be an important advantage of using directly converted patient neurons to model late-onset diseases. However, no functional studies have provided empirical evidence that age information stored within donor's somatic cells actually contributes to the differential manifestation of HD-related cellular phenotypes. Even though the findings of deriving HD-heMSNs were insightful, the induction of pluripotency is likely altering many cellular properties and not just erasing aging signatures. Therefore, the ability to attribute the importance of aging to phenotypic manifestation of HD in our cellular model is limited. To further evaluate the importance of cellular age to our phenotypic analyses, the properties of MSNs reprogrammed from HD patients but sampled before the disease onset were investigated. Six fibroblasts lines were acquired from pre-symptomatic HD patients (Pre-HD), sampled 13 to 17 years prior to the onset of clinical symptoms, with CAG tract sizes of 42-49 repeats (see e.g., TABLE 2). All six Pre-HD fibroblasts were reprogrammed using miR-9/9*-124+CDM to generate MSNs (Pre-HD-MSNs), alongside fibroblasts from three controls and three symptomatic HD patients (see e.g., FIG. 35A). Representative images of TUBB3-stained Pre-HD-MSNs show the successful adoption of neuronal fate from these fibroblasts lines (see e.g., FIG. 35B). At PID 35, Pre-HD-MSNs were less vulnerable to mHTT-induced toxicity with lower levels of cel death (SYTOX Green, see e.g., FIG. 35C—left column) and oxidative DNA damage (80H-dG, see e.g., FIG. 35C—middle column, quantification in FIG. 35D). Notably, Pre-HD-MSNs still contained mHTT aggregations at a similar level to symptomatic HD-MSNs (EM48 mHTT, see e.g., FIG. 35C—right column, quantification in FIG. 35D). These results are of great importance given that they directly show the age-dependent onset of HD can be modeled with directly converted HD-MSNs and provide a human cellular model for examining the contribution of age and genetic factors to disease onset.

### DISCUSSION

The ability to model neurological disorders and neuronal function in human neurons in vitro has proved to be a valuable approach for dissecting disease pathogenesis. However, since many neurological disorders primarily affect distinct neuronal subpopulations, studies using generic protocols to induce unrestricted neuronal cell fates are likely only capturing a global snapshot of factors that contribute to disease onset and progression. This is especially true for the study of HD, in which MSNs are differentially susceptible to cell death. The microRNA-based neuronal conversion for generating MSNs offers an experimental means to generate a highly enriched population of human MSNs from HD patients.

In HD, the accumulation of protein aggregates and neurodegeneration is observed in an age-dependent manner. Moreover, forced expression of mHTT leads to more severe pathological changes in the striatum of old rats than in young rats, including increased aggregate load and striatal cell loss⁵⁸. Several other lines of evidence in HD patients and animal models suggest that deficits caused by HD pathogenesis are age-related, such as mitochondrial dysfunction, oxidative stress, and DNA damage⁵⁹. To test the contribution of cellular age to the manifestation of disease-relevant phenotypes in MSNs derived from patient fibroblasts, two distinct cellular reprogramming approaches were applied that diverge in the maintenance of age signatures from donor cells. The induction of pluripotency has been well established to erase aging marks and reset the phenotypic age of donor cells to an embryonic state, while direct conversion has been shown to maintain age-related transcriptional, cellular and epigenetic signatures. In this study, it was established that the retention of age status through direct neuronal conversion is a critical component in effectively modeling HD, demonstrated by the detection of mHTT aggregates and determination that the propensity of mHTT to aggregate is directly related to the age- and cell fate-related functionality of proteostasis.

It was also found that mHTT induced DNA damage contributed to the cell death of HD-MSNs, as treating the cells with an inhibitor of the DNA damage response protein ATM rescued the cell death phenotype and protected the cells against oxidative stress, similar to iPSC-derived neurons undergoing degeneration upon BDNF withdrawal. Moreover, evidence was provided that generating MSNs with high specificity is critical for the manifestation of disease phenotypes, as altering the terminal neuronal cell fate of HD fibroblasts to HD-CNs drastically reduced levels of DNA damage and cell death, despite the presence of mHTT aggregates. Although cortical cells are not spared in HD, it has been observed that cortical neurons degenerate at a much slower rate with disease progression relative to MSNs and that mHTT aggregates are more common in the cortex than in the striatum. Accordingly, postmortem studies in HD patients have also shown significantly lower levels of DNA damage in the cortex than in the striatum. The cellular properties that render MSNs differentially vulnerable to mHTT-induced toxicity are poorly understood. The reprogramming approach described herein offers a platform to examine neuroprotective attributes conferred by acquisition of cortical fate, an important aim of further studies. Finally, the mechanistic roles of DNA damage response pathways in the modification of HD pathogenesis remain largely unknown. Importantly, recent human genetic studies reveal crucial DNA damage repair pathway gene loci (e.g. FAN1 and MLH1) are significantly associated with altered onset of motor symptoms in HD. The robust MSN-specific DNA repair pathway deficits in this HD-MSN model in conjunction with phenotype-free MSNs derived from pre-symptomatic HD patients may offer a new patient-derived neuronal paradigm to study human genetic modifier genes for HD.

Plasmids and Lentiviral Preparation

The construction of all plasmids used in this study has been previously described^{19,63} and are publicly available at Addgene: pTight-9-124-BclxL (#60857), rtTA-N144 (#66810), pmCTIP2-N106 (#66808), phMYT1L-N174 (#66809), phDLX1-N174 (#66859), and phDLX2-N174 (#66860). With the exception of hSP9-N174 which was cloned in house and not in prior publications. Polyglutamine fusion proteins, pEGFP-23Q and pEGFP-74Q were generated and acquired by from Addgene (#40261 and #40262), and transfected into human fibroblasts. Lentiviral production was carried out separately for each plasmid but transduced together as a single cocktail as previously described⁶³. Briefly, supernatant was collected 60-70 hours after transfection of Lenti-X 293LE cells (Clontech) with each plasmid, in addition to psPAX2 and pMD2.G (Addgene), using polyethyleneimine (Polysciences). Collected lentiviruses were filtered through 0.45 μm PES membranes and concentrated at 70,000×g for 2 hours at 4° C. Viral pellets were re-suspended in 1× Dulbecco's phosphate-buffered saline (DPBS, Gibco) and stored at −80° C. until transduction.

Cell Lines and Culture

Adult dermal fibroblasts of symptomatic HD patients (Coriell NINDS and NIGMS Repositories: ND33947, ND30013, GM02173, GM09197, GM04687, GM04230, GM04194, GM04196, GM04198, GM02147. GM04687) and healthy controls (Coriell NINDS, NIA, and NIGMS Repositories: ND34769, AG04148, GM02171, GM05879, AG16409, AG11357, AG11483, GM05879, AG16409, AG05265, AG04062, AG04060) were acquired from the Coriell Institute for Medical Research. One additional healthy control adult dermal fibroblast line was acquired from the Washington University School of Medicine iPSC Core Facility (#F09-238). The International Cell Line Authentication Committee (ICLAC) lists none of these primary cells as commonly misidentified cell lines. In regards to de-identified skin fibroblasts samples and induced pluripotent stem cells (iPSCs) acquired from the Coriell Institute for Medical Research, the master list to re-identify subjects was not accessible. This activity is not considered to meet federal definitions under the jurisdiction of an Institutional Review Board, and thus exempt from the definition of human subject. All fibroblasts were cultured in fibroblast media (FM): Dulbecco's Modified Eagle Medium (DMEM) with high glucose containing 15% fetal bovine serum (FBS; Gibco), 0.01% β-mercaptoethanol (BME), 1% non-essential amino acids (NEAA), 1% sodium pyruvate, 1% GlutaMAX, 1% 1M HEPES buffer solution and 1% penicillin/streptomycin solution (all from Invitrogen). Cell cultures are routinely checked and confirmed to be free of mycoplasma contamination. The step-by-step MSN conversion protocol has been previously presented⁶³. Briefly, the lentiviral cocktail of rtTA, pTight-9-124-BclxL, CTIP2, MYT1L, DLX1, and DLX2 was added to fibroblasts for 16 hours, then cells were washed and fed with FM with 1 μg/mL doxycycline (DOX). Cells were fed at post-induction day (PID) 3 with FM+puromycin (3 μg/mL)+blasticidin (3 μg/mL)+DOX and re-plated PID 5 onto poly-omithine/fibronectin/laminin-coated glass coverslips in FM+DOX. Media was switched PID 6 to Reprogramming Neuronal Medium (RNM): Neuronal Medium (NM; ScienCell Research Laboratories) with 200 μM dibutyl cyclic AMP, 1 mM valproic acid, 10 ng/mL BDNF, 10 ng/mL NT-3, and 1 μM retinoic acid, supplemented with DOX. Half volume media changes with RNM were performed every 4 days with addition of DOX every 2 days thereafter until PID 30-35. Addition of puromycin and blasticidin was terminated after PID 14.

DNA Extraction and CAG Sizing

Fibroblasts were expanded in culture, collected by cell scraper, pelleted, and lysed for DNA extraction and ethanol precipitated following typical lab procedures with Proteinase K (Roche). DNA samples were CAG sized by Laragen, Inc (Culver City, Calif.).

Immunocytochemistry

Cells were fixed using 4% paraformaldehyde (PFA) for 20 minutes and permeabilized using 0.2% Triton-X solution for 10 minutes following three phosphate-buffered saline (PBS) washes. Cells were blocked for 1 hour at room temperature using 1% Normal Goat Serum (NGS) and 5% bovine serum albumin (BSA) in 1×PBS solution. Primary antibodies were added in the presence of blocking buffer overnight at 4° C. Secondary antibodies were added following three PBS washes at 1:1000 in blocking buffer at room temperature for 1 hour. The following primary antibodies were used for the immunofluorescence studies: mouse anti-MAP2 (Sigma-Aldrich #M9942 Clone HM2, 1:750), rabbit anti-β-III tubulin (BioLegend, #MMS-435P, 1:2,000), chicken anti-NeuN (Aves, #NUN, 1:500), rabbit anti-GABA (Sigma #A2052, 1:2,000), mouse anti-GABA (Sigma #A0310 Clone GB-69, 1:500), rabbit anti-DARPP32 (Santa Cruz Biotechnology #so-11365, 1:400), rabbit anti-S100A4 (FSP1) (Abcam #124805, 1:200), mouse anti-HTT (mEM48, Millipore #MAB5374, 1:50) (MW8, Developmental Studies Hybridoma Bank, 1:100), rabbit anti-ubiquitin (Abcam #ab7780, 1:50), mouse anti-vimentin (Sigma-Aldrich #V6630, 1:500), rabbit anti-fibronectin (Sigma-Aldrich #F3648, 1:500), mouse anti-phospho-histone H2A.X (Millipore #05-636-I, 1:200), rabbit anti-lap2 alpha (Abcam #ab5162, 1:500), rabbit anti-53BP1 (Abcam #ab21083, 1:200), mouse anti-80H-dG (Santa Cruz Biotechnology #so-139586, 1:1,000), rabbit anti-LC3B (Sigma-Aldrich #L7543, 1:1,000). The secondary antibodies were goat anti-rabbit or mouse IgG conjugated with Alexa-488, -594, or -647 (Invitrogen). Images were captured using a Leica SP5X white light laser confocal system with Leica Application Suite (LAS) Advanced Fluorescence 2.7.3.9723. All staining quantification was performed by counting number of positive-stained cells over DAPI signal. Antibodies were validated by staining fibroblasts as negative controls, and exhibited low background.

Immunoblot Analysis

At post-infection day 28, cells were lysed in SDS-Lysis buffer (1 M Tris-HCl pH 6.8, 2% SDS, 30% Glycerol) supplemented with protease inhibitors (Roche, #04693132001). The concentrations of whole cell lysates were measured using the Pierce BCA protein assay kit (Thermo Scientific, #23227). Equal amounts of whole cell lysates were resolved by SDS-PAGE and transferred to a nitrocellulose membrane (GE Healthcare Life Sciences, #10600006) using a transfer apparatus according to the manufacturer's protocols (Bio-rad). After incubation with 5% BSA in TBS containing 0.1% Tween-20 (TBST) for 30 min, the membrane was incubated with primary antibodies at 4° C. overnight; MW8 (Developmental Studies Hybridoma Bank, 1:500) and MW1 (Developmental Studies Hybridoma Bank, 1:500). Following incubation, membranes were incubated with a horseradish peroxidase-conjugated anti-mouse or anti-rabbit antibody for 1 hr. Blots were developed with the ECL system (Thermo Scientific, #34080) according to the manufacturer's protocols.

Mitochondrial Assays

The cell permeant mitochondrial indicator, MitoTracker Red CMXRos (ThermoFisher Scientific #M7512) was added directly to live cells at final concentration of 50 nm in serum-free media. After 20 minutes of incubation in 37° C., cells were imaged with an epifluorescent microscope and then fixed and processed for immunostaining as described above. Analysis of colocalization of MitoTracker Red and LC3-II (Anti-LC3B antibody, Sigma-Aldrich #L7543) was performed using Metamorph bioimaging software after image acquisition using a Leica SP5X white light laser confocal system with Leica Application Suite (LAS). Mitochondrial membrane potential was assayed with TMRE-Mitochondrial Membrane Potential Assay Kit (abcam #ab113852) following the manufacturer's protocol. Briefly, TMRE was added to live cells at a final concentration of 20 nm in serum-free media. After 15 minutes of incubation in 37° C., coverslips were removed from media and Vaseline was applied to edges of coverslips to create a rim for live mounting and microscopy (Fischer et al., CSH Protocols, 2008) and imaged using a Leica SP5X white light laser confocal system with Leica Application Suite (LAS). Lipid droplets were stained with BODIPY 493/503 (4,4-Difluoro-1,3,5,7,8-Pentamethyl-4-Bora-3a,4a-Diaza-s-Indacene) (ThermoFisher Scientific #D3922) at a final concentration of 0.1 μm in serum-free media. After 30 minutes of incubation in 37° C., cells were imaged with an epifluorescent microscope and quantified with Leica Application Suite (LAS) quantification tools.

Electrophysiology

Whole-cell patch-clamp recordings were performed 28-35 days post-induction (PID) with miR-9/9*-124-CDM. At PID 14, cells undergoing reprogramming were transduced with pSYNAPSIN tRFP or GFP, and the next day trypsinized and plated together on top of rat primary neurons and glia isolated from perinatal pups with the exception of recordings shown in FIG. 20H which were performed in monoculture in the absence of rat primary cells. Fluorescent reporter expression was visible within days, and remained segregated for each population. Data was acquired using pCLAMP 10 software with multiclamp 700B amplifier and Digidata 1550 digitizer (Molecular Devices). Electrode pipettes were pulled from borosilicate glass (World Precision Instruments) and typically ranged between 4-6 MC resistance. Solutions used to study intrinsic neuronal properties were the same as previously reported (Victor et al., Neuron 2014). Post-synaptic potentials were detected spontaneously. Data was collected in Clampex and initially analyzed in Clampfit (Molecular Devices).

RNA Extraction and Gene Expression Profiling

Total RNA was extracted and isolated with TRIzol reagent (Thermo Fisher Scientific) according to manufacturer's instructions. cDNA was generated from isolated RNA with Superscript III Reverse Transcriptase (Thermo Fisher Scientific) primed with random hexamers. qPCR was performed with the following primer sets listed in TABLE 5. For RNA-seq, reads were aligned to the human genome (assembly hg38) with STAR version 2.4.2a [23104886]. Gene counts were derived from the number of uniquely aligned unambiguous reads by Subread:featureCount [23558742], version 1.4.6, with GENCODE gene annotation (V23) [22955987]. All gene-level transcript counts were then imported into the RBioconductor package EdgeR [19910308] and TMM normalized to adjust for differences in library size. Genes not expressed in any sample were excluded from further analysis. The fit of the trended and tagwise dispersion estimates were then plotted to confirm proper fit of the observed mean to variance relationship where the tagwise dispersions are equivalent to the biological coefficients of variation of each gene. Differentially expressed genes were then filtered for those having fold-changes (FC)>1.5 together with false-discovery rate (FDR) adjusted p-values less than or equal to 0.05. Gene expression heat maps were generated using Z-scores for expression values of each gene among different samples (GENE-E Matrix Visualization and Analysis Platform, Broad Institute). MSN-specific genes were selected from previous studies that have profiled transcriptome profiles of various neuronal subtypes^{64,65}. RNA-seq data is publicly available at GEO (Accession number GSE84013).

TABLE 5

| Target | |
|---|---|
| (FD-Forward, | |
| RV-Reverse) | Primer Sequence (5′-3′) |
| EMX2 FD | CGGCACTGAGICTACGCTAAC |
| EMX2 FD | CAAGTCCGGGTTGGAGTAGAC |
| NEUROG1 FD | GCTCTCTGACCCCAGTAGC |
| NEUROG1 FD | GCGTTGTGTGGAGCAAGTC |
| NEUROD1 FD | GCCGCAGGGTTATGAGACTA |
| NEUROD1 RV | TCTGTCCAGGTTGGAGGAC |
| NEUROD6 FD | ACACTACCGTTTGATGAGTCTGT |
| NEUROD6 FD | CTTCTGGTCCTCGCATTCTCT |
| DARPP-32 FD | TCTGAAGTCG.GAGAGGCAACs |
| DARRP-32 RV | IGGAGGTGAGADTGAGGAA |
| HSPA8 FD | ACCTACTCTTGTGTGGGTGTT |
| HSPA8 RV | GACATAGCTTGGAGTGGTTCG |
| HSPA5 FD | CATCACGCCGTCCTATGTCG |
| HSRA5 RV | CGTCAALGACCGTGTTCTCG |
| DNAJB2 FD | ATGGCATCCTACTACGAGATCC |
| DNAJB2 RV | GAGAGCCTTGCGCCGATAC |
| DNAJC5 FD | GGGAGTCATTGTACCACGTCC |
| DNAJC5 RV | CGTGCGCGTTGTTGATCTC |
| HSRPB1 FD | ACGGTCAAGACCAAGGATGG |
| HSPB1 RV | AGCGTGTATTTCCGCGTGA |
| HSF1 FD | CCATGAAGCATGAGAATGAGGC |
| HSF1 RV | CTTGTTGACGACTTTCTGTTGC |
| ATG5 FD | AAAGATGTGCTTCGAGATGTGT |
| ATG5 RV | CACTTTGTCAGTTACCAACGTCA |
| ATG7 FD | CAGTTTGCCCCTTTTAGTAGTGC |
| ATG7 RV | CCAGCCGATACTCGTTCAGC |
| ATG12 FD | CTGCTGGCGACACCAAGAAA |
| ATG12 RV | CGTGTTCGCTCTACTGCCC |
| CRYAB FD | CCTGAGTCCCTTCTACCTTCG |
| CRYAB RV | CAGATCTCCCAACACCTTAACTT |
| VCR FD | CAAACAGAAGAACCGTCCCAA |
| VCP RV | TCACCTCGGAACAACTGCAAT |
| STUB1 FD | AGCAGGGCAATCGTCTGTTC |
| STUB1 RV | CAAGGCCCGGTTGGTGTAATA |
| RPS27A FD | CTGGAAGATGGACGTACTTTGTC |
| RP527A RV | CGADGAAGGCGACTAATTTTGC |
| UBA1 FD | TCGCCGCTGTCCAAGAAALC |
| UBA1 RV | AGPAAAGGCCCTCGTCTATGTC |
| URE3A FD | CTCAGCTTACCTTGAGAACTCG |
| UBE3A RV | TTCTAGCGCCTTTCTTGTTCAT |
| PSMA3 FD | GCTCAATCGGCACTGGGTAT |
| PSMA3 RV | ACCTGCTACTGCCATTCCAAC |
| PSMB6 FD | GGCTACCTTACTAGCTGCTCG |
| PSMB6 RV | GATTGGCGATGTAGGACCCAG |

Dead-Cell Staining

SYTOX Green nucleic acid staining (Thermo Fisher Scientific) was performed following manufacturer's suggestions, and adapted as follows: A final concentration of 0.1 μM SYTOX green was added directly to the media of live cells. In addition, Hoechst 33342 solution (Thermo Fisher Scientific) was added as a counterstain to label all nuclei at a final concentration of 1 μg/ml in culture media. Samples were incubated for at least 10 minutes in 37° C. Images were captured using a Leica DMI 400B inverted microscope with Leica Application Suite (LAS) Advanced Fluorescence. Three images were taken from random areas of each coverslip for at least three biological replicates per experiment. Quantification performed by counting number of SYTOX-positive cells over total Hoechst signal.

Comet Assay

DNA damage was assessed by using the CometAssay® reagent kit for single cell gel electrophoresis assay (Trevigen, Md. USA), following the recommended protocol for neutral conditions, and adapting the gel electrophoresis methods for use in the Sub-Cell GT electrophoresis system (Bio-Rad, CA USA). Briefly, cells were collected from coverslips by treatment with 0.25% trypsin, pelleted and resuspended at 100,000 cells/ml in 1×DPBS (Ca²⁺ and Mg²⁺ free; Thermo Fisher Scientific) and verified to be greater than 95% viable by tryptan blue exclusion using an automated cell counter before continuing analysis. Approximately 5,000 cells were embedded in low melting agarose, plated on slides and lysed overnight. The next day, electrophoresis was run at 30 Volts for 30 minutes in 1×TBE (National Diagnostics). Samples were fixed in 70% ethanol for 5 minutes, and slides were immersed in 1×TE buffer pH 8.0 (Ambion) with 1:10 of 10,000×SYBR green nucleic acid stain (Thermo Fisher Scientific). Fluorescent images were captured using a Leica DMI 400B inverted microscope for scoring.

Generation of iPSCs and Derivation of HEFs

iPSC lines used in this study were either directly acquired from the Coriell Institute for Medical Research NINDS Biorepository (#ND42235) or derived from adult dermal fibroblast acquired from the Coriell NINDS Biorepository (#ND33947) with the assistance of the Washington University School of Medicine Genome Engineering and iPSC Center (GEiC). For the generation of ND33947 iPSCs, fibroblasts were transduced with integration-free Sendai reprogramming vectors for Oct3/4, Sox2, Klf4, and c-Myc and characterized by the expression of the pluripotency markers Oct4, SSEA4, SOX2 and TRA-1-60 (PSC 4-Marker Immunocytochemistry Kit, Molecular Probes). Cytogenic analysis was performed on twenty G-banded metaphase cells from iPSC line at passage 5 and all twenty cells demonstrated an apparently normal karyotype (Cell Line Genetics, Madison Wis.). In addition, embryoid body formation assay confirmed the potential for acquisition of all three germ layers. iPSCs were expanded on ES grade Matrigel (Corning) coated plates cultured in mTeSR medium (STEMCELL Technologies) or DMEM/F-12 with 20% KnockOut Serum Replacement, 1% GlutaMAX, 0.1 mM NEAA, 10 ng/mL fibroblast growth factor-basic (bFGF) and 55 μM BME. To differentiate iPSCs into human embryonic fibroblasts (HEFs), culture media was replaced with DMEM+20% FBS without bFGF for at least three passages. HEFs were transduced and reprogrammed to MSNs following the established previously reported protocol (Richner, 2015).

Drug Treatment

The ATM-Kinase inhibitor KU-60019 was obtained from Abcam (ab144817), solubilized in DMSO and directly added to the cell culture media for a final concentration of 0.5 μM at 30 days post miR-9/9*-124 induction, then cell death was assessed by SYTOX at PID 35. Controls were treated with the same volume of DMSO but no drug. At day 35, cells treated with DMSO or KU-60019 also were treated with 1 mM of H₂O₂ for three hours. SYTOX green/Hoechst stain was added as already described and imaged for scoring.

20s Proteasome Activity Assay

Adherent cells were dissociated with 0.25% trypsin, pelleted by centrifugation and washed in cold 1×PBS twice. Cell pellets were then resuspended in chilled cell lysis buffer (50 mM HEPES (pH 7.5), 5 mM EDTA, 150 mM NaCl, 1% Triton X-100, and 2 mM ATP) and incubated on ice for 30 minutes, and vortexed every 10 minutes. Cell lysate were then centrifuged at 15,000 RPM for 15 minutes at 4° C. Lysate was then transferred to a microcentrifuge tube, and 10 μL of each sample was used to determine protein concentration with a BCA protein assay kit (Thermo Scientific, Prod. #23227) following manufacturer's recommendations. Proteasome activity was assayed with 10 μg of each lysate with a 20 s Proteasome activity assay kit (Millipore, APT280). Fluorescent intensity was measured every 5 minutes for 1 hour with a microplate reader. Data was analyzed following previously reported methods⁴⁹.

Electron Microscopy

Cells cultured in gridded glass bottom μ-dishes (Ibidi, Madison, Wis.) were fixed with EM grade 4% PFA+0.05% glutaraldehyde (GA) (Electron Microscopy Sciences) in 1×PBS with 2 mM CaCl₂ at 37° C. for 5 minutes (min) then transferred to ice for 1 hour. Samples were then incubated for 5 min in 50 mM glycine in 1×PBS and permeabilized with 0.05% saponin with 1% BSA in PBS for 30 min. Cells were blocked with 1% BSA in PBS for 15 min and incubated with primary antibodies (mouse anti-HTT (MW8), 1:100 and rabbit anti-R-III tubulin BioLegend, 1:2,000) at room temperature for 2 hours with gentile agitation. After washing in PBS-BSA three times for 10 min each, cells were incubated for an additional 2 hours with Alexa Fluor 594 fluoronanogold secondary antibody (Nanoprobes, Yaphank, N.Y.) at a 1:250 dilution in PBS and 1% BSA at room temperature with gentle agitation while wrapped in foil. After washing in PBS three times for 10 min each, cells were fixed with 1% GA for 5 min and labeled with DAPI (1:10,000) for 5 min. Post fluorescent imaging, the samples were rinsed twice in ultrapure water for 1 min each and then rinsed in 0.02 M citrate buffer (pH 4.8) three times for 5 min each. The fluoronanogold label was silver enhanced using HQ Silver (Nanoprobes, Yaphank, N.Y.) for 9-11 min and immediately rinsed with ultrapure water twice for 5 min each. The culture dishes were then rinsed in PBS buffer three times for 10 minutes each, and subjected to a secondary fixation step for one hour in 1% osmium tetroxide/0.3% potassium ferrocyanide in PBS on ice. The samples were then washed in ultrapure water three times for 10 minutes each and then en bloc stained for 1 hour with 2% aqueous uranyl acetate. After staining was complete, samples were briefly washed in ultrapure water, dehydrated in a graded ethanol series (50%, 70%, 90%, 100%×2) for 10 minutes in each step, and infiltrated with microwave assistance (Pelco BioWave Pro, Redding, Calif.) into LX112 resin. Samples were cured in an oven at 60° C. for 48 hours. Once the resin was cured, the gridded glass coverslips were etched away with concentrated hydrofluoric acid and the exposed cells were excised with a jewelers saw and mounted onto blank resin blocks with epoxy, oriented in the coverslip growing plane. 70 nm thick sections were then taken and imaged on a TEM (JEOL JEM 1400 Plus, Tokyo, Japan) at 80 KeV.

Statistics

For all quantified data, multiple cells were counted from at least three biological replicates from multiple independent experiments or multiple lines. Statistical analyses were performed in GraphPad Prism using a two-tailed Students t-test or a one-way ANOVA followed by a post hoc Tukey's test with *P<0.05 considered significant Multiple comparisons were corrected with Bonferroni or Holm-Sidak method as described in the figure legends. Studies were performed blindly and automated whenever possible with the aid of ImageJ cell counting tools, and multiple investigators confirmed quantification results. Normality was tested with D'Agostino-Pearson test. In addition, Brown-Forsythe test did not identify significantly different standard deviations (P<0.05) for groups tested. Data in graphs are expressed as mean and error bars represent s.e.m. unless noted otherwise. Outliers were detected and excluded with Grubbs' test for alpha levels of 0.05. In total for this study, only 2 data points were excluded from FIG. 31A and FIG. 31B control DMSO group (9 total data points) that met the pre-established criteria.

Step-by-step protocols used herein can be found in Richner, M., Victor, M. B., Liu, Y., Abernathy, D. & Yoo, A. S. MicroRNA-based conversion of human fibroblasts into striatal medium spiny neurons. Nature protocols 10, 1543-1555, doi:10.1038/nprot.2015.102 (2015).

### REFERENCES

1 Gusella, J. F. et al. A polymorphic DNA marker genetically linked to Huntington's disease. Nature 306, 234-238 (1983).
2 A novel gene containing a trinucleotide repeat that is expanded and unstable on Huntington's disease chromosomes. The Huntington's Disease Collaborative Research Group. Cell 72, 971-983 (1993).
3 Beighton, P. & Hayden, M. R. Huntington's chorea. S Afr Med J 59, 250 (1981).
4 Kremer, B. et al. A worldwide study of the Huntington's disease mutation. The sensitivity and specificity of measuring CAG repeats. N Engl J Med 330,1401-1406, doi:10.1056/NEJM199405193302001 (1994).
Warby, S. C. et al. CAG expansion in the Huntington disease gene is associated with a specific and targetable predisposing haplogroup. Am J Hum Genet 84, 351-366, doi:10.1016j.ajhg.2009.02.003 (2009).
6 Brinkman, R. R., Mezei, M. M., Theilmann, J., Almqvist, E. & Hayden, M. R. The likelihood of being affected with Huntington disease by a particular age, for a specific CAG size. Am J Hum Genet 60, 1202-1210 (1997).
7 Ross, C. A. et al. Huntington disease: natural history, biomarkers and prospects for therapeutics. Nat Rev Neurol 10, 204-216, doi:10.1038/nmeurol.2014.24 (2014).
8 Arrasate, M., Mitra, S., Schweitzer, E. S., Segal, M. R. & Finkbeiner, S. Inclusion body formation reduces levels of mutant huntingtin and the risk of neuronal death. Nature 431, 805-810, doi:10.1038/nature02998 (2004).
9 Vonsattel, J. P. & DiFiglia, M. Huntington disease. J Neuropathol Exp Neurol 57, 369-384 (1998).
10 HD iPSC Consortium. Induced pluripotent stem cells from patients with Huntington's disease show CAG-repeat-expansion-associated phenotypes. Cell Stem Cell 11, 264-278, doi:10.1016j.stem.2012.04.027 (2012).
11 Zhang, N., An, M. C., Montoro, D. & Ellerby, L. M. Characterization of Human Huntington's Disease Cell Model from Induced Pluripotent Stem Cells. PLoS Curr2, RRN1193, doi:10.1371/currents.RRN1193 (2010).
12 Arber, C. et al. Activin A directs striatal projection neuron differentiation of human pluripotent stem cells. Development 142, 1375-1386, doi:10.1242/dev.117093 (2015).
13 An, M. C. et al. Genetic correction of Huntington's disease phenotypes in induced pluripotent stem cells. Cell Stem Cell 11, 253-263, doi:10.1016/j.stem.2012.04.026 (2012).
14 Jeon, I. et al. Neuronal properties, in vivo effects, and pathology of a Huntington's disease patient-derived induced pluripotent stem cells. Stem cells 30, 2054-2062, doi:10.1002/stem.1135 (2012).
15 Nekrasov, E. D. et al. Manifestation of Huntington's disease pathology in human induced pluripotent stem cell-derived neurons. Molecular neurodegeneration 11, 27, doi:10.1186/s13024-016-0092-5 (2016).
16 Camnasio, S. et al. The first reported generation of several induced pluripotent stem cell lines from homozygous and heterozygous Huntington's disease patients demonstrates mutation related enhanced lysosomal activity. Neurobiology of disease 46, 41-51, doi:10.1016J.nbd.2011.12.042 (2012).
17 Yoo, A. S. et al. MicroRNA-mediated conversion of human fibroblasts to neurons. Nature 476, 228-231, doi:10.1038/nature10323 (2011).
18 Yoo, A. S., Staahl, B. T., Chen, L. & Crabtree, G. R. MicroRNA-mediated switching of chromatin-remodelling complexes in neural development. Nature 460, 642-646, doi:10.1038/nature08139 (2009).
19 Victor, M. B. et al. Generation of human striatal neurons by microRNA-dependent direct conversion of fibroblasts. Neuron 84, 311-323, doi:10.1016/j.neuron.2014.10.016 (2014).
20 Miller, J. D. et al. Human iPSC-based modeling of late-onset disease via progerin-induced aging. Cell Stem Cell 13, 691-705, doi:10.1016j.stem.2013.11.006 (2013).
21 Horvath, S. DNA methylation age of human tissues and cell types. Genome Biol 14, R115, doi:10.1186/gb-2013-14-10-r115 (2013).
22 Huh, C. J. et al. Maintenance of age in human neurons generated by microRNA-based neuronal conversion of fibroblasts. Elife 5, doi:10.7554/eLife.18648 (2016).
23 Mertens, J. et al. Directly Reprogrammed Human Neurons Retain Aging-Associated Transcriptomic Signatures and Reveal Age-Related Nucleocytoplasmic Defects. Cell Stem Cell 17, 705-718, doi:10.1016/j.stem.2015.09.001 (2015).
24 Liu, Y. et al. Direct reprogramming of Huntington's disease patient fibroblasts into neuron-like cells leads to abnormal neurite outgrowth, increased cell death, and aggregate formation. PLoS One 9, e109621, doi:10.1371journal.pone.0109621 (2014).
25 Xie, Y. Z. & Zhang, R. X. Neurodegenerative diseases in a dish: the promise of iPSC technology in disease modeling and therapeutic discovery. Neurological sciences: official journal of the Italian Neurological Society and of the Italian Society of Clinical Neurophysiology 36, 21-27, doi:10.1007/s10072-014-1989-9 (2015).
26 Zuhlke, C., Riess, O., Bockel, B., Lange, H. & Thies, U. Mitotic stability and meiotic variability of the (CAG)n repeat in the Huntington disease gene. Human molecular genetics 2, 2063-2067 (1993).
27 Raymond, L. A. et al. Pathophysiology of Huntington's disease: time-dependent alterations in synaptic and receptor function. Neuroscience 198, 252-273, doi:10.1016/j.neuroscience.2011.08.052 (2011).
28 Andre, V. M. et al. Differential electrophysiological changes in striatal output neurons in Huntington's disease. J Neurosci 31, 1170-1182, doi:10.1523/JNEUROSCI.3539-10.2011 (2011).
29 Cepeda, C., Cummings, D. M., Andre, V. M., Holley, S. M. & Levine, M. S. Genetic mouse models of Huntington's disease: focus on electrophysiological mechanisms. ASN Neuro 2, e00033, doi:10.1042/AN20090058 (2010).
30 Silvestroni, A., Faull, R. L., Strand, A. D. & Moller, T. Distinct neuroinflammatory profile in post-mortem human Huntington's disease. Neuroreport 20, 1098-1103, doi:10.1097/WNR.0b013e32832e34ee (2009).
31 Xue, M. et al. Contributions of multiple proteases to neurotoxicity in a mouse model of intracerebral haemorrhage. Brain 132, 26-36, doi:10.1093/brain/awn215 (2009).
32 Li, S. H. et al. Lack of huntingtin-associated protein-1 causes neuronal death resembling hypothalamic degeneration in Huntington's disease. The Journal of neuroscience: the official journal of the Society for Neuroscience 23, 6956-6964 (2003).
33 Hodges, A. et al. Regional and cellular gene expression changes in human Huntington's disease brain. Hum Mol Genet 15, 965-977, doi:10.1093/hmg/dd1013 (2006).
34 Valenza, M. et al. Dysfunction of the cholesterol biosynthetic pathway in Huntington's disease. J Neurosci 25, 9932-9939, doi:10.1523/JNEUROSCI.3355-05.2005 (2005).
35 Lee, J. H. et al. Reinstating aberrant mTORC1 activity in Huntington's disease mice improves disease phenotypes. Neuron 85, 303-315, doi:10.1016/j.neuron.2014.12.019 (2015).
36 Tomas-Zapico, C. et al. alpha-Synuclein accumulates in huntingtin inclusions but forms independent filaments and its deficiency attenuates early phenotype in a mouse model of Huntington's disease. Hum Mol Genet 21, 495-510, doi:10.1093/hmg/ddr507 (2012).
37 Corrochano, S. et al. alpha-Synuclein levels modulate Huntington's disease in mice. Hum Mol Genet 21, 485-494, doi:10.1093/hmg/ddr477 (2012).
38 Strand, A. D. et al. Expression profiling of Huntington's disease models suggests that brain-derived neurotrophic factor depletion plays a major role in striatal degeneration. J Neurosci 27, 11758-11768, doi:10.1523/JNEUROSCI.2461-07.2007 (2007).
39 Zuccato, C. & Cattaneo, E. Role of brain-derived neurotrophic factor in Huntington's disease. Prog Neurobiol 81, 294-330, doi:10.1016/j.pneurobio.2007.01.003 (2007).
40 Plotkin, J. L. et al. Impaired TrkB receptor signaling underlies corticostriatal dysfunction in Huntington's disease. Neuron 83, 178-188, doi:10.1016j.neuron.2014.05.032 (2014).
41 Zhang, Q. et al. The Zinc Finger Transcription Factor Sp9 Is Required for the Development of Striatopallidal Projection Neurons. Cell Rep 16, 1431-1444, doi:10.1016/j.celrep.2016.06.090 (2016).
42 Bhide, P. G. et al. Expression of normal and mutant huntingtin in the developing brain. J Neurosci 16, 5523-5535 (1996).
43 Gutekunst, C. A. et al. Nuclear and neuropil aggregates in Huntington's disease: relationship to neuropathology. J Neurosci 19, 2522-2534 (1999).
44 Ko, J., Ou, S. & Patterson, P. H. New anti-huntingtin monoclonal antibodies: implications for huntingtin conformation and its binding proteins. Brain Res Bull 56, 319-329 (2001).
45 Legleiter, J. et al. Monoclonal antibodies recognize distinct conformational epitopes formed by polyglutamine in a mutant huntingtin fragment. J Biol Chem 284, 21647-21658, doi:10.1074jbc.M109.016923 (2009).
46 Macdonald, D. et al. Quantification assays for total and polyglutamine-expanded huntingtin proteins. PLoS One 9, e96854, doi:10.1371/joumal.pone.0096854 (2014).
47 Martinez-Vicente, M. et al. Cargo recognition failure is responsible for inefficient autophagy in Huntington's disease. Nat Neurosci 13, 567-576, doi:10.1038/nn.2528 (2010).
48 Zheng, S. et al. Deletion of the huntingtin polyglutamine stretch enhances neuronal autophagy and longevity in mice. PLoS genetics 6, e1000838, doi:10.1371journal.pgen.1000838 (2010).
49 Son, E. Y. et al. Conversion of mouse and human fibroblasts into functional spinal motor neurons. Cell Stem Cell 9, 205-218, doi:10.1016/j.stem.2011.07.014 (2011).
50 Taipale, M., Jarosz, D. F. & Lindquist, S. HSP90 at the hub of protein homeostasis: emerging mechanistic insights. Nat Rev Mol Cell Biol 11, 515-528, doi:10.1038/nrm2918 (2010).
51 Fujimoto, M. et al. Active HSF1 significantly suppresses polyglutamine aggregate formation in cellular and mouse models. J Biol Chem 280, 34908-34916, doi:10.1074jbc.M506288200 (2005).
52 Hayashida, N. et al. Heat shock factor 1 ameliorates proteotoxicity in cooperation with the transcription factor NFAT. EMBO J 29, 3459-3469, doi:10.1038/emboj.2010.225 (2010).
53 Gomez-Pastor, R. et al. Abnormal degradation of the neuronal stress-protective transcription factor HSF1 in Huntington's disease. Nat Commun 8, 14405, doi:10.1038/ncomms14405 (2017).
54 Vilchez, D. et al. Increased proteasome activity in human embryonic stem cells is regulated by PSMD11. Nature 489, 304-308, doi:10.1038/nature11468 (2012).
55 Olive, P. L. & Banath, J. P. The comet assay: a method to measure DNA damage in individual cells. Nature protocols 1, 23-29, doi:10.1038/nprot.2006.5 (2006).
56 Shiloh, Y. & Ziv, Y. The ATM protein kinase: regulating the cellular response to genotoxic stress, and more. Nature reviews. Molecular cell biology 14, 197-210, doi:10.1038/nrm3546 (2013).
57 Lu, X. H. et al. Targeting ATM ameliorates mutant Huntingtin toxicity in cell and animal models of Huntington's disease. Sci Transl Med 6, 268ra178, doi:10.1126/scitransimed.3010523 (2014).
58 Sesso, A. et al. Mitochondrial swelling and incipient outer membrane rupture in preapoptotic and apoptotic cells. Anat Rec (Hoboken) 295, 1647-1659, doi:10.1002/ar.22553 (2012).
59 Kim, I., Rodriguez-Enriquez, S. & Lemasters, J. J. Selective degradation of mitochondria by mitophagy. Arch Biochem Biophys 462, 245-253, doi:10.1016j.abb.2007.03.034 (2007).
60 Liu, L. et al. Glial lipid droplets and ROS induced by mitochondrial defects promote neurodegeneration. Cell 160, 177-190, doi:10.1016/j.cell.2014.12.019 (2015).
61 Gray, D. A. & Woulfe, J. Lipofuscin and aging: a matter of toxic waste. Science of aging knowledge environment: SAGE KE 2005, re1, doi:10.1126/sageke.2005.5.re1 (2005).
62 Goebel, H. H., Heipertz, R., Scholz, W., Iqbal, K. & Tellez-Nagel, I. Juvenile Huntington chorea: clinical, ultrastructural, and biochemical studies. Neurology 28, 23-31 (1978).
63 Kumar, A. & Ratan, R. R. Oxidative Stress and Huntington's Disease: The Good, The Bad, and The Ugly. J Huntingtons Dis 5, 217-237, doi:10.3233/JHD-160205 (2016).
64 Kegel, K. B. et al. Huntingtin expression stimulates endosomal-lysosomal activity, endosome tubulation, and autophagy. J Neurosci 20, 7268-7278(2000).
65 Vonsattel, J. P. et al. Neuropathological classification of Huntington's disease. Journal of neuropathology and experimental neurology 44, 559-577 (1985).
66 Kondo, T. et al. Modeling Alzheimer's disease with iPSCs reveals stress phenotypes associated with intracellular Abeta and differential drug responsiveness. Cell stem cell 12, 487-496, doi:10.1016j.stem.2013.01.009 (2013).
67 Ryan, S. D. et al. Isogenic human iPSC Parkinson's model shows nitrosative stress-induced dysfunction in MEF2-PGC1alpha transcription. Cell 155,1351-1364, doi:10.1016j.cell.2013.11.009 (2013).
68 Graybiel, A. M., Aosaki, T., Flaherty, A. W. & Kimura, M. The basal ganglia and adaptive motor control. Science 265, 1826-1831 (1994).
69 Reiner, A. et al. Differential loss of striatal projection neurons in Huntington disease. Proceedings of the National Academy of Sciences of the United States of America 85, 5733-5737 (1988).
70 Sathasivam, K. et al. Centrosome disorganization in fibroblast cultures derived from R62 Huntington's disease (HD) transgenic mice and HD patients. Human molecular genetics 10, 2425-2435 (2001).
71 Browne, S. E. & Beal, M. F. Oxidative damage in Huntington's disease pathogenesis. Antioxidants & redox signaling 8, 2061-2073, doi:10.1089/ars.2006.8.2061 (2006).
72 Dragunow, M. et al. In situ evidence for DNA fragmentation in Huntington's disease striatum and Alzheimer's disease temporal lobes. Neuroreport 6, 1053-1057 (1995).
73 GeM-HD. Identification of Genetic Factors that Modify Clinical Onset of Huntginton's Disease. Cell 162, 516-526, doi:10.1016/j.cell.2014.07.003 (2015).
74 Richner, M., Victor, M. B., Liu, Y., Abernathy, D. & Yoo, A. S. MicroRNA-based conversion of human fibroblasts into striatal medium spiny neurons. Nat Protoc 10, 1543-1555, doi:10.1038/nprot.2015.102 (2015).
75 Lobo, M. K., Karsten, S. L., Gray, M., Geschwind, D. H. & Yang, X. W. FACS-array profiling of striatal projection neuron subtypes in juvenile and adult mouse brains. Nat Neurosci 9, 443-452, doi:10.1038/nn1654 (2006).
76 Heiman, M. et al. A translational profiling approach for the molecular characterization of CNS cell types. Cell 135, 738-748, doi:10.1016/j.cell.2008.10.028 (2008).

What is claimed is:

1. A method of generating a neuron from an adult somatic cell comprising:
(i) providing an adult somatic cell, at least one miRNA capable of providing access to motor neuron genes in the adult somatic cell, and transcription factors;
(ii) providing the at least one miRNA to the adult somatic cell; and
(iii) providing the transcription factors to the adult somatic cell, resulting in conversion of the adult somatic cell into a converted neuron, wherein, the transcription factors are selected from the group consisting of:
motor neuron transcription factors ISL1 and LHX3; and
striatal-enriched factors CTIP2, DLX1, DLX2, and MYT1L (CDM).

2. The method of claim 1, wherein the adult somatic cell is an adult human fibroblast of mesodermal origin.

3. The method of claim 1, wherein the miRNA is selected from miR-9/9* and miR-124 (miR-9/9*-124).

4-5. (canceled)

6. The method of claim 1, wherein
the converted neuron is a motor neuron or a medium spiny neuron (MSN).

7. The method of claim 1, wherein the miRNA or the transcription factors are expressed in the adult somatic cell comprising an adult somatic cell genome by viral vector transduction.

8. The method of claim 7, wherein a viral vector expresses miRNA and an anti-apoptotic gene, beneficial for neuronal conversion, under an inducible promoter.

9. The method of claim 7, wherein
the miRNA or the transcription factors are cloned into a lentiviral plasmid;
a lentivirus comprising a lentivirus genome is produced and the adult somatic cell is infected;
the lentivirus genome comprises the miRNA or the transcription factors and is transfected into the adult somatic cell genome, resulting in a transduced adult somatic cell; and
the miRNA or the transcription factors are stably expressed by the transduced adult somatic cell.

10. The method of claim 1, wherein the miRNA or the transcription factors are administered exogenously to the adult somatic cells.

11. The method of claim 1, wherein the miRNA
coordinates epigenetic and transcriptional changes resulting in neuronal cell fate conversion;
induces a generic neuronal state characterized by loss of fibroblast identity, presence of a pan-neuronal gene expression program, and absence of subtype specificity;
initiates subunit switching within BAF chromatin remodeling complexes while separately repressing neuronal cell-fate inhibitors REST, Co-REST, and SCP1; or
alters expression of genes involved in DNA methylation, histone modifications, chromatin remodeling, and chromatin compaction.

12. The method of claim 1, wherein the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron.

13. The method of claim 1, wherein
the converted neuron phenotypically resembles an endogenous motor neurons when compared using immunostaining analysis or gene expression profiling;
the converted neuron resembles the endogenous motor neurons when compared using electrophysiological tests or co-culture tests; or
the converted neuron retains donor age marks and positional information from the adult somatic cell.

14. A method of modeling a neurodegenerative disease comprising:
(i) providing a fibroblast from a subject with a neurodegenerative disease; and
(ii) providing miR-9/9* and miR-124 (miR-9/9*-124) and transcription factors to the fibroblast, wherein the transcription factors are selected from the group consisting of:
motor neuron transcription factors ISL1 and LHX3; and
striatal-enriched factors CTIP2, DLX1, DLX2, and MYT1L (CDM).

15. The method of claim 14, wherein the neurodegenerative disease, disorder, or condition is selected from one or more of the group consisting of:
(i) a motor neuron disease;
(ii) spinal cord injury (SCI);
(iii) Amyotrophic Lateral Sclerosis (ALS) or Spinal Muscular Atrophy (SMA); or
(iv) Huntington's Disease (HD) or Alzheimer's Disease (AD).

16-20. (canceled)

21. A method of screening a candidate drug for effectiveness in treating a neurodegenerative or motor neuron disease comprising:
(i) providing a cellular platform, the cellular platform comprising neurons generated from fibroblasts of a subject with a neurodegenerative or motor neuron disease according to the method of claim 1;
(ii) providing a candidate drug;
(iii) contacting the candidate drug and the cellular platform; and
(iv) assessing efficacy of the candidate drug.

22. The method of claim 21, wherein the cellular platform comprises cells obtained from a subject with a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD).

23. The method of claim 21, wherein the efficacy is evaluated by monitoring the neurons for reversal of electrical impairment, spontaneous cell death, or stress-induced death.

24-27. (canceled)

28. A method of treating a neurodegenerative disease, disorder, or condition in a subject, comprising:
(i) providing an adult fibroblast;
(iii) administering a cell-conversion agent comprising miR-9/9* and miR-124 (miR-9/9*-124) and transcription factors to the adult fibroblast resulting in a converted neuron;
(iii) administering the converted neuron to the subject,
wherein, the transcription factors are selected from the group consisting of:
motor neuron transcription factors ISL1 and LHX3 and
striatal-enriched factors CTIP2, DLX1, DLX2, and MYT1L (CDM).

29. The method of claim 28, wherein the cell conversion agent is an exogenous agent or expressed in the adult fibroblast cell.

30. The method of claim 28, wherein the subject has or is suspected of having a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD).

31. The method of claim 28, wherein the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron.
